SEQUENCE LISTING (88 sequences)

SEQ ID NO: 1 - DNA, 3176 bp, Arabidopsis Thaliana
  CDS (51)..(2981)
cttttaaagtatatctaaaaacgcagtcgttttaagactgtgtgtgagaaatggctctgtttagagatattgttcttcttgggtttctcttctgcttgagcttagtagctactgtgacttcagaggagggagcaacgttgctggagattaagaagtcattcaaagatgtgaacaatgttctttatgactggacaacttcaccttcttcggattattgtgtctggagaggtgtgtcttgtgaaaatgtcaccttcaatgttgttgctcttaatttgtcagatttgaatcttgatggagaaatctcacctgctattggagatctcaagagtctcttgtcaattgatctgcgaggtaatcgcttgtctggacaaatccctgatgagattggtgactgttcttctttgcaaaacttagacttatccttcaatgaattaagtggtgacataccgttttcgatttcgaagttgaagcaacttgagcagctgattctgaagaataaccaattgataggaccgatcccttcaacactttcacagattccaaacctgaaaattctggacttggcacagaataaactcagtggtgagataccaagacttatttactggaatgaagttcttcagtatcttgggttgcgaggaaacaacttagtcggtaacatttctccagatttgtgtcaactgactggtctttggtattttgacgtaagaaacaacagtttgactggtagtatacctgagacgataggaaattgcactgccttccaggttttggacttgtcctacaatcagctaactggtgagatcccttttgacatcggcttcctgcaagttgcaacattatcattgcaaggcaatcaactctctgggaagattccatcagtgattggtctcatgcaagcccttgcagtcttagatctaagtggcaacttgttgagtggatctattcctccgattctcggaaatcttactttcaccgagaaattgtatttgcacagtaacaagctgactggttcaattccacctgagcttggaaacatgtcaaaactccattacctggaactcaatgataatcatctcacgggtcatataccaccagagcttgggaagcttactgacttgtttgatctgaatgtggccaacaatgatctggaaggacctatacctgatcatctgagctcttgcacaaatctaaacagcttaaatgttcatgggaacaagtttagtggcactataccccgagcatttcaaaagctagaaagtatgacttaccttaatctgtccagcaacaatatcaaaggtccaatcccggttgagctatctcgtatcggtaacttagatacattggatctttccaacaacaagataaatggaatcattccttcttcccttggtgatttggagcatcttctcaagatgaacttgagtagaaatcatataactggtgtagttccaggcgactttggaaatctaagaagcatcatggaaatagatctttcaaataatgatatctctggcccaattccagaagagcttaaccaattacagaacataattttgctgagactggaaaataataacctgactggtaatgttggttcattagccaactgtctcagtctcactgtattgaatgtatctcataacaacctcgtaggtgatatccctaagaacaataacttctcaagattttcaccagacagcttcattggcaatcctggtctttgcggtagttggctaaactcaccgtgtcatgattctcgtcgaactgtacgagtgtcaatctctagagcagctattcttggaatagctattgggggacttgtgatccttctcatggtcttaatagcagcttgccgaccgcataatcctcctccttttcttgatggatcacttgacaaaccagtaacttattcgacaccgaagctcgtcatccttcatatgaacatggcactccacgtttacgaggatatcatgagaatgacagagaatctaagtgagaagtatatcattgggcacggagcatcaagcactgtatacaaatgtgttttgaagaattgtaaaccggttgcgattaagcggctttactctcacaacccacagtcaatgaaacagtttgaaacagaactcgagatgctaagtagcatcaagcacagaaatcttgtgagcctacaagcttattccctctctcacttggggagtcttctgttctatgactatttggaaaatggtagcctctgggatcttcttcatggccctacgaagaaaaagactcttgattgggacacacggcttaagatagcatatggtgcagcacaaggtttagcttatctacaccatgactgtagtccaaggatcattcacagagacgtgaagtcgtccaacattctcttggacaaagacttagaggctcgtttgacagattttggaatagcgaaaagcttgtgtgtgtcaaagtcacatacttcaacttacgtgatgggcacgataggttacatagaccccgagtatgctcgcacttcacggctcactgagaaatccgatgtctacagttatggaatagtccttcttgagctgttaacccgaaggaaagccgttgatgacgaatccaatctccaccatctgataatgtcaaagacggggaacaatgaagtgatggaaatggcagatccagacatcacatcgacgtgtaaagatctcggtgtggtgaagaaagttttccaactggcactcctatgcaccaaaagacagccgaatgatcgacccacaatgcaccaggtgactcgtgttctcggcagttttatgctatcggaacaaccacctgctgcgactgacacgtcagcgacgctggctggttcgtgctacgtcgatgagtatgcaaatctcaagactcctcattctgtcaattgctcttccatgagtgcttctgatgctcaactgtttcttcggtttggacaagttatttctcagaacagtgagtagtttttcgttaggaggagaatctttaaaacggtatcttttcgttgcgttaagctgttagaaaaattaatgtctcatgtaaagtattatgcactgccttattattattagacaagtgtgtggtgtgaatatgtcttcagactggcacttagacttccaaaaaaaaaaaaaaaaaaaaaaaaaaaaaaaaaaaaaaaa

SEQ ID NO: 2 - protein, 976 aa, Arabidopsis Thaliana
MALFRDIVLLGFLFCLSLVATVTSEEGATLLEIKKSFKDVNNVLYDWTTSPSSDYCVWRGVSCENVTFNVVALNLSDLNLDGEISPAIGDLKSLLSIDLRGNRLSGQIPDEIGDCSSLQNLDLSFNELSGDIPFSISKLKQLEQLILKNNQLIGPIPSTLSQIPNLKILDLAQNKLSGEIPRLIYWNEVLQYLGLRGNNLVGNISPDLCQLTGLWYFDVRNNSLTGSIPETIGNCTAFQVLDLSYNQLTGEIPFDIGFLQVATLSLQGNQLSGKIPSVIGLMQALAVLDLSGNLLSGSIPPILGNLTFTEKLYLHSNKLTGSIPPELGNMSKLHYLELNDNHLTGHIPPELGKLTDLFDLNVANNDLEGPIPDHLSSCTNLNSLNVHGNKFSGTIPRAFQKLESMTYLNLSSNNIKGPIPVELSRIGNLDTLDLSNNKINGIIPSSLGDLEHLLKMNLSRNHITGVVPGDFGNLRSIMEIDLSNNDISGPIPEELNQLQNIILLRLENNNLTGNVGSLANCLSLTVLNVSHNNLVGDIPKNNNFSRFSPDSFIGNPGLCGSWLNSPCHDSRRTVRVSISRAAILGIAIGGLVILLMVLIAACRPHNPPPFLDGSLDKPVTYSTPKLVILHMNMALHVYEDIMRMTENLSEKYIIGHGASSTVYKCVLKNCKPVAIKRLYSHNPQSMKQFETELEMLSSIKHRNLVSLQAYSLSHLGSLLFYDYLENGSLWDLLHGPTKKKTLDWDTRLKIAYGAAQGLAYLHHDCSPRIIHRDVKSSNILLDKDLEARLTDFGIAKSLCVSKSHTSTYVMGTIGYIDPEYARTSRLTEKSDVYSYGIVLLELLTRRKAVDDESNLHHLIMSKTGNNEVMEMADPDITSTCKDLGVVKKVFQLALLCTKRQPNDRPTMHQVTRVLGSFMLSEQPPAATDTSATLAGSCYVDEYANLKTPHSVNCSSMSASDAQLFLRFGQVISQNSE

SEQ ID NO: 3 - DNA, 4199 bp, Arabidopsis Thaliana
atggctctgtttagagatattgttcttcttgggtttctcttctgcttgagcttagtagctactgtgacttcagaggagggtcagttattatactgatgcatgcttcttcaagttcaagattttcgtctttttgttttatattagtgaaaaaaacttaaagatgagatttttatatgatttttgaagtttcatttggtgaaaatgagatctgggtacttgttattttctatttttgctttttgtaatggtttttttttacttggtgggtcttctatagaatcaaaagaagctttgaataaattagggtttgagttttattttgttttcttggaagttgaatttttaatcttctcaagaactgacaaatatttttttttgtttttgtgcgtgtgtgttaataaaatatccttaaaacaaaattaaaggagcaacgttgctggagattaagaagtcattcaaagatgtgaacaatgttctttatgactggacaacttcaccttcttcggattattgtgtctggagaggtgtgtcttgtgaaaatgtcaccttcaatgttgttgctctgtaagtttcttcattcctttagattactattacagtggtttttggtgttcttgtgggaaaaagttgtaatttgttttgtgtgtgttttctatgttttgtagtaatttgtcagatttgaatcttgatggagaaatctcacctgctattggagatctcaagagtctcttgtcaatgtaactgtttcaacattcactgtagcatgaaataaagtatcttactttaattctattccactctctgagttgtgacttttgtcttctgtttttttctaatgtagtgatctgcgaggtaatcgcttgtctggacaaatccctgatgagattggtgactgttcttctttgcaaaacttgtaagaacagtgattggtgttattctaccattaaacttttgttcatagaggttttatttgatgaagtgtgttcatgttgtttttaattcagagacttatccttcaatgaattaagtggtgacataccgttttcgatttcgaagttgaagcaacttgagcagctgtaagtagctagttattctgctactagtcttcatatgtcattgctaaaaatatactcaccatgtggaatatggatttttactttgtccaggattctgaagaataaccaattgataggaccgatcccttcaacactttcacagattccaaacctgaaaattctgtatgttccccatgattcttacatgtcttactacttttagctatataggtgatcatacatgtgtaatttcaattgcagggacttggcacagaataaactcagtggtgagataccaagacttatttactggaatgaagttcttcagtatctgtaagtgtcaatgttttttgaagtctgtcaatgtctcttcattacccggtgataattgttgtactatgatgagcagtgggttgcgaggaaacaacttagtcggtaacatttctccagatttgtgtcaactgactggtctttggtatttgtgagtcttcttgcacatctgaatagtatgatgagttcttttgtaaatatcaaatatctgactttgttttgatattgaatcagtgacgtaagaaacaacagtttgactggtagtatacctgagacgataggaaattgcactgccttccaggttttgtatgtgcctctttctctacttctaaacatcattactgtaatttgggttacttaagaaaatctacttaactggtttgcttattacgaactcagggacttgtcctacaatcagctaactggtgagatcccttttgacatcggcttcctgcaagttgcaacattgttagttctcacctctactaatcttttgctttaaattttggctagcctttgttttcttttaaagaagatcattttcttatcttagatcattgcaaggcaatcaactctctgggaagattccatcagtgattggtctcatgcaagcccttgcagtcttgtaagtacttttcttctaatcaatgaagctacttataacattttcatgaacttaggttatatgttttcttttacagagatctaagtggcaacttgttgagtggatctattcctccgattctcggaaatcttactttcaccgagaaattgtaattctttacctgtttgttttcagtttggagtcaaatgtcataccatgttaatgatagtgatttatctttttggctttatctctaggtatttgcacagtaacaagctgactggttcaattccacctgagcttggaaacatgtcaaaactccattacctgtatgaccaaccttctcttcacttctctttttgcatacagtcactactaagttgtgtttccttatcaactatttgtaaaatattcatagggaactcaatgataatcatctcacgggtcatataccaccagagcttgggaagcttactgacttgtttgatctgtaagtagttcttcctatgcttgacatgttttgatgttcttatgcttatatgaactatgtacatataggaatgtggccaacaatgatctggaaggacctatacctgatcatctgagctcttgcacaaatctaaacagcttgtatgtatctctttctctgaaaacttctcacttgaatgttcaagattggtgctttatatgattttgtgtctcattaatgtaatgtagaaatgttcatgggaacaagtttagtggcactataccccgagcatttcaaaagctagaaagtatgacttacctgtaagtatcgacgctgagaatttctctaatcttatataatatatagttccacagcgtttgttttttcgaatttcaagtcattaactactgagtttttggttgcctttgatttatcggttcaaccagtaatctgtccagcaacaatatcaaaggtccaatcccggttgagctatctcgtatcggtaacttagatacattgtaagtgtttcttgttttctgtgaagtatacatcattatatgtgccttgtctcacatttattaaatttaatgacatttgaagggatctttccaacaacaagataaatggaatcattccttcttcccttggtgatttggagcatcttctcaagatgtgagcatccataagacctccagttttattgtttatttctagcaaaagatgaaaatggtttgtgaactcttgcattcttgttataggaacttgagtagaaatcatataactggtgtagttccaggcgactttggaaatctaagaagcatcatggaaatgtaagaagttaacttctatctgcttggttagagtttttttcatttatctcaattactgttctgaatttgtgtgtttgtggttgcagagatctttcaaataatgatatctctggcccaattccagaagagcttaaccaattacagaacataattttgctgtaagcaatcttcctcttatcccttccaagctgttaagaaattgtttttgtagaatgaaactaaaactctgtatacacaataatgaggtcactatagtgtgatccaggaacatgtattgggttggtgatctatctaatgttgtgtttcttaaaattgcttgcaggagactggaaaataataacctgactggtaatgttggttcattagccaactgtctcagtctcactgtattgtaagtaggcacctttggttctgaaacattttttgtccctctttgtgcatcttttgctaagaatataaccctgcaatcttcactaactcttataggaatgtatctcataacaacctcgtaggtgatatccctaagaacaataacttctcaagattttcaccagacaggtatggtaatttagcaggttttggtattgtgcattttgttttgtttgctaatatctatgtttatgtttttggataaagcttcattggcaatcctggtctttgcggtagttggctaaactcaccgtgtcatgattctcgtcgaactgtacgaggtgattacattcttctaaaagcttccattcacaaaacctaagataattaaagctcatgtttctatccatgttttgtctgcagtgtcaatctctagagcagctattcttggaatagctattgggggacttgtgatccttctcatggtcttaatagcagcttgccgaccgcataatcctcctccttttcttgatggatcatag

SEQ ID NO: 4 - protein, 614 aa, Arabidopsis Thaliana
MALFRDIVLLGFLFCLSLVATVTSEEGATLLEIKKSFKDVNNVLYDWTTSPSSDYCVWRGVSCENVTFNVVALNLSDLNLDGEISPAIGDLKSLLSIDLRGNRLSGQIPDEIGDCSSLQNLDLSFNELSGDIPFSISKLKQLEQLILKNNQLIGPIPSTLSQIPNLKILDLAQNKLSGEIPRLIYWNEVLQYLGLRGNNLVGNISPDLCQLTGLWYFDVRNNSLTGSIPETIGNCTAFQVLDLSYNQLTGEIPFDIGFLQVATLSLQGNQLSGKIPSVIGLMQALAVLDLSGNLLSGSIPPILGNLTFTEKLYLHSNKLTGSIPPELGNMSKLHYLELNDNHLTGHIPPELGKLTDLFDLNVANNDLEGPIPDHLSSCTNLNSLNVHGNKFSGTIPRAFQKLESMTYLNLSSNNIKGPIPVELSRIGNLDTLDLSNNKINGIIPSSLGDLEHLLKMNLSRNHITGVVPGDFGNLRSIMEIDLSNNDISGPIPEELNQLQNIILLRLENNNLTGNVGSLANCLSLTVLNVSHNNLVGDIPKNNNFSRFSPDSFIGNPGLCGSWLNSPCHDSRRTVRVSISRAAILGIAIGGLVILLMVLIAACRPHNPPPFLDGS

SEQ ID NO: 5 - DNA, 3100 bp, Arabidopsis Thaliana
  CDS (200)..(3100)
catctctggctctgcatttcttgcagttcacgctaatcacctgttttggtacccttttctctctgcaacaatggcagaacaagcctgaagtttcgttcttttgtgttttggtttcctctgctttgttgttattgaatcaaaacttcaacgagcttcttggcttattaccccaaaaggaaagaagaataagaagaaaaaaatgaaggagaagatgcagcgaatggttttatctttagcaatggtgggttttatggtttttggtgttgcttcggctatgaacaacgaagggaaagctctgatggcgataaaaggctctttcagcaacttagtgaatatgcttttggattgggacgatgttcacaacagtgacttgtgttcttggcgaggtgttttctgcgacaacgttagctactccgttgtctctctgaatttgtccagtctgaatcttggaggggagatatctccagctattggagacctacggaatttgcaatcaatagacttgcaaggtaataaactagcaggtcaaattccagatgagattggaaactgtgcttctcttgtttatctggatttgtccgagaatctgttatatggagacatacctttctcaatctctaaactcaagcagcttgaaactctgaatctgaagaacaatcagctcacaggtcctgtaccagcaaccttaacccagattccaaaccttaagagacttgatcttgctggcaatcatctaacgggtgagatatcgagattgctttactggaatgaagttttgcagtatcttggattacgagggaatatgttgactggaacgttatcttctgatatgtgtcagctaaccggtttgtggtactttgatgtgagaggaaataatctaactggaaccatcccggagagcatcggaaattgcacaagctttcaaatcctggacatatcttataatcagataacaggagagattccttacaatatcggcttcctccaagttgctactctgtcacttcaaggaaacagattgacgggtagaattccagaagttattggtctaatgcaggctcttgctgttttggatttgagtgacaatgagcttgttggtcctatcccaccgatacttggcaatctctcatttaccggaaagttgtatctccatggcaatatgctcactggtccaatcccctctgagcttgggaatatgtcacgtctcagctatttgcagctaaacgacaataaactagtgggaactattccacctgagcttggaaagctggagcaattgtttgaactgaatcttgccaacagccgtttagtagggcccataccatccaacattagttcatgtgcagccttgaatcaattcaatgttcatgggaacctcttgagtggatctattccactggcgtttcgcaatctcgggagcttgacttatctgaatctttcgtcgaacaatttcaagggaaaaataccagttgagcttggacatataatcaatcttgacaaactagatctgtctggcaataacttctcagggtctataccattaacgcttggcgatcttgaacaccttctcatattaaatcttagcagaaaccatcttagtggacaattacctgcagagtttgggaaccttcgaagcattcagatgattgatgtatcattcaatctgctctccggagttattccaactgaacttggccaattgcagaatttaaactctttaatattgaacaacaacaagcttcatgggaaaattccagatcagcttacgaactgcttcactcttgtcaatctgaatgtctccttcaacaatctctccgggatagtcccaccaatgaaaaacttctcacgttttgctccagccagctttgttggaaatccatatctttgtggaaactgggttggatctatttgtggtcctttaccgaaatctcgagtattctccagaggtgctttgatctgcattgttcttggcgtcatcactctcctatgtatgattttccttgcagtttacaaatcaatgcagcagaagaagattctacaaggctcctcaaaacaagctgaagggttaaccaagctagtgattctccacatggacatggcaattcatacatttgatgatatcatgagagtgactgagaatcttaacgaaaagtttataattggatatggtgcttctagcacggtatacaaatgtgcattaaaaagttcccgacctattgccattaagcgactctacaatcagtatccgcataacttgcgggaatttgagacagaacttgagaccattgggagcattaggcacagaaacatagtcagcttgcatggatatgccttgtctcctactggcaaccttcttttctatgactacatggaaaatggatcactttgggaccttcttcatgggtcattgaagaaagtgaagcttggttgggagacaaggttgaagatagcggttggagctgcacaaggactagcctatcttcaccacgattgtactcctcgaatcattcaccgtgacatcaagtcatcgaacatacttcttgatgagaatttcgaagcacacttatctgatttcgggattgctaagagcataccagctagcaaaacccatgcctcgacttatgttttgggaacaattggttatatagacccagagtatgctcgtacttcacgaatcaatgagaaatccgatatatacagcttcggtattgttcttcttgagcttctcactgggaagaaagcagtggataacgaagctaacttgcatcaactgatattgtcaaaggctgatgataatactgtgatggaagcagttgatccagaggttactgtgacttgtatggacttgggacatatcaggaagacatttcagctggctctcttatgcacaaagcgaaaccctttagagagacccacaatgcttgaagtctctagggttctgctctctcttgtcccatctctgcaagtagcaaagaagctaccttctcttgatcactcaaccaaaaagctgcagcaagagaatgaagttaggaatcctgatgcagaagcatctcaatggtttgttcagttccgtgaagtcatctccaaaagtagcatataa

SEQ ID NO: 6 - protein, 966 aa, Arabidopsis Thaliana
MKEKMQRMVLSLAMVGFMVFGVASAMNNEGKALMAIKGSFSNLVNMLLDWDDVHNSDLCSWRGVFCDNVSYSVVSLNLSSLNLGGEISPAIGDLRNLQSIDLQGNKLAGQIPDEIGNCASLVYLDLSENLLYGDIPFSISKLKQLETLNLKNNQLTGPVPATLTQIPNLKRLDLAGNHLTGEISRLLYWNEVLQYLGLRGNMLTGTLSSDMCQLTGLWYFDVRGNNLTGTIPESIGNCTSFQILDISYNQITGEIPYNIGFLQVATLSLQGNRLTGRIPEVIGLMQALAVLDLSDNELVGPIPPILGNLSFTGKLYLHGNMLTGPIPSELGNMSRLSYLQLNDNKLVGTIPPELGKLEQLFELNLANSRLVGPIPSNISSCAALNQFNVHGNLLSGSIPLAFRNLGSLTYLNLSSNNFKGKIPVELGHIINLDKLDLSGNNFSGSIPLTLGDLEHLLILNLSRNHLSGQLPAEFGNLRSIQMIDVSFNLLSGVIPTELGQLQNLNSLILNNNKLHGKIPDQLTNCFTLVNLNVSFNNLSGIVPPMKNFSRFAPASFVGNPYLCGNWVGSICGPLPKSRVFSRGALICIVLGVITLLCMIFLAVYKSMQQKKILQGSSKQAEGLTKLVILHMDMAIHTFDDIMRVTENLNEKFIIGYGASSTVYKCALKSSRPIAIKRLYNQYPHNLREFETELETIGSIRHRNIVSLHGYALSPTGNLLFYDYMENGSLWDLLHGSLKKVKLGWETRLKIAVGAAQGLAYLHHDCTPRIIHRDIKSSNILLDENFEAHLSDFGIAKSIPASKTHASTYVLGTIGYIDPEYARTSRINEKSDIYSFGIVLLELLTGKKAVDNEANLHQLILSKADDNTVMEAVDPEVTVTCMDLGHIRKTFQLALLCTKRNPLERPTMLEVSRVLLSLVPSLQVAKKLPSLDHSTKKLQQENEVRNPDAEASQWFVQFREVISKSSI

SEQ ID NO: 7 - DNA, 3089 bp, Arabidopsis Thaliana
  CDS (186)..(3089)
tctcccaacaatggcagaacgactttgtacccttcttttgctctttgtttgaatttcgtttcttgctacaaagcttcaaaggatctgacttttccctaaacagaaaaagaggtctttaaccaaaaaaggttgttacttgttttctgggtttcgtggtgttactcttgaggaagaagaagaagaagatgagaaggatagagaccatgaaaggcttgtttttttgtctggggatggtggttttcatgctacttggttctgtttctccaatgaacaacgaaggaaaagcgttgatggcgataaaggcttcattcagcaacgtggcgaatatgcttcttgattgggacgatgttcataaccacgacttttgttcttggagaggtgtcttctgtgataacgttagcctcaatgttgtctctcttaatctgtcaaacctgaatcttggtggagagatatcatctgcccttggagatttgatgaatctgcaatcaatagacttgcaaggaaataaattgggtggtcaaattccagatgagattggaaactgtgtttctcttgcttatgtggatttctccaccaatttgttgtttggagacataccgttttcaatctctaaactcaaacagctggagtttctgaacctaaagaataatcagctcactggtccaataccagcaaccttaactcagattccaaaccttaagacccttgacctcgcaagaaaccagcttactggtgagataccaaggttactctactggaatgaagttttacagtatctcggtttacgtgggaatatgttaactgggacattgtctcctgatatgtgtcagctgacgggtctgtggtactttgatgtgagaggcaacaaccttactggaactatcccagagagcattggcaattgcacaagctttgagatcttggatgtatcttataatcagattaccggagttataccctacaatattggtttcctccaagtagctactctgtcacttcaaggaaacaagttgactggcagaattccggaagtgattggtctgatgcaggctcttgctgtattggatttgagtgacaatgaattaactgggcctattccaccaatacttgggaatctgtcattcactggaaaactgtatctccatggcaacaagctcactggacaaatcccacccgagctaggcaatatgtcacgactcagctatttgcaactaaatgataatgaactagtgggaaagatcccacctgagcttgggaagctggaacaattgttcgaactgaatcttgcgaacaacaatcttgtagggctgattccatctaacattagttcctgtgctgccttgaatcaattcaatgttcatgggaacttcttgagtggagctgtaccacttgaattccggaatcttggaagcttgacttatctaaatctttcctcaaacagtttcaagggcaaaatacctgctgagcttggccatatcatcaatcttgatacattggatctgtctggcaacaatttctcaggctcaattccattaacacttggtgatcttgagcatcttctcatcttaaacttgagcagaaatcatctgaatggcacattgcctgcagaattcgggaacctccgaagcattcagatcatcgatgtgtcatttaattttcttgccggtgttattccaactgaacttggccagttgcagaacataaactctctgatactgaacaacaacaagattcatgggaaaatccctgatcagctaactaactgcttcagtcttgccaatctgaacatctccttcaataatctttctggaataatcccacctatgaagaactttacacgtttttccccggccagcttctttggaaatccatttctctgcgggaactgggttggatcaatctgtggcccatctttacctaagtcacaagtattcaccagagttgccgtgatttgtatggttctcggtttcatcactctcatatgcatgatattcattgcggtttacaagtcaaagcagcagaaaccagtcttgaaaggctcttcaaaacaacctgaagggtcaacgaagctggtgattcttcacatggacatggctattcacacgtttgatgatatcatgagagttacagaaaacctcgatgagaaatacatcattggatacggtgcttctagcacagtttacaagtgcacctccaaaacttcccgacctattgccattaagcgaatctacaatcagtatcccagcaacttccgcgagtttgaaacagagctcgagaccattgggagcatcagacacagaaacatagtaagcttgcacggatacgccttatctccctttggcaacctcctcttctacgactacatggaaaatggctctctttgggatcttctccatgggcctgggaagaaggtgaagcttgactgggaaacaaggctgaagatagctgttggagctgcgcaaggacttgcatatcttcaccatgactgcacacctaggataatccatcgagacatcaagtcatcaaacatactccttgatgggaatttcgaagcgcgtttgtcagattttgggattgccaagagcataccagccaccaaaacttatgcttcaacctatgttcttggaaccattggatatattgacccagagtatgctcgaacttcgcgtctgaacgagaagtctgatatctacagtttcggtattgtccttcttgagcttctaaccggcaagaaggctgtggataacgaggccaacttgcatcaaatgattctatcaaaggcggatgataacacagtaatggaagctgttgatgcagaggtctcagtgacttgcatggactcaggacacatcaagaaaacatttcagctagctctcttgtgcaccaagcgaaatcctttggagagacccaccatgcaggaggtctctagggttctgctctcacttgtcccgtctccacctccaaagaagttaccgtcgcctgcaaaagtacaggaaggggaagaacggcgtgagagccactcttcagatacaacaaccccacagtggtttgttcagttccgtgaagatatctccaaaagtagcttataa

SEQ ID NO: 8 - protein, 967 aa, Arabidopsis Thaliana
MRRIETMKGLFFCLGMVVFMLLGSVSPMNNEGKALMAIKASFSNVANMLLDWDDVHNHDFCSWRGVFCDNVSLNVVSLNLSNLNLGGEISSALGDLMNLQSIDLQGNKLGGQIPDEIGNCVSLAYVDFSTNLLFGDIPFSISKLKQLEFLNLKNNQLTGPIPATLTQIPNLKTLDLARNQLTGEIPRLLYWNEVLQYLGLRGNMLTGTLSPDMCQLTGLWYFDVRGNNLTGTIPESIGNCTSFEILDVSYNQITGVIPYNIGFLQVATLSLQGNKLTGRIPEVIGLMQALAVLDLSDNELTGPIPPILGNLSFTGKLYLHGNKLTGQIPPELGNMSRLSYLQLNDNELVGKIPPELGKLEQLFELNLANNNLVGLIPSNISSCAALNQFNVHGNFLSGAVPLEFRNLGSLTYLNLSSNSFKGKIPAELGHIINLDTLDLSGNNFSGSIPLTLGDLEHLLILNLSRNHLNGTLPAEFGNLRSIQIIDVSFNFLAGVIPTELGQLQNINSLILNNNKIHGKIPDQLTNCFSLANLNISFNNLSGIIPPMKNFTRFSPASFFGNPFLCGNWVGSICGPSLPKSQVFTRVAVICMVLGFITLICMIFIAVYKSKQQKPVLKGSSKQPEGSTKLVILHMDMAIHTFDDIMRVTENLDEKYIIGYGASSTVYKCTSKTSRPIAIKRIYNQYPSNFREFETELETIGSIRHRNIVSLHGYALSPFGNLLFYDYMENGSLWDLLHGPGKKVKLDWETRLKIAVGAAQGLAYLHHDCTPRIIHRDIKSSNILLDGNFEARLSDFGIAKSIPATKTYASTYVLGTIGYIDPEYARTSRLNEKSDIYSFGIVLLELLTGKKAVDNEANLHQMILSKADDNTVMEAVDAEVSVTCMDSGHIKKTFQLALLCTKRNPLERPTMQEVSRVLLSLVPSPPPKKLPSPAKVQEGEERRESHSSDTTTPQWFVQFREDISKSSL

SEQ ID NO: 9 - DNA, 4399 bp, Arabidopsis Thaliana
atgttggagaagatgcagcgaatggttttatctttagcaatggtgggttttatggtttttggtgttgcttcggctatgaacaacgaaggtttgtttcttctaactcttttgtttttgtcttttttcttgtgatctctgtttaaaattttgattatgtgcttcaaaaattagggtttagttaggtatatctccttaaacccattctctgattttttctgaaagtttgaatctaaaatcgtctgtgttagaagttatcatgacatataatctttggtttatgtcaaacatttggtttccccataaaaagagagttaaagttgaaagctttttcatacccaatacttagctctggcttgttcttcatcaaatgcatataaagatgcaatcttttctccatttcccaagaaatttgatcatgattagtacacagagaatgcctgttcctgtaccatggaaagtttgtgtttttttcaagctgtttcccgagaaaaaaacgaagcttaatccctagtgtcctggagagaaaggacatgaaatttacaaaaattccttagttttggtttatgtatatttaactatgtgactgatgttccgcgtttcaaatgatttattactaatcctagttgagtctgtttatgaattttgaaaacccacagggaaagctctgatggcgataaaaggctctttcagcaacttagtgaatatgcttttggattgggacgatgttcacaacagtgacttgtgttcttggcgaggtgttttctgcgacaacgttagctactccgttgtctctctgtaagaatcttatttgagttcaataataattcttgtgttgttgaaaaatgtctctcttttgtggtcttgtaatattctgctttttttctatttttactttgtgaaggaatttgtccagtctgaatcttggaggggagatatctccagctattggagacctacggaatttgcaatcaatgtaatggttttttttccctttctttctctgttgatttgcatgtttgaactttgaaatgttgtagcttttgttcataaaggctttttccaattgcagagacttgcaaggtaataaactagcaggtcaaattccagatgagattggaaactgtgcttctcttgtttatctgtaagtcttattaaacttgtttttttgagttttcaattttcatgtctttagttttgacgtccatatcctctgttgtacagggatttgtccgagaatctgttatatggagacatacctttctcaatctctaaactcaagcagcttgaaactctgtatgtgttatacttcaacattttgtttttccgattcaaagttcaaattttgtctacttgttctttttttttttcgagtggatttttgtgaacctttgaacttgtggatgtatcaggaatctgaagaacaatcagctcacaggtcctgtaccagcaaccttaacccagattccaaaccttaagagactgtgagttctttagttacatcatttactcatgaatcaaaatttgggaatagattttgaacttactcattttcttttttccaatcatgatgttgcagtgatcttgctggcaatcatctaacgggtgagatatcgagattgctttactggaatgaagttttgcagtatctgtaagttcgtcagagtcattttctctgatctcaatatagtctacaccatagaagaagttaattttctccttctaatactttctttctgcagtggattacgagggaatatgttgactggaacgttatcttctgatatgtgtcagctaaccggtttgtggtacttgtaagtggatccaacgctctgccatcttttgagtcttgtatagatcctggtccttctttgattcattcaagtcttgagttatattttttaacttgtgttttgttgttggaagtgatgtgagaggaaataatctaactggaaccatcccggagagcatcggaaattgcacaagctttcaaatcctgtatgtgccatcttaaattaaacattagacttccactataggagtgaattttggactcataccttttgatattacagggacatatcttataatcagataacaggagagattccttacaatatcggcttcctccaagttgctactctgtgagtattgagcctcttttaccttagtttcatcagtaaattgttaacatgcggttattgaattgtgtattattactttttgaaggtcacttcaaggaaacagattgacgggtagaattccagaagttattggtctaatgcaggctcttgctgttttgtaagttcaattcatatggaaaagtaacgacaaagtacattcttgaatttggctcgtctgactttgtgaatcctattcaattcacagggatttgagtgacaatgagcttgttggtcctatcccaccgatacttggcaatctctcatttaccggaaagttgtgagtcttttttgtataactgtagtatctcatatgttcagctaatcagctcttggcatatgcattgataccttttttttttgttgaaataggtatctccatggcaatatgctcactggtccaatcccctctgagcttgggaatatgtcacgtctcagctatttgtaagcttgttttaaacttcaaatctgtttacatagctttcgcattccatatacttatctattttgcttttcttttaccgattttgaaggcagctaaacgacaataaactagtgggaactattccacctgagcttggaaagctggagcaattgtttgaactgtgagtttcttttccttgctctaaagtttttcactttagctgttgaagcattacatttgatccttgtttttactgatccattttttgacatgaaaggaatcttgccaacaaccgtttagtagggcccataccatccaacattagttcatgtgcagccttgaatcaattgtaagctataagatagttcattttgaattcatgctttactcttcttattcacaaacctaaattctgctgctctacttgaattcagcaatgttcatgggaacctcttgagtggatctattccactggcgtttcgcaatctcgggagcttgacttatctgtgagtttcattcaatacaatgctgtaacattgcttccctctaaacattatagtgactgttgaatccttttggatgcaggaatctttcgtcgaacaatttcaagggaaaaataccagttgagcttggacatataatcaatcttgacaaactgtatgtcttctcaactttgttacatcgtttggttttatgtttcagtttgaatttgctgcaaactcatcatatgtttttgtttccagagatctgtctggcaataacttctcagggtctataccattaacgcttggcgatcttgaacaccttctcatattgtaagtcttttacacttaaagttgtaaatttgtaaatgtttttcttgtgtttgttaacttttaatcttttgcttcattaaacagaaatcttagcagaaaccatcttagtggacaattacctgcagagtttgggaaccttcgaagcattcagatgatgtaaagcttatagttgcttcttttggttatatcaaaatcatattttttttctgacttatctgttttacttgtagtgatgtatcattcaatctgctctccggagttattccaactgaacttggccaattgcagaatttaaactctttgtaagttaataatatgatcttatcatttgtattatatacaaaatagatgtatggtttgatctttatgtttatgtacagaatattgaacaacaacaagcttcatgggaaaattccagatcagcttacgaactgcttcactcttgtcaatctgtaagttcttctgcaagagatacttgcttatgagcaatattagccctttggaagatttgtgaatccaacattttcttcttctttcttcgaaaacgttttaggaatgtctccttcaacaatctctccgggatagtcccaccaatgaaaaacttctcacgttttgctccagccaggtatgaagcttctctcagtagtctgtcattatgatcaatacgcataagctttcattgtaaaactaaattcataacctctgtatagctttgttggaaatccatatctttgtggaaactgggttggatctatttgtggtcctttaccgaaatctcgaggtatctactagatttatccattaagcattttcttcaatagatgttttttcttgtttatttgagcttgcttttcttgttcttttcagtattctccagaggtgctttgatctgcattgttcttggcgtcatcactctcctatgtatgattttccttgcagtttacaaatcaatgcagcagaagaagattctacaaggctcctag

SEQ ID NO: 10 - protein, 616 aa, Arabidopsis Thaliana
MKEKMQRMVLSLAMVGFMVFGVASAMNNEGKALMAIKGSFSNLVNMLLDWDDVHNSDLCSWRGVFCDNVSYSVVSLNLSSLNLGGEISPAIGDLRNLQSIDLQGNKLAGQIPDEIGNCASLVYLDLSENLLYGDIPFSISKLKQLETLNLKNNQLTGPVPATLTQIPNLKRLDLAGNHLTGEISRLLYWNEVLQYLGLRGNMLTGTLSSDMCQLTGLWYFDVRGNNLTGTIPESIGNCTSFQILDISYNQITGEIPYNIGFLQVATLSLQGNRLTGRIPEVIGLMQALAVLDLSDNELVGPIPPILGNLSFTGKLYLHGNMLTGPIPSELGNMSRLSYLQLNDNKLVGTIPPELGKLEQLFELNLANSRLVGPIPSNISSCAALNQFNVHGNLLSGSIPLAFRNLGSLTYLNLSSNNFKGKIPVELGHIINLDKLDLSGNNFSGSIPLTLGDLEHLLILNLSRNHLSGQLPAEFGNLRSIQMIDVSFNLLSGVIPTELGQLQNLNSLILNNNKLHGKIPDQLTNCFTLVNLNVSFNNLSGIVPPMKNFSRFAPASFVGNPYLCGNWVGSICGPLPKSRVFSRGALICIVLGVITLLCMIFLAVYKSMQQKKILQGS

SEQ ID NO: 11 - DNA, 4147 bp, Arabidopsis Thaliana
atgttaaggatagagaccatgaaaggcttgtttttttgtctggggatggtggttttcatgctacttggttctgtttctccaatgaacaacgaaggttctagctctttcaaaatctcttttgtttctctattgttccatgagccagagagatttctctcttttttttccagagaacttgaatttttataaatttgtaacctttcttccgattttgaagatatcatatctgtttatctgtctatctaaacagtgactgttcctgagagagcaaaagtcatttgattcgttcttaaaaaaaacttactttagtgtttttggaaatatgataataatttgtgacctagctcacttcacagttgagtttattatactaatgatctagtggggttttagtttttggcatgtggttcatattttaccatttgactatcatcagtgtcaaatgattaaataagctttttttgatttgtttattgacttcaaaaagcttttgattttgcacaggaaaagcgttgatggcgataaaggcttcattcagcaacgtggcgaatatgcttcttgattgggacgatgttcataaccacgacttttgttcttggagaggtgtcttctgtgataacgttagcctcaatgttgtctctctgtaaggattttttttttatctcattccgtattgttgtgctgtctctttgatttcagacttttctgatttgcctttgtttttttttatgttggaagtaatctgtcaaacctgaatcttggtggagagatatcatctgcccttggagatttgatgaatctgcaatcaatgttcgtttctcttctctcttttacttgtatgtttgagaagaaacatggtgttaaatccttatgaagctctggtctttcttaattacagagacttgcaaggaaataaattgggtggtcaaattccagatgagattggaaactgtgtttctcttgcttatgtgtaagttttgtttgatgcttgtagtttcatgttaatgagaacttaatctatctctattatataatcaaaaacctttcttgtttcagggatttctccaccaatttgttgtttggagacataccgttttcaatctctaaactcaaacagctggagtttctgtatgtcttcttatctctctcattcgtgtgtaatctttgcctgattttggcgtttttttgtcaactgtttcattctaatgcaggaacctaaagaataatcagctcactggtccaataccagcaaccttaactcagattccaaaccttaagaccctgtgagtttcaatcagataatattctatctatgaagcaggttatttaatgcaaagggatcattgcaatgataatagattattctcatgattttgcagtgacctcgcaagaaaccagcttactggtgagataccaaggttactctactggaatgaagttttacagtatctgtaagtagttcatctacttatcttaagtctaggcatccagagatttatgttctgattgtgatgtattcattttgcagcggtttacgtgggaatatgttaactgggacattgtctcctgatatgtgtcagctgacgggtctgtggtacttgtaagtgaatccggttcagctctttattttcttttactctcaacctcagatttcgagtaatttataagtgaatgtttgttgttgttagtgatgtgagaggcaacaaccttactggaactatcccagagagcattggcaattgcacaagctttgagatcttgtatgtgtcttctccttaaggaatgaatattcagcacagaactcgagtgatcttattcgtcatttgcattttcagggatgtatcttataatcagattaccggagttataccctacaatattggtttcctccaagtagctactctgtaagtattgttaagaccatcttacacttttgtgtggtttgttaaagcatgtcaatatggttaagtggtgatgggttctcttgcaggtcacttcaaggaaacaagttgactggcagaattccggaagtgattggtctgatgcaggctcttgctgtattgtatgtcattttcatattcaaatgcttgcttttttcctactctcgtttcttgctcttctgactgaatgaaataaacttatccaatgcgcagggatttgagtgacaatgaattaactgggcctattccaccaatacttgggaatctgtcattcactggaaaactgtgagtccctttttttttcttccctctgtgtcatgcatatgttcagatcttcacatatatggataatgtcttttcttgaaaataggtatctccatggcaacaagctcactggacaaatcccacccgagctaggcaatatgtcacgactcagctatttgtaagattgagaacttgaaactccaatatctgttcttctcaatacttgatcttatgagggtttactctttcgcaggcaactaaatgataatgaactagtgggaaagatcccacctgagcttgggaagctggaacaattgttcgaactgtgagtttttatccttcagtatacttaaatttttatcttaggtgaaaagctggttgatgtaactttgccatctaggaatcttgcgaacaacaatcttgtagggctgattccatctaacattagttcctgtgctgccttgaatcaattgtaagatatgatatgaaccctttcccattttatgcttctctctatttttcttcttcttatcttaatgaaaacttattgtgcttttattgtgttcagcaatgttcatgggaacttcttgagtggagctgtaccacttgaattccggaatcttggaagcttgacttatctgtaagcttcaatgactgcatgtacagtatgattatgtattcgaactattagtctgtgtaaatcttgattcaatgctgtaaattgcagaaatctttcctcaaacagtttcaagggcaaaatacctgctgagcttggccatatcatcaatcttgatacattgtatgttattttctactgatttacatccatgttccgcctagttacctactgatatgtgtaccaactaccaactcatacaaactttgtttcagggatctgtctggcaacaatttctcaggctcaattccattaacacttggtgatcttgagcatcttctcatcttgtatgtcttcatcaagccctaaagttgcactgctttgcattaactctctcagatttgttgacttgagttttgttttaattgtcagaaacttgagcagaaatcatctgaatggcacattgcctgcagaattcgggaacctccgaagcattcagatcatgtaaggcttgtcttatgcttagatttcttctgttatgataaaatcttcctacttacaaacttttctccatccttctcagcgatgtgtcatttaattttcttgccggtgttattccaactgaacttggccagttgcagaacataaactctctgtaagtaaaccatcacttctcctctgcaatttaccgtataatgcatataaataattcttctttgcttctttaatgtacaggatactgaacaacaacaagattcatgggaaaatccctgatcagctaactaactgcttcagtcttgccaatctgtacgtgttcctttgttacaacaagatgtgtactctcagataatcttatgatcatttctgaattcatatatttttcctgctcgttctttatacattgtaggaacatctccttcaataatctttctggaataatcccacctatgaagaactttacacgtttttccccggccaggtatcacctttgccatattgttacctgtcttagcttttcatataatcagtataccaaatataccttttcgtttatagcttctttggaaatccatttctctgcgggaactgggttggatcaatctgtggcccatctttacctaagtcacaaggtatatgaatctgcatttatcctattgcattgtttttagctgacatatctctcactacatttcttgctcttgaccaaccagtattcaccagagttgccgtgatttgtatggttctcggtttcatcactctcatatgcatgatattcattgcggtttacaagtcaaagcagcagaaaccagtcttgaaaggctcttag

SEQ ID NO: 12 - protein, 619 aa, Arabidopsis Thaliana
MRRIETMKGLFFCLGMVVFMLLGSVSPMNNEGKALMAIKASFSNVANMLLDWDDVHNHDFCSWRGVFCDNVSLNVVSLNLSNLNLGGEISSALGDLMNLQSIDLQGNKLGGQIPDEIGNCVSLAYVDFSTNLLFGDIPFSISKLKQLEFLNLKNNQLTGPIPATLTQIPNLKTLDLARNQLTGEIPRLLYWNEVLQYLGLRGNMLTGTLSPDMCQLTGLWYFDVRGNNLTGTIPESIGNCTSFEILDVSYNQITGVIPYNIGFLQVATLSLQGNKLTGRIPEVIGLMQALAVLDLSDNELTGPIPPILGNLSFTGKLYLHGNKLTGQIPPELGNMSRLSYLQLNDNELVGKIPPELGKLEQLFELNLANNNLVGLIPSNISSCAALNQFNVHGNFLSGAVPLEFRNLGSLTYLNLSSNSFKGKIPAELGHIINLDTLDLSGNNFSGSIPLTLGDLEHLLILNLSRNHLNGTLPAEFGNLRSIQIIDVSFNFLAGVIPTELGQLQNINSLILNNNKIHGKIPDQLTNCFSLANLNISFNNLSGIIPPMKNFTRFSPASFFGNPFLCGNWVGSICGPSLPKSQVFTRVAVICMVLGFITLICMIFIAVYKSKQQKPVLKGS

SEQ ID NO: 13 - DNA, 1802 bp, Arabidopsis Thaliana
gaattcaaaggaataagcatcggagacgatttaatgttacctcttgacgtatttatccaatttatccattaagccaccagccatagcatctgatcatcatcatcaacatataaataaccaaatttgaaatgaacaaaagtcgaattggtgatattgaaaatcgagttcgtgaaattgagaatcggattggtgaatttgaagagagatgcgtgtaccgttagggaggaggaggagacgggagagaaaaaaggagacggagataactcgccggctctgtttccatggcggaggtgataatgtagctgcgcacgttagctttttgtggtttgagttggagaacagtgggaggctcacggtagcgtggagtgacgacattggggataacaccagaggcgtcttatctccgttggacaaattattattatggctatgaacattcaacatataatttaattagaaatttgcggatgaaaaagaggtaaacaattgcagaaatggttaaaaatattaacgttgtacagcaaatgataataaaaagtgtaacgtacagtgtgtaaggaatggaaaaataataatttgggttaaaataaatatgtagttttctaactatatagtactttttgagaaaagataatattatgtgtatttttattgaaacaaataaatgatttaacaaaaaaaaaaagagaagttaaaatgaaaaggaattattattttttaagttcttccttcttttgttgggcctgtgacccttttagttttagtccacttcgttctcaaagcttcaaaatattaattttgtgacaaaccgaccggagccaaccaaaccggttaacatcctaaaaccaatcatattttattaagttttgtgttgatgctaaaccaaaaatcattggcatgcatatttctaaatttagtaataaacaaaaacacttagaaatcacacgttcactatactaaaaaacgttgacaaaaacacaacaactatactaataattaaagaagagaaaactgaaccaaactttttgtaaactcctgaatttaaattagtaattgaagtaagaagatgaagaagaacatgttaagcaaacaaaaaaattacactaaaatcatataaaaatacataattacaaaagtacccataagatggatttattgatatgggtcatctgtgaaacaagccacagagagacaaagactcgtaagtattgggcaacgaaagcgacctcctttattcaccactgccattaacatgttcttcttctccttcttcttctacattttatgaccgttttacccttcaagagagagaaacaaaatcactccctctcactcactctatctctctcttctgcaaagcttcagaactctggcagagagataaaagatgatggggtttttaactttatcctccccaaataattcttcttcccttcatctctctctcttacacaacaggtccctacatttgtacaatctcctctctttaaagactctctctctttctctctccatctctatcttactctgtatttctgtcgtctgagcactcaatgaaaccactgtaaatttccgccagaatttgatgtgatggaacgataaaaatcattttttctcggttaaagtaaaaaaacaaaaacaaatttctgtagaaatcataataaaagaaagaaaaaaaatctaatgtcggtacataatacggttctcttcttcttctctatcctctgtttcttcttcatggagacttgaaagcttttaaagtatatctaaaaacgcagtcgttttaagactgtgtgtgagaa

SEQ ID NO: 14 - DNA, 1963 bp, Arabidopsis Thaliana
ttttcgttaggaggagaatctttaaaacggtatcttttcgttgcgttaagctgttagaaaaattaatgtctcatgtaaagtattatgcactgccttattattattagacaagtgtgtggtgtgaatatgtcttcagactggcacttagacttcctataagttcttgcctatctaagtttttctaaattgggttattcttgtaacatatcttagatctagtactcaacaccacgtcaccaccacaaaagatttcttatgctcaaaaacatatacatagaaagaaccttctaaactacgagaaacgttttgctatgtagtgttatatgtcaaccacgtctatgagagtgcaaacgataggttaataagttttctcacttggcaataaaaatgataaacaaatatattgtctgattaatttattttatatagtttttttataatttcttatattaattcgaactcatacagcgcgtgagactttctagtttagtataaagtacgtatttttgcaaaatcaaaatcgtaaatacatacattttaaaatgttaaaaaagataaatccgtacaccatttaaaaatggcattttcctaagatttttttcaaaaaaggcattttagacaagaactaattactacaactaaaatctactaactttggtttttatgtatacatttacgagagtctacacaaaaaaaatacataaaagaagaagtagtaaataattaaaacgtaaaaaaaaagacttttcaagaaggcagaagagtagcactgttgtgcgattgtaaaatcgtcttgattgttgtttatcccactgataagcctacccttttcaaaacttgttctaagtttaaattctatttttgaacatgacatacagtataaggctttttaaagatatcatcttgattttgtttcttccacagggaagccctatcctttcttacataatctttgttagataattttttattattttcaaaaaaaataaaattgaacataagttttctcaaagtaatatgttctaacaataataaacataatatcatttttttgttttaaactataaaggactaacatggtaaaaagttgcaatatataaatgataatttaaactaaaaattagaatatggtaactttttcttcaacaacatgccacattcggctacatgtccactaggaagtgttattatagaatcgttaatgttgggtacgcttatgaaattatcaatgtttgcttaaatctatgcttagaaaattaccaatattaccttaaaactatatttacgaatgaccaatattgcttagaactatgcttatgaaattaccaatattttcttaaaacttaaacacaaaactctttaacaaaaaaaactttatttttatttttatttttttggcaaaaaaaaaaactttatttataaagtgaaagtctccagataattttgaatttcatttttccagtttttatttagaataatttttcttcatttacaaaataaaagaaaaccctagggtttagggtttagggtttaggaaaaagcgatgatatattaattgttatgaaatgtttttttaaaaatagttaaccaaacatttttttaaagagagtttagtttcacaaggcatttgtaaattagagtaattatcaataaaaatggaagacaatctaattattatttagcaaaaactatatttaggaaaattagttaaagtttagaaatatatcatcatagtgtcaaactaattaaaattatttaattttgtgatatacgtgatcatataattttatgaatatttaatattatgatacatgtaactcagtaaacctaaatttagaagaaaagtcaaaataatcataaccaatttagattcaacttctacttttgttccaagaaaaaaacacatggtttgttttgtgggatactaatgacatctatcaaaatctatgaaaccaaatctaga

SEQ ID NO: 15 - DNA, 755 bp, Cauliflower Mosaic Virus
cctgcaggtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagga

SEQ ID NO: 16 - DNA, 210 bp, Cauliflower Mosaic Virus
gtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtga

SEQ ID NO: 17 - DNA, 29 bp, Artificial Sequence
  Primer Erg5858link
atgaattctgtctgcagtgtcaatctcta

SEQ ID NO: 18 - DNA, 32 bp, Artificial Sequence
  Primer ER6000Bam.rc
tcaggatcctatgatccatcaagaaaaggagg

SEQ ID NO: 19 - DNA, 32 bp, Artificial Sequence
  Primer ER-6000link.rc
tcaggatccgctgatccatcaagaaaaggagg

SEQ ID NO: 20 - DNA, 29 bp, Artificial Sequence
  Primer myc-5
gaagatctcgagttcggtgaacaaaagtt

SEQ ID NO: 21 - DNA, 31 bp, Artificial Sequence
  Primer myc-3
cgggatccttaccctagctttccgttcaagt

SEQ ID NO: 22 - protein, 45 aa, Artificial Sequence
  Primer triple c-myc
ADLEFGEQKLISEEDLNGEQKLISEEDLNGEQKLISEEDLNGKLG

SEQ ID NO: 23 - DNA, 21 bp, Artificial Sequence
  Primer ERg1761
gtatatctaaaaacgcagtcg

SEQ ID NO: 24 - DNA, 24 bp, Artificial Sequence
  Primer ERg2339rc
caacaacattgaaggtgacatttt

SEQ ID NO: 25 - DNA, 30 bp, Artificial Sequence
  Primer ERLRRab5
cggaattctcattcaaagatgtgaacaatg

SEQ ID NO: 26 - DNA, 30 bp, Artificial Sequence
  Primer ERLRRab3
cgtctagactatgacactcgtacagttcga

SEQ ID NO: 27 - DNA, 19 bp, Artificial Sequence
  Primer ERK7
cacagagacgtgaagtcgt

SEQ ID NO: 28 - DNA, 24 bp, Artificial Sequence
  Primer ERg7361rc
agcttaacgcaacgaaaagatacc

SEQ ID NO: 29 - DNA, 22 bp, Artificial Sequence
  Primer ERg5022
cttgagtagaaatcatataact

SEQ ID NO: 30 - DNA, 21 bp, Artificial Sequence
  Primer ERg5757rc
tgacacggtgagtttagccaa

SEQ ID NO: 31 - DNA, 22 bp, Artificial Sequence
  Primer ERg5022
cttgagtagaaatcatataact

SEQ ID NO: 32 - DNA, 20 bp, Artificial Sequence
  Primer ACT2-1
gccatccaagctgttctctc

SEQ ID NO: 33 - DNA, 21 bp, Artificial Sequence
  Primer ACT2-2
gctcgtagtcaacagcaacaa

SEQ ID NO: 34 - DNA, 41 bp, Artificial Sequence
  Primer ERK4
cggaattcactagtaccatggacaaaccagtaacttattcg

SEQ ID NO: 35 - DNA, 34 bp, Artificial Sequence
  Primer ER3rc
cgggatccactagtgcataatactttacatgaga

SEQ ID NO: 36 - DNA, 38 bp, Artificial Sequence
  Primer ERKI3/K676E
ctgtgggttgtgagagtaaagccgttcaatcgcaaccg

SEQ ID NO: 37 - DNA, 26 bp, Artificial Sequence
  Primer ERKI5/K676E
ttgaacggctttactctcacaaccca

SEQ ID NO: 38 - DNA, 40 bp, Artificial Sequence
  Primer ERCodeC3
cgggatccactagtctactcactgttctgagaaataactt

SEQ ID NO: 39 - DNA, 20 bp, Artificial Sequence
  Primer ERL1.14coding
ggctctttcagcaacttagt

SEQ ID NO: 40 - DNA, 24 bp, Artificial Sequence
  Primer ERL1g6054rc
cttctgcatcaggattcctaactt

SEQ ID NO: 41 - DNA, 20 bp, Artificial Sequence
  Primer ERL2.3coding
ggcgataaaggcttcattca

SEQ ID NO: 42 - DNA, 24 bp, Artificial Sequence
  Primer ERL2g5352rc
ttgtatctgaagagtggctctcac

SEQ ID NO: 43 - DNA, 18 bp, Artificial Sequence
  Primer Elk1-300rc
tccatataacagattctc

SEQ ID NO: 44 - DNA, 18 bp, Artificial Sequence
  Primer Ekl2-300.rc
tccaaacaacaaattggt

SEQ ID NO: 45 - DNA, 21 bp, Artificial Sequence
  Primer Elk1-185rc
cgtaggtctccaatagctgga

SEQ ID NO: 46 - DNA, 21 bp, Artificial Sequence
  Primer Elk2-185rc
atcaaatctccaagggcagat

SEQ ID NO: 47 - DNA, 20 bp, Artificial Sequence
  Primer ERg4359
caacaatgatctggaaggac

SEQ ID NO: 48 - DNA, 20 bp, Artificial Sequence
  Primer ERL1g2846
tatcccaccgatacttggca

SEQ ID NO: 49 - DNA, 20 bp, Artificial Sequence
  Primer ERL1g4411rc
ccggagagattgttgaagga

SEQ ID NO: 50 - DNA, 21 bp, Artificial Sequence
  Primer ERL2g3085
ctgtctggcaacaatttctca

SEQ ID NO: 51 - DNA, 20 bp, Artificial Sequence
  Primer ERL2g4254rc
agccatgtccatgtgaagaa

SEQ ID NO: 52 - DNA, 19 bp, Artificial Sequence
  Primer 5′ant-1
gcccaacacgactacaaac

SEQ ID NO: 53 - DNA, 22 bp, Artificial Sequence
  Primer ANT1600rc
tcatatctaccagtccatctat

SEQ ID NO: 54 - DNA, 22 bp, Artificial Sequence
  Primer STM781
tggagatccatcataacgaaat

SEQ ID NO: 55 - DNA, 22 bp, Artificial Sequence
  Primer STM2354rc
gacccattattgttcctatcaa

SEQ ID NO: 56 - DNA, 29 bp, Artificial Sequence
  Primer U3WUS5
gtgaacaaaagtcgaatcaaacacacatg

SEQ ID NO: 57 - DNA, 24 bp, Artificial Sequence
  Primer U34WUS3rc
gctagttcagacgtagctcaagag

SEQ ID NO: 58 - DNA, 19 bp, Artificial Sequence
  Primer BP681
gctcctcaagaatcaatca

SEQ ID NO: 59 - DNA, 26 bp, Artificial Sequence
  Primer BP3100rc
aagctataagtagcaaactgatgtag

SEQ ID NO: 60 - DNA, 20 bp, Artificial Sequence
  Primer CycD2.501
atggctgagaatcttgcttg

SEQ ID NO: 61 - DNA, 21 bp, Artificial Sequence
  Primer CycD2.801rc
atttagaatccaatcaagagc

SEQ ID NO: 62 - DNA, 20 bp, Artificial Sequence
  Primer CycD3.501
tggatttagaagaggaggaa

SEQ ID NO: 63 - DNA, 21 bp, Artificial Sequence
  Primer CycD3.935rc
aaggaacacggatctcttcaa

SEQ ID NO: 64 - DNA, 29 bp, Artificial Sequence
  Primer ERL1g3036
gtcacgtctcagctatttgtaagcttgtt

SEQ ID NO: 65 - DNA, 30 bp, Artificial Sequence
  Primer ERL1-3endrc
cgtctagattatatgctacttttggagatg

SEQ ID NO: 66 - DNA, 20 bp, Artificial Sequence
  Primer ERL2g2166
gcctattccaccaatacttg

SEQ ID NO: 67 - DNA, 30 bp, Artificial Sequence
  Primer ERL2-3endrc
cgtctagattataagctacttttggagata

SEQ ID NO: 68 - DNA, 28 bp, Artificial Sequence
  Primer ERL1-5end
gctctagaaatgaaggagaagatgcagc

SEQ ID NO: 69 - DNA, 28 bp, Artificial Sequence
  Primer ERL2-5end
gctctagagatgagaaggatagagacca

SEQ ID NO: 70 - DNA, 29 bp, Artificial Sequence
  Primer ERL2g3182rc
acaaatctgagagagttaatgcaaagcag

SEQ ID NO: 71 - DNA, 30 bp, Artificial Sequence
  Primer ERL1g-3680link
aggaattcacaccaataaaaatacacagca

SEQ ID NO: 72 - DNA, 40 bp, Artificial Sequence
  Primer ERL1g403linkrc
aggaattcgtcgacttcttcttattcttctttccttttgg

SEQ ID NO: 73 - DNA, 31 bp, Artificial Sequence
  Primer ERL2g-4364link
aggaattcgtgattaggagacgaggtagata

SEQ ID NO: 74 - DNA, 37 bp, Artificial Sequence
  Primer ERL2g4linkrc
aggaattcgtcgaccttcttcttcttcttcctcaaga

SEQ ID NO: 75 - DNA, 29 bp, Artificial Sequence
  Primer JL-202
cattttataataacgctgcggacatctac

SEQ ID NO: 76 - DNA, 30 bp, Artificial Sequence
  Primer ERLK765
tacccaatacttagctctgggcttgttctt

SEQ ID NO: 77 - DNA, 29 bp, Artificial Sequence
  Primer ERLK6137rc
tccttccaatcagcattactatcttcctt

SEQ ID NO: 78 - DNA, 29 bp, Artificial Sequence
  Primer ERTJ70
aacaacgaaggttctagctctttcaaaat

SEQ ID NO: 79 - DNA, 29 bp, Artificial Sequence
  Primer ERTJ5855rc
acaagtgaacaacacatctccatcaatta

SEQ ID NO: 80 - DNA, 22 bp, Artificial Sequence
  Primer ERg2248
aagaagtcatctaaagatgtga

SEQ ID NO: 81 - DNA, 22 bp, Artificial Sequence
  Primer er-105
agctgactatacccgatactga

SEQ ID NO: 82 - DNA, 39 bp, Artificial Sequence
  Primer Ercode5
cggaattcactagtaccatggctctgtttagagatattg

SEQ ID NO: 83 - DNA, 22 bp, Artificial Sequence
  Primer ERg3476rc
atacaaaacctggaaggcagtg

SEQ ID NO: 84 - DNA, 36 bp, Artificial Sequence
  Primer ERL1_433XbPc
gctctagacatgttggagaagatgcagcgaatggtt

SEQ ID NO: 85 - DNA, 34 bp, Artificial Sequence
  Primer ERL1_4828StopXbRC
gctctagactaggagccttgtagaatcttcttct

SEQ ID NO: 86 - protein, 613 aa, Oryza Sativa
MTTTTTTRLLLAAILLAVAAADDDGQTLLEIKKSFRNVDNVLYDWAGDGAPRRYCSWRGVLCDNVTFAVAALNLSGLNLGGEISPAIGNLKSVESIDLKSNELSGQIPDEIGDCTSLKTLDLSSNNLGGDIPFSISKLKHLENLILKNNQLVGMIPSTLSQLPNLKILDLAQNKLNGEIPRLIYWNEVLQYLGLRSNNLEGSLSPEMCQLTGLWYFDVKNNSLTGIIPDTIGNCTSFQVLDLSYNRLTGEIPFNIGFLQVATLSLQGNNFSGPIPSVIGLMQALAVLDLSFNQLSGPIPSILGNLTYTEKLYLQGNRLTGSIPPELGNMSTLHYLELNDNQLTGFIPPELGKLTGLFDLNLANNNLEGPIPDNISSCMNLISFNAYGNKLNGTVPRSLHKLESITYLNLSSNYLSGAIPIELAKMKNLDTLDLSCNMVAGPIPSAIGSLEHLLRLNFSNNNLVGYIPAEFGNLRSIMEIDLSSNHLGGLIPQEVGMLQNLILLKLESNNITGDVSSLINCFSLNVLNVSYNNLAGIVPTDNNFSRFSPDSFLGNPGLCGYWLGSSCYSTSHVQRSSVSRSAILGIAVAGLVILLMILAAACWPHWAQVPKDVS

SEQ ID NO: 87 - protein, 611 aa, Oryza Sativa
MTPAPAAASYRALVALLLVAVAVADDGSTLLEIKKSFRNVDNVLYDWAGGDYCSWRGVLCDNVTFAVAALNLSGLNLGGEISPAVGRLKGIVSIDLKSNGLSGQIPDEIGDCSSLKTLDLSFNSLDGDIPFSVSKLKHIESLILKNNQLIGVIPSTLSQLPNLKILDLAQNKLSGEIPRLIYWNEVLQYLGLRGNNLEGSISPDICQLTGLWYFDVKNNSLTGPIPETIGNCTSFQVLDLSYNKLSGSIPFNIGFLQVATLSLQGNMFTGPIPSVIGLMQALAVLDLSYNQLSGPIPSILGNLTYTEKLYMQGNKLTGPIPPELGNMSTLHYLELNDNQLSGFIPPEFGKLTGLFDLNLANNNFEGPIPDNISSCVNLNSFNAYGNRLNGTIPPSLHKLESMTYLNLSSNFLSGSIPIELSRINNLDTLDLSCNMITGPIPSTIGSLEHLLRLNLSNNGLVGFIPAEIGNLRSIMEIDMSNNHLGGLIPQELGMLQNLMLLNLKNNNITGDVSSLMNCFSLNILNVSYNNLAGVVPTDNNFSRFSPDSFLGNPGLCGYWLGSSCRSSGHQQKPLISKAAILGIAVGGLVILLMILVAVCRPHSPPVFKDVS

SEQ ID NO: 88 - protein, 621 aa, Oryza Sativa
MAAARAPWLWWWVVVVVGVAVAEAASGGGGGGDGEGKALMGVKAGFGNAANALVDWDGGADHCAWRGVTCDNASFAVLALNLSNLNLGGEISPAIGELKNLQFVDLKGNKLTGQIPDEIGDCISLKYLDLSGNLLYGDIPFSISKLKQLEELILKNNQLTGPIPSTLSQIPNLKTLDLAQNQLTGDIPRLIYWNEVLQYLGLRGNSLTGTLSPDMCQLTGLWYFDVRGNNLTGTIPESIGNCTSFEILDISYNQISGEIPYNIGFLQVATLSLQGNRLTGKIPDVIGLMQALAVLDLSENELVGPIPSILGNLSYTGKLYLHGNKLTGVIPPELGNMSKLSYLQLNDNELVGTIPAELGKLEELFELNLANNNLQGPIPANISSCTALNKFNVYGNKLNGSIPAGFQKLESLTYLNLSSNNFKGNIPSELGHIINLDTLDLSYNEFSGPVPATIGDLEHLLELNLSKNHLDGPVPAEFGNLRSVQVIDMSNNNLSGSLPEELGQLQNLDSLILNNNNLVGEIPAQLANCFSLNNLNLSYNNLSGHVPMAKNFSKFPMESFLGNPLLHVYCQDSSCGHSHGQRVNISKTAIACIILGFIILLCVLLLAIYKTNQPQPLVKGS

(19) United States
(12) Patent Application Publication
(10) Pub. No.: US 2005/0223428 A1
(43) Pub. Date: Oct. 6, 2005

(54) Methods for modulating plant growth

(76) Inventors: Keiko U. Torii, Seattle, WA (US); Elena D. Shpak, Beaverton, OR (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(21) Appl. No.: 11/027,304

(22) Filed: Dec. 30, 2004

Related U.S. Application Data
(60) Provisional application No. 60/558,529, filed on Apr. 1, 2004.

(51) Int. Cl. A01H 1/00; C12N 15/82

(52) U.S. Cl. 800/287

(57) ABSTRACT

The invention provides methods for modulating plant height and organ shape, comprising the step of expressing a transgene in a plant, wherein the transgene encodes an ERECTA-like protein lacking an active kinase domain and wherein expression of the transgene modulates plant height or organ shape. The invention also provides methods for for enhancing the yield of a crop plant, transgenic plants comprising a gene encoding an ERECTA-like protein, vectors encoding ERECTA-like proteins and host cells and/or cell cultures comprising these vectors, and isolated nucleic acid sequences.

### CROSS-REFERENCE TO RELATED APPLICATION

This application claims the benefit of U.S. Provisional Application No. 60/558,529, filed Apr. 1, 2004.

### STATEMENT OF GOVERNMENT LICENSE RIGHTS

The U.S. Government has a paid-up license in this invention and the right in limited circumstances to require the patent owner to license others on reasonable terms as provided for by the terms of DE-FG02-03ER15448 awarded by the U.S. Department of Energy.

### FIELD OF THE INVENTION

The present invention relates to methods for modulating plant growth and organogenesis using dominant-negative receptor-like kinases.

### BACKGROUND OF THE INVENTION

The bodies of plants are built by a reiterative formation of the shoot system, which consists of a node bearing a lateral organ (e.g., a leaf) and an internode (e.g., a stem). Both lateral organs and internodes are generated from distinct domains of the shoot apical meristem, in which continual cell proliferation and differentiation take place. The basic pattern and identity of the shoots are determined at the shoot apical meristem, and their final size and shape, which contribute to the diversity of the plant form, are elaborated by localized cell division and cell expansion during plant organ morphogenesis. It is desirable to manipulate the growth and morphogenesis of a plant, for example in order to produce dwarf plants. There are several advantages associated with dwarf plants. For example, dwarf crop plants direct more energy and nutrients into making seeds or grain than into making vegetative tissue (e.g., stalks). It is also possible to grow more dwarf plants per unit area of land, which may also increase the yield of crops.

Although mechanisms for plant cell division and expansion have been studied extensively, little is known about how these two cellular processes are integrated in the context of whole plant growth and development. Increasing evidence supports the view that, although cell proliferation and cellular growth are an instrumental process of organ growth, the final size and forms of organs are governed by intrinsic mechanisms that monitor and balance the number and size of cells within the context of developmental programs (Conlon & Raff (1999) Cell 96:235-44; Day & Lawrence (2000) Development 127:2977-87; Mizukami (2001) Curr. Opin. Plant Biol. 4:533-9; Nijout (2003) Dev. Biol. 261:1-9; Potter & Xu (2001) Curr. Opin. Genet. Dev. 11:279-86). Experimental manipulation of cell cycle regulators, for example, does not always lead to altered organ size, as defects in cell number are compensated by alteration of cell size (Hemerly et al. (1995) EMBO J. 14:3925-36; Neufeld et al. (1988) Cell 93:1183-93). Similarly, alteration of cellular growth has been shown to be compensated by changes in cell number (Johnston et al. (1999) Cell 98:779-90; Jones et al. (1998) Science 282:1114-7). For instance, transgenic tobacco plants overexpressing a dominant-negative form of Cdc2 produced nearly normal organs, both in overall size and patterning, despite the fact that the transgene severely compromised cell division (Trotochaud et al. (1999) Plant Cell 11:393-405). Although overexpression of the cyclin kinase inhibitor ICK1 in Arabidopsis plants resulted in small organs, the cells that made up such small organs were much larger than control cells (Wang et al. (2000) Plant J. 24:613-23). These findings imply that plants may somehow monitor and balance the activity of cell division and cell expansion to retain a stable organ size.

The molecular basis of the cell-to-cell signaling that coordinates cell division and expansion during plant organogenesis is not clear. One candidate gene is Arabidopsis ERECTA, which regulates organ shape and inflorescence architecture. Loss of-function erecta mutations confer a compact inflorescence with short internodes and clustered flower buds, short pedicels, round flowers, and short, blunt siliques (Bowman (1993) Arabidopsis: An Atlas of Morphology and Development (Springer-Verlag, New York); Torii et al. (1996) Plant Cell 8:735-46). Despite these defects, the erecta mutation does not affect organ identity, polarity, or tissue organization.

There is a need for methods for modulate the growth or form of a plant, particularly for producing dwarf plants. The present invention addresses this need and others.

### SUMMARY OF THE INVENTION

In one aspect, the invention provides a method for modulating plant height and organ shape, comprising the step of expressing a transgene in a plant, wherein the transgene encodes an ERECTA-like protein lacking an active kinase domain and wherein expression of the transgene modulates plant height or organ shape. Suitable ERECTA-like proteins are proteins belonging to the ERECTA family of proteins, including, but not limited to the Arabidopsis proteins ERECTA, ERL1, and ERL2, and the rice proteins ERa, ERb, and ERc.

In some embodiments, the ERECTA-like protein has an amino acid sequence that is at least about 60% identical (such as at least 70%, at least 80%, at least 85%, at least 90%, at least 95%, or at least 99%) to at least one of the sequences provided in SEQ ID NOs:2, 4, 6, 8, 10, 12, 86, 87, and 88. In some embodiments, the ERECTA-like protein lacking an active kinase domain has an amino acid sequence that is at least about 60% identical (such as at least 70%, at least 80%, at least 85%, at least 90%, at least 95%, or at least 99%) to at least one of the sequences provided in SEQ ID NOs:4, 10, 12, and 86-88. In some embodiments, the ERECTA-like protein lacking an active kinase domain comprises the amino acid sequence provided in one of SEQ ID NOs:4, 10, 12, and 86-88.

Any plant may be used in the practice of the methods for modulating plant height and organ shape. Suitable plants include, but are not limited to, crop plants plant such as rice or canola. In some embodiments, the transgene is expressed in the shoot apical meristem of the plant. In some embodiments, expressing the transgene in the plant produces a dwarf plant.

Another aspect of the invention provides methods for enhancing the yield of a crop plant, comprising the steps of: (a) introducing a transgene into a crop plant, wherein the transgene encodes an ERECTA-like protein lacking an active kinase domain and wherein expression of the transgene enhances the yield of the crop plant; and (b) growing the transgenic crop plant under conditions in which the transgene is expressed to enhance the yield of the crop plant. Suitable transgenes encoding an ERECTA-like protein lacking an active kinase domain are as described above. In some embodiments, the crop plant is a rice plant or a canola plant.

A further aspect of the invention provides transgenic plants, such as transgenic crop plants, comprising a gene encoding an ERECTA-like protein lacking an active kinase domain. Suitable transgenes encoding an ERECTA-like protein lacking an active kinase domain are as described above.

Other aspects of the invention provide vectors comprising a nucleic acid sequence encoding an ERECTA-like protein lacking an active kinase domain, and host cells or cell cultures comprising such vectors.

The invention is useful for producing transgenic plants whose plant height and organ shape is altered, for example for producing a dwarf plants that direct relatively more resources into making seeds and grain than normal-size plants.

### DETAILED DESCRIPTION OF THE PREFERRED EMBODIMENT

Unless specifically defined herein, all terms used herein have the same meaning as they would to one skilled in the art of the present invention.

In one aspect, the invention provides methods for modulating plant height and organ shape. The methods comprise the step of expressing a transgene in a plant, wherein the transgene encodes an ERECTA-like protein lacking an active kinase domain and wherein expression of the transgene modulates plant height or organ shape.

As used herein, the term “ERECTA-like protein” refers to a protein with structural and functional similarities to the ERECTA-family of proteins. The prototypical member of the ERECTA-family of proteins is the ERECTA protein (SEQ ID NO:2) encoded by the Arabidopsis ERECTA gene (cDNA sequence provided as SEQ ID NO:1). Arabidopsis ecotype Landsberg erecta (Ler) carries a mutation in the ERECTA locus, which confers compact inflorescence with tight flower clusters at the tip, short internodes, short pedicels, and short and blunt siliques (Torii et al. (1996) Plant Cell 8:735-46). Phenotypic comparison of 21 erecta alleles revealed that ERECTA regulates plant size in a quantitative manner, as the degree of allelic severity correlates with the degree of plant height and organ size (Torii et al. (1996) Plant Cell 8:735-46; Lease et al. (2001) New Phytol. 151:133-44; Torii et al. (2003) in Morphogenesis and Patterning of Biological Systems (ed. T. Sekimura, Tokyo, Japan: Springer-Verlag) pp. 153-64). These phenotypes are largely attributable to reduced cell numbers in the cortex cell files, as described in EXAMPLE 1. ERECTA is highly expressed in the shoot apical meristem (SAM) and developing lateral organs, where cells are actively dividing (Yokoyama (1998) Plant J. 15:301-10).

ERECTA encodes a leucine-rich repeat receptor-like kinase (LRR-RLK) with 20 consecutive leucine-rich repeats (LRRs) and functional Ser/Thr kinase activity (Torii et al. (1996) Plant Cell 8:735-46; Lease et al. (2001) New Phytol. 151:133-44; SEQ ID NO:2). The LRR-RLKs constitute the largest subfamily of plant RLKs and possess a structural organization similar to that of animal receptor kinases (Torii (2000) Curr. Opin. Plant Biol. 3:361-7; Shiu & Bleecker (2001) Proc. Natl. Acad. Sci. U.S.A. 98:10763-8; Torii et al. (2003) in Morphogenesis and Patterning of Biological Systems (ed. T. Sekimura, Tokyo, Japan: Springer-Verlag) pp. 153-64). Unlike animal receptor kinases, the kinase domain of some plant LRR-RLKs appears partially dispensable. For instance, two CLAVATA1 alleles that truncate the entire kinase domain, clv1-6 and clv1-7 have the weakest phenotypes (Clark et al. (1997) Cell 89:575-85). Expression of Xa21D, a naturally-occurring variant of the rice disease resistance gene that lacks the entire kinase domain, confers partial resistance to a full-spectrum of pathogens (Wang et al. (1998) Plant Cell 10:765-79). In contrast, expression of a transgene (SEQ ID NO:3) encoding a truncated ERECTA lacking the cytoplasmic kinase domain (delta-Kinase ERECTA, SEQ ID NO:4) results in an inhibition of normal ERECTA function and confers dominant-negative effects in Arabidopsis organ growth and internodal elongation, as described in EXAMPLE 1. Thus, expression of delta-Kinase ERECTA (SEQ ID NO:4) produces phenotypes that are identical to a loss-of-function erecta mutant, including compact inflorescence, short internodes, and short and round flowers and fruits.

The family of ERECTA-like proteins also includes functional paralogs, orthologs, and homologs of ERECTA. For example, the family of ERECTA-like proteins includes Arabidopsis paralogs of ERECTA such as proteins encoded by ERL1 (cDNA sequence provided as SEQ ID NO:5) and ERL2 (cDNA sequence provided as SEQ ID NO:7). The predicted proteins encoded by ERL1 and ERL2, ERL1 (SEQ ID NO:6) and ERL2 (SEQ ID NO:8), respectively, share high overall sequence identity to ERECTA (60% identity, 72% similarity), as described in EXAMPLE 2. Loss-of-function mutations in ERL1 and ERL2 each enhance the erecta mutant phenotype, as described in EXAMPLE 2. The family of ERECTA-like proteins also includes ERECTA proteins form other plant species, such as, for example, the rice (Oryza sativa) proteins encoded by ERa (Temporary Gene ID: 9630.t05117, The Institute of Genomic Research, http://www.tigr.org), ERb (accession number AK073793, Temporary Gene ID: 9634.t00945, The Institute of Genomic Research, http://www.tigr.org), and ERc (accession numbers XM_{—}550586, AK064052, AY332474, XM_{—}493694), and the ERECTA-like protein from Sorghum bicolor (accession number AF466166).

Also included within the definition of ERECTA-like proteins useful in the present invention are proteins that are substantially identical to ERECTA, ERL1, or ERL2 (SEQ ID NOS:2, 6, or 8, respectively), or that are encoded by nucleic acid sequences that are substantially identical to the nucleic acid sequences encoding ERECTA, ERL1, or ELR2 (SEQ ID NOS: 1, 5, or 7, respectively). As used herein, the term “substantial identity” in the context of nucleic acid sequences means that a nucleic acid molecule has at least 70% sequence identity, preferably at least 80%, more preferably at least 90%, and most preferably at least 95%, compared to a reference sequence using one of the alignment programs described below using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. The term “substantial identity” in the context of a peptide indicates that a peptide has at least 70% sequence identity to a reference sequence, preferably 80%, more preferably 85%, most preferably at least 90% or at least 95% sequence identity to the reference sequence over a specified comparison window.

As used herein, the term “reference sequence” refers to a defined sequence used as a basis for sequence comparison. A reference sequence may be a subset or the entirety of a specified sequence; for example, as a segment of a full-length cDNA or gene sequence, or the complete cDNA or gene sequence.

As used herein, “comparison window” refers to a contiguous and specified segment of a sequence, wherein the sequence in the comparison window may comprise additions or deletions (i.e., gaps) compared to the reference sequence (which does not comprise additions or deletions) for optimal alignment of the two sequences. Generally, the comparison window is at least 20 contiguous nucleotides or amino acids in length, and optionally can be 30, 40, 50, 100, or longer. Those of skill in the art understand that to avoid a high similarity to a reference sequence due to inclusion of gaps in the sequence a gap penalty is typically introduced and is subtracted from the number of matches.

Methods of alignment of sequences for comparison are well known in the art. Thus, the determination of percent sequence identity between any two sequences can be accomplished using a mathematical algorithm. Non-limiting examples of such mathematical algorithms are the algorithm of Myers & Miller (1988) CABIOS 4:11-17; the local homology algorithm of Smith et al. (1981) Adv. Appl. Math. 2:482; the homology alignment algorithm of Needleman & Wunsch (1970) J. Mol. Biol. 48:443-53; the search-for-similarity-method of Pearson & Lipman (1988) Proc. Natl. Acad. Sci. U.S.A. 85:2444-8; the algorithm of Karlin & Altschul (1990) Proc. Natl. Acad. Sci. U.S.A. 87:2264-8, modified as in Karlin & Altschul (1993) Proc. Natl. Acad. Sci. U.S.A. 90:5873-7.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the Wisconsin Genetics Software Package, Version 8 (available from Genetics Computer Group (GCG programs (Accelrys, Inc., San Diego, Calif.)). Alignments using these programs can be performed using the default parameters. The CLUSTAL program is well described by Higgins et al. (1988) Gene 73:237-244 (1988); Higgins et al. (1989) CABIOS 5:151-153; Corpet et al. (1988) Nucleic Acids Res. 16:10881-90; Huang et al. (1992) CABIOS 8:155-65; and Pearson et al. (1994) Meth. Mol. Biol. 24:307-331. The ALIGN program is based on the algorithm of Myers & Miller (1988) CABIOS 4:11-17. A PAM120 weight residue table, a gap length penalty of 12, and a gap penalty of 4 can be used with the ALIGN program when comparing amino acid sequences. The BLAST programs of Altschul et al (1990) J. Mol. Biol. 215:403 are based on the algorithm of Karlin & Altschul (1990) Proc. Natl. Acad. Sci. U.S.A. 87:2264-8. BLAST nucleotide searches can be performed with the BLASTN program, score=100, wordlength=12, to obtain nucleotide sequences homologous to a nucleotide sequence encoding a protein of the invention. BLAST protein searches can be performed with the BLASTX program, score=50, wordlength=3, to obtain amino acid sequences homologous to a protein or polypeptide of the invention. To obtain gapped alignments for comparison purposes, Gapped BLAST (in BLAST 2.0) can be used, as described in Altschul et al. (1997) Nucleic Acids Res. 25:3389. Alternatively, PSI-BLAST (in BLAST 2.0) can be used to perform an iterated search that detects distant relationships between molecules, as described in Altschul et al. (1997) Nucleic Acids Res. 25:3389. When utilizing BLAST, Gapped BLAST, PSI-BLAST, the default parameters of the respective programs (e.g., BLASTN for nucleotide sequences, BLASTX for proteins) can be used. Alignment may also be performed manually by inspection.

Unless otherwise stated, sequence identity/similarity values provided herein refer to the value obtained using GAP (e.g., GCG programs (Accelrys, Inc., San Diego, Calif.) version 10) using the following parameters: percent identity using GAP Weight of 50 and Length Weight of 3; percent similarity using Gap Weight of 12 and Length Weight of 4, or any equivalent program. The term “equivalent program” refers to any sequence comparison program that, for any two sequences in question, generates an alignment having identical nucleotide or amino acid residue matches and an identical percent sequence identity when compared to the corresponding alignment generated by GAP.

GAP uses the algorithm of Needleman & Wunsch (1970) J. Mol. Biol. 48:443-53, to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases. GAP must make a profit of gap creation penalty number of matches for each gap it inserts. If a gap extension penalty greater than zero is chosen, GAP must, in addition, make a profit for each gap inserted of the length of the gap times the gap extension penalty. Default gap creation penalty values and gap extension penalty values in Version 10 of the Wisconsin Genetics Software Package for protein sequences are 8 and 2, respectively. For nucleotide sequences the default gap creation penalty is 50 while the default gap extension penalty is 3. The gap creation and gap extension penalties can be expressed as an integer selected from the group of integers consisting of from 0 to 200. Thus, for example, the gap creation and gap extension penalties can be 0, 1, 2, 3, 4, 5, 6, 7, 8, 9, 10, 15, 20, 25, 30, 35, 40, 45, 50, 55, 60, 65 or greater.

GAP presents one member of the family of best alignments. There may be many members of this family, but no other member has a better quality. GAP displays four figures of merit for alignments: Quality, Ratio, Identity, and Similarity. The Quality is the metric maximized in order to align the sequences. Ratio is the quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the Wisconsin Genetics Software Package is BLOSUM62 (see Henikoff & Henikoff (1989) Proc. Natl. Acad. Sci. U.S.A. 89:10915).

As used herein, “sequence identity” or “identity” in the context of two nucleic acid or polypeptide sequences refers to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, for example as implemented in the program PC/GENE (Intelligenetics, Mountain View, Calif.). Conservative substitution tables providing functionally similar amino acids are well known in the art (Henikoff & Henikoff (1992) Proc. Natl. Acad. Sci. U.S.A. 89:10915-9).

As used herein, “percentage of sequence identity” means the value determined by comparing two optimally aligned sequences over a comparison window, wherein the portion of the sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

Another indication that nucleic acid sequences are substantially identical is if two molecules hybridize to each other under stringent conditions. Generally, stringent conditions are selected to be about 5° C. lower than the thermal melting point (Tₘ) for the specific sequence at a defined ionic strength and pH. However, stringent conditions encompass temperatures in the range of about 1° C. to about 20° C. lower than the Tₘ, depending upon the desired degree of stringency as otherwise qualified herein. Nucleic acid molecules that do not hybridize to each other under stringent conditions are still substantially identical if the polypeptides they encode are substantially identical. This may occur, for example, when a copy of a nucleic acid molecule is created using the maximum codon degeneracy permitted by the genetic code.

The nucleic acid sequences coding for proteins that are useful for modulating the growth or form of a plant according to the methods of the invention, such as nucleic acid sequences coding for ERECTA, ERL1, or ERL2 (SEQ ID NOS: 1, 5, or 7, respectively) may be used to isolate corresponding sequences from other organisms, particularly other plants, such as monocots. In this manner, methods such as PCR, hybridization, and the like can be used to identify such sequences based on their sequence similarity to the sequences set forth herein. Such sequences include sequences that are orthologs. By “orthologs” is intended genes derived from a common ancestral gene and which are found in different species as a result of speciation. Genes found in different species are considered orthologs when their nucleotide sequences and/or their encoded protein sequences share substantial identity as defined elsewhere herein. Functions of orthologs are often highly conserved among species. Thus, the use of isolated sequences that encode for an ERECTA-like protein and which hybridize under stringent conditions to the sequences coding for an ERECTA-like protein, or to fragments thereof, is encompassed by the present invention.

In a PCR approach, oligonucleotide primers can be designed for use in PCR reactions to amplify corresponding DNA sequences from cDNA or genomic DNA extracted from any plant of interest. Methods for designing PCR primers and PCR cloning are generally known in the art and are disclosed, for example, in Sambrook et al. (1989) Molecular Cloning: A Laboratory Manual (2d ed., Cold Spring Harbor Laboratory Press, Plainview, N.Y.); Innis et al., eds. (1990) PCR Protocols: A Guide to Methods and Applications (Academic Press, New York); Innis & Gelfand, eds. (1995) PCR Strategies (Academic Press, New York); and Innis & Gelfand, eds. (1999) PCR Methods Manual (Academic Press, New York). Known methods of PCR include, but are not limited to, methods using paired primers, nested primers, single specific primers, degenerate primers, gene-specific primers, vector-specific primers, partially-mismatched primers, and the like.

In hybridization techniques, all or part of a known nucleotide sequence is used as a probe that selectively hybridizes to other corresponding nucleotide sequences present in a population of cloned genomic DNA fragments or cDNA fragments (i.e., genomic or cDNA libraries) from a chosen organism. The hybridization probes may be genomic DNA fragments, cDNA fragments, RNA fragments, or other oligonucleotides, and may be labeled with a detectable marker. Thus, for example, probes for hybridization can be made by labeling synthetic oligonucleotides based on the sequences coding for ERECTA-like proteins. Methods for preparation of probes for hybridization and for construction of cDNA and genomic libraries are generally known in the art and are disclosed in Sambrook et al. (1989) Molecular Cloning: A Laboratory Manual (2d ed., Cold Spring Harbor Laboratory Press, Plainview, N.Y.).

For example, the entire sequence coding for the Arabidopsis ERECTA, ERL1, or ERL2 (SEQ ID NO:1, SEQ ID NO:5, and SEQ ID NO:7, respectively), or one or more portions thereof, may be used as a probe capable of specifically hybridizing to corresponding genomic sequences and messenger RNAs from other plant species. To achieve specific hybridization under a variety of conditions, such probes include sequences coding for ERECTA-like proteins and are preferably at least about 10 -nucleotides in length, and most preferably at least about 15, about 20 or about 50 nucleotides in length. Such probes may be used to amplify corresponding sequences from a chosen plant by PCR. This technique may be used to isolate additional coding sequences from a desired plant or as a diagnostic assay to determine the presence of coding sequences in a plant. Hybridization techniques include hybridization screening of plated DNA libraries (either plaques or colonies; see, for example, Sambrook et al. (1989) Molecular Cloning: A Laboratory Manual (2d ed., Cold Spring Harbor Laboratory Press, Plainview, N.Y.).

Hybridization of such sequences may be carried out under stringent conditions. “Stringent conditions” or “stringent hybridization conditions” refer to conditions under which a probe will hybridize to its target sequence to a detectably greater degree than to other sequences (e.g., at least 2-fold over background). Stringent conditions are sequence-dependent and will be different in different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing). Generally, a probe is less than about 1000 nucleotides in length, preferably less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30% to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50° to 55° C. Exemplary moderate stringency conditions include hybridization in 40% to 45% formamide, 1.0 M NaCl, 1% SDS at 37° C., and a wash in 0.5× to 1×SSC at 55° to 60° C. Exemplary high stringency conditions include hybridization in 50% formamide, 1 M NaCl, 1% SDS at 37° C., and a wash in 0.1×SSC at 60° to 65° C. Optionally, wash buffers may comprise about 0.1% to about 1% SDS. Duration of hybridization is generally less than about 24 hours, usually about 4 to about 12 hours.

Specificity is typically the function of post-hybridization washes, the critical factors being the ionic strength and temperature of the final wash solution. For DNA-DNA hybrids, the Tₘ can be approximated from the equation of Meinkoth & Wahl (1984) Anal. Biochem. 138:267-284: Tₘ=81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization, and/or wash conditions can be adjusted to hybridize to sequences of the desired identity. For example, if sequences with >90% identity are sought, the Tₘ can be decreased 10° C. Generally, stringent conditions are selected to be about 5° C. lower than the Tₘ for the specific sequence and its complement at a defined ionic strength and pH. However, severely stringent conditions can include a hybridization and/or wash at 1, 2, 3, or 4° C. lower than the Tₘ; moderately stringent conditions can include a hybridization and/or wash at 6, 7, 8, 9, or 10° C. lower than the Tₘ; low stringency conditions can include a hybridization and/or wash at 11, 12, 13, 14, 15, or 20° C. lower than the Tₘ. Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution), it is preferred to increase the SSC concentration so that a higher temperature can be used. An extensive guide to the hybridization of nucleic acids is found in Tijssen (1993) Laboratory Techniques in Biochemistry and Molecular Biology—Hybridization with Nucleic Acid Probes, Part I, Chapter 2 (Elsevier, New York); and Ausubel et al., eds. (1995) Current Protocols in Molecular Biology, Chapter 2 (Greene Publishing and Wiley-Interscience, New York). See Sambrook et al. (1989) Molecular Cloning: A Laboratory Manual (2d ed., Cold Spring Harbor Laboratory Press, Plainview, N.Y.).

Also included within the definition of ERECTA-like proteins useful in the present invention are amino acid sequence variants of ERECTA, delta-ERECTA, ERL1, and ERL2 (SEQ ID NOs: 2, 4, 6, or 8, respectively). By “variants” is intended substantially identical sequences. For nucleotide sequences, conservative variants include those sequences that, because of the degeneracy of the genetic code, encode the amino acid sequence of one of the ERECTA-like proteins useful in the methods of the invention. Naturally-occurring allelic variants such as these can be identified with the use of well-known molecular biology techniques, for example, with polymerase chain reaction (PCR) and hybridization techniques as outlined below. Variant nucleotide sequences also include synthetically derived nucleotide sequences, such as those generated, for example, by using site-directed mutagenesis, but which still encode a ERECTA-like protein of the invention. Generally, variants of a particular nucleotide sequence of the invention will have at least about 40%, 50%, 60%, 65%, 70%, generally at least about 75%, 80%, 85%, preferably at least about 90%, 91%, 92%, 93%, 94%, 95%, 96%, 97%, and more preferably at least about 98%, 99% or more sequence identity to that particular nucleotide sequence as determined by sequence alignment programs described elsewhere herein using default parameters.

By “variant” protein is intended a protein derived from the native protein by deletion (so-called truncation) or addition of one or more amino acids to the N-terminal and/or C-terminal end of the native protein; deletion or addition of one or more amino acids at one or more sites in the native protein; or substitution of one or more amino acids at one or more sites in the native protein. Variant proteins encompassed by the present invention are biologically active, that is they continue to possess the desired biological activity of the native protein, as described herein. Such variants may result from, for example, genetic polymorphism or from human manipulation. Biologically active variants of ERECTA-like proteins of the invention will have at least about 40%, 50%, 60%, 65%, 70%, generally at least about 75%, 80%, 85%, preferably at least about 90%, 91%, 92%, 93%, 94%, 95%, 96%, 97%, and more preferably at least about 98%, 99% or more sequence identity to the amino acid sequence of the ERECTA-like protein as determined by sequence alignment programs described elsewhere herein using default parameters. A biologically active variant of a protein of the invention may differ from that protein by as few as 1-15 amino acid residues, as few as 1-10, such as 6-10, as few as 5, as few as 4, 3, 2, or even 1 amino acid residue.

The proteins used in the methods of the invention may be altered in various ways including amino acid substitutions, deletions, truncations, and insertions. Methods for such manipulations are generally known in the art. For example, amino acid sequence variants of the ERECTA-like proteins can be prepared by mutations in the DNA. Methods for mutagenesis and nucleotide sequence alterations are well known in the art (see, for example, Kunkel (1985) Proc. Natl. Acad. Sci. USA. 82:488-492; Kunkel et al. (1987) Methods in Enzymol. 154:367-382; U.S. Pat. No. 4,873,192; Walker & Gaastra, eds. (1983) Techniques in Molecular Biology (MacMillan Publishing Company, New York) and the references cited therein. Guidance as to appropriate amino acid substitutions that do not affect biological activity of the protein of interest may be found in the model of Dayhoff et al. (1978) Atlas of Protein Sequence and Structure (Natl. Biomed. Res. Found., Washington, D.C.), herein incorporated by reference. Conservative substitutions, such as exchanging one amino acid with another having similar properties, may be preferable.

Thus, the genes and nucleotide sequences coding for ERECTA-like proteins include both the naturally-occurring sequences as well as mutant forms. Likewise, ERECTA-like proteins encompass both naturally-occurring proteins as well as variations and modified forms thereof. Such variants will continue to possess the desired activity of modulating plant height and organ shape. Obviously, the mutations that will be made in the DNA encoding the variant must not place the sequence out of reading frame and preferably will not create complementary regions that could produce secondary mRNA structure (see EP Patent Application Publication No. 75,444).

The deletions, insertions, and substitutions of the protein sequences encompassed herein are not expected to produce radical changes in the characteristics of the protein. However, when it is difficult to predict the exact effect of the substitution, deletion, or insertion in advance of doing so, one skilled in the art will appreciate that the effect will be evaluated by routine screening assays, such as described in EXAMPLES 1 and 4. Plants exhibiting modulated plant height or organ shape can be selected using visual observation.

Variant nucleotide sequences and proteins also encompass sequences and proteins derived from a mutagenic and recombinogenic procedure such as DNA shuffling. With such a procedure, one or more different ERECTA-like coding sequences can be manipulated to create a new ERECTA-like sequence possessing the desired properties. In this manner, libraries of recombinant polynucleotides are generated from a population of related sequence polynucleotides comprising sequence regions that have substantial sequence identity and can be homologously recombined in vitro or in vivo. For example, using this approach, sequence motifs encoding a domain of interest may be shuffled between, for example, ERECTA and other known sequences coding for a receptor-like kinase protein to obtain a new gene coding for a protein with an improved property of interest, such as an increased Kₘ in the case of an enzyme. Strategies for such DNA shuffling are known in the art (see, for example, Stemmer (1994) Proc. Natl. Acad. Sci. U.S.A. 91:10747-10751; Stemmer (1994) Nature 370:389-391; Crameri et al. (1997) Nature Biotechnol. 15:436-438; Moore et al. (1997) J. Mol. Biol. 272:336-347; Zhang et al. (1997) Proc. Natl. Acad. Sci. U.S.A. 94:4504-4509; Crameri et al. (1998) Nature 391:288-291; and U.S. Pat. Nos. 5,605,793 and 5,837,458.

The ERECTA-like proteins used in the methods of the invention lack an active kinase domain and modulate plant height and organ shape when expressed in transgenic plants, as described in EXAMPLES 1, 3, and 4. As used herein, an “ERECTA-like protein lacking an active kinase domain” refers to any ERECTA-like protein that no longer possess kinase activity. The kinase domains of ERECTA-like proteins are typically located in the C-terminal cytoplasmic region of the protein. The kinase domains of receptor-like protein kinases in plants are readily identified by the presence of highly conserved residues, protein kinase subdomains, and invariant amino acids (see, e.g., Torii et al. (1996) Plant Cell 8:735-46; (Torii (2000) Curr. Opin. Plant Biol. 3:361-7). Methods for assessing the kinase activity of an ERECTA-like protein are standard in the art.

As discussed above, transgenic expression of ERECTA-like proteins lacking an active kinase domain interfere with the ERECTA signaling pathway resulting, for example, in the production of dwarf plants. Thus, the ERECTA-like proteins used in the methods of the invention have dominant-negative activity. An ERECTA-like family protein lacking an active kinase domain may lack all of the kinase domain or may have a mutation in the kinase domain that destroys the kinase activity of the ERECTA-like protein. A fragment of an ERECTA-like nucleotide sequence that encodes an ERECTA-like protein having dominant-negative activity will encode at least 15, 25, 30, 50, 60, 70, 80, 90, or 94 contiguous amino acids, or up to the total number of amino acids present in a full-length ERECTA-like protein of the invention (for example, 976 amino acids for SEQ ID NO:2). Alternatively, a variant of an ERECTA-like protein of the invention that has dominant-negative activity will have at least about 40%, 50%, 60%, 65%, 70%, generally at least about 75%, 80%, 85%, preferably at least about 90%, 91%, 92%, 93%, 94%, 95%, 96%, 97%, and more preferably at least about 98%, 99% or more sequence identity to the amino acid sequence for the native protein as determined by sequence alignment programs described elsewhere herein using default parameters. In addition, a variant of a ERECTA-like protein having dominant-negative activity may differ from that protein by as few as 1-15 amino acid residues, as few as 1-10, such as 6-10, as few as 5, as few as 4, 3, 2, or even 1 amino acid residue.

In some embodiments of the methods of the invention, the transgene expressing the ERECTA-like protein lacking an active kinase domain encodes an ERECTA-family receptor-like kinase, such as an ERECTA protein, an ERL1 protein, an ERL2 protein, an ERa protein, an ERb protein, or an ERc protein. In some embodiments of the methods of the invention, the transgene expressing the ERECTA-like protein lacking an active kinase domain encodes a protein having an amino acid sequence that is identical to at least one of the sequences provided in SEQ ID NOs:4, 10, 12, and 86-88. In some embodiments, the transgene expressing the ERECTA-like protein lacking an active kinase domain encodes a protein comprising an amino acid sequence that is identical to amino acids 1 to 947, such as amino acids 1 to 650 or 1 to 600 of the sequence provided in SEQ ID NO:2. An exemplary transgene encoding amino acids 1 to 614 of the sequence provided in SEQ ID NO:2 is set forth in SEQ ID NO:3. In some embodiments, the transgene expressing the ERECTA-like protein lacking an active kinase domain encodes a protein comprising an amino acid sequence that is identical to amino acids 1 to 947, such as amino acids 1 to 650 or 1 to 600 of the sequence provided in SEQ ID NO:6. An exemplary transgene encoding amino acids 1 to 612 of the sequence provided in SEQ ID NO:6 is set forth in SEQ ID NO:9. In some embodiments, the transgene expressing the ERECTA-like protein lacking an active kinase domain encodes a protein comprising an amino acid sequence that is identical to amino acids 1 to 950, such as amino acids 1 to 648 or 1 to 600 of the sequence provided in SEQ ID NO:8. An exemplary transgene encoding amino acids 1 to 613 of the sequence provided in SEQ ID NO:8 is set forth in SEQ ID NO:11. In some embodiments, the transgene expressing the ERECTA-like protein lacking an active kinase domain encodes a protein comprising an_amino acid sequence that is identical to at least one of the sequences provided in SEQ ID NOs:86-88.

In some embodiments of the methods of the invention, the transgene expressing the ERECTA-like protein lacking an active kinase domain encodes a protein comprising an amino acid sequence that is at least about 60% identical (such as at least 70%, at least 80%, at least 85%, at least 90%, at least 95%, or at least 99%) to at least one of the sequences provided in SEQ ID NOs:2, 4, 6, 8, 10, 12, 86, 87, and 88.

In some embodiments of the methods of the invention, the ERECTA-like protein lacking an active kinase domain is encoded by a nucleic acid molecule comprises an nucleic acid sequence that is at least about 70% identical (such as at least 80%, at least 85%, at least 90%, at least 95%, or at least 99%) to at least one of the sequences provided in SEQ ID NOs:1, 3, 5, 7, 9, and 11.

In some embodiments of the methods of the invention, the ERECTA-like protein lacking an active kinase domain is encoded by a nucleic acid molecule that hybridizes under stringent conditions to at least one of the sequences provided in SEQ ID NOs:1, 3, 5, 7, 9, and 11.

The term “modulating plant height and organ shape” refers to altering the height of a plant and/or altering the shape of a plant organ. In some embodiments, the methods of the invention reduce the height of the plant. The methods of the invention may also provide strength in the stem. er mutants have a compact, upright growth stature with thicker stems. This prevents inflorescence stems from falling down, getting entangled, and/or losing seeds. Functional assays to identify ERECTA-like proteins that modulate plant height and organ shape include expression of the ERECTA-like fragment or variant in a plant and visually assaying for a modulation in plant height or organ shape, as described in EXAMPLES 1 and 4.

In the methods of the invention, a transgene encoding an ERECTA-like protein lacking an active kinase domain is expressed in a plant. Accordingly, the sequences coding for ERECTA-like proteins used in the methods of the invention are provided in expression vectors for expression in the plant of interest. The vectors generally include 5′ and 3′ regulatory sequences operably linked to a coding sequence for an ERECTA-like protein. The term “operably linked” refers to a functional linkage between a promoter and a second sequence, wherein the promoter sequence initiates and mediates transcription of the DNA sequence corresponding to the second sequence. Generally, operably linked means that the nucleic acid sequences being linked are contiguous and, where necessary to join two protein coding regions, contiguous and in the same reading frame. The expression vector may additionally contain selectable marker genes.

The expression vector generally includes, in the 5′-3′ direction of transcription, a transcriptional and translational initiation region, a sequence coding for an ERECTA-like protein, and a transcriptional and translational termination region functional in plants. The transcriptional initiation region, the promoter, may be native (or analogous) or foreign (or heterologous) to the plant host. Additionally, the promoter may be the natural sequence or alternatively a synthetic sequence. By “foreign” or “heterologous” is intended that the transcriptional initiation region is not found in the native plant into which the transcriptional initiation region is introduced.

The termination region may be native with the transcriptional initiation region, may be native with the operably linked DNA sequence of interest, or may be derived from another source. Convenient termination regions are available from the Ti-plasmid of A. tumefaciens, such as the octopine synthase and nopaline synthase termination regions (see also Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; Joshi et al. (1987) Nucleic Acid Res. 15:9627-9639).

Where appropriate, the sequences coding for an ERECTA-like protein may be optimized for increased expression in the transformed plant. That is, the genes can be synthesized using plant-preferred codons for improved expression. Methods are available in the art for synthesizing plant-preferred genes (see, for example, U.S. Pat. Nos. 5,380,831, and 5,436,391; Murray et al. (1989) Nucleic Acids Res. 17:477-498, herein incorporated by reference).

Additional sequence modifications are known to enhance gene expression in a cellular host. These include elimination of sequences encoding spurious polyadenylation signals, exon-intron splice site signals, transposon-like repeats, and other such well-characterized sequences that may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures.

The expression vector may additionally contain 5′ leader sequences. Such leader sequences can act to enhance translation. Translation leaders are known in the art and include: picomavirus leaders, for example, EMCV leader (Encephalomyocarditis 5′ noncoding region) (Elroy-Stein et al. (1989) Proc. Natl. Acad. Sci. USA. 86:6126-6130); polyvirus leaders, for example, TEV leader (Tobacco Etch Virus) and MDMV leader (Maize Dwarf Mosaic Virus) and human immunoglobulin heavy-chain binding protein (BiP), (Macejak et al. (1991) Nature 353:90-94); untranslated leader from the coat protein mRNA of alfalfa mosaic virus (AMV RNA 4) (Jobling et al. (1987) Nature 325:622-625); tobacco mosaic virus leader (TMV) (Gallie et al. (1989) in Molecular Biology of RNA, ed. Cech (Liss, New York), pp. 237-256); and maize chlorotic mottle virus leader (MCMV) (Lommel et al. (1991) Virology 81:382-385. Other methods known to enhance translation can also be utilized, for example, introns, and the like.

Generally, the expression vector comprises a selectable marker gene for the selection of transformed cells. Selectable marker genes include genes encoding antibiotic resistance, such as those encoding neomycin phosphotransferase II (NEO) and hygromycin phosphotransferase (HPT), as well as genes conferring resistance to herbicidal compounds, such as glufosinate ammonium, bromoxynil, imidazolinones, and 2,4-dichlorophenoxyacetate (2,4-D). Any selectable marker gene can be used in the present invention.

A number of promoters may be used in the practice of the invention, such as tissue-specific, temporally specific, inducible, or ubiquitous promoters. The promoters may be selected based on the desired outcome. Constitutive promoters include, for example, the core promoter of the Rsyn7 (PCT Application Serial No. US99/03863); Scp1 promoter (U.S. Pat. No. 6,072,050), rice actin (McElroy et al. (1990) Plant Cell 2:163-171; Zhang et al. (1991) Plant Cell 3:1155-65); ubiquitin (Christensen et al. (1989) Plant Mol. Biol. 12:619-632; Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730); ALS promoter (U.S. application Ser. No. 08/409,297), 35S, and the like. Other constitutive promoters are described, for example, in U.S. Pat. Nos. 5,608,149; 5,608,144; 5,604,121; 5,569,597; 5,466,785; 5,399,680; 5,268,463; and 5,608,142.

In some embodiments it may be beneficial to express the gene from an inducible promoter. For example, chemical-regulated promoters can be used to modulate the expression of a gene in a plant through the application of an exogenous chemical regulator. Depending upon the objective, the promoter may be a chemical-inducible promoter, where application of the chemical induces gene expression, or a chemical-repressible promoter, where application of the chemical represses gene expression. Chemical-inducible promoters are known in the art and include, but are not limited to, the maize In2-2 promoter, which is activated by benzenesulfonamide herbicide safeners, the maize GST promoter, which is activated by hydrophobic electrophilic compounds that are used as pre-emergent herbicides, and the tobacco PR-1a promoter, which is activated by salicylic acid. Other chemical-regulated promoters of interest include steroid-responsive promoters (see, for example, the glucocorticoid-inducible promoter in Schena et al. (1991) Proc. Natl. Acad. Sci. USA. 88:10421-10425; McNellis et al. (1998) Plant J. 14(2):247-257) and tetracycline-inducible and tetracycline-repressible promoters (see, for example, Gatz et al. (1991) Mol. Gen. Genet. 227:229-237; U.S. Pat. Nos. 5,814,618 and 5,789,156), herein incorporated by reference. Other inducible promoters include drought-inducible promoters, which refers to a promoter that is inducible under conditions of osmotic stress (see for example, Vilardell et al. (1990) Plant Mol. Biol. 14:423-432; Urao et al. (1993) Plant Cell 5:1529-1539); Guerrero et al. (1988) Plant Physiol. 88:401-408; Guerrero et al. (1990) Plant Mol. Biol. 15: 11-26; Guerrero et al. (1993) Plant Mol. Biol. 21:929-935; U.S. Pat. No. 6,084,153; Uno et al. (2000) Proc. Natl. Acad. Sci. U.S.A. 97:11632-11637; Yamaguchi-Shinozaki et al. (1993) Mol. Gen. Genet. 236:331-40; Yamaguchi-Shinozaki et al. (1994) Plant Cell 6:251-64, all of which are herein incorporated by reference).

Alternatively, tissue-specific promoters may be utilized to target enhanced expression of ERECTA-like proteins within a particular plant tissue. Tissue-specific promoters include, for example, shoot meristem-preferred promoters (see, for example, Atanassova et al. (1992) Plant J. 2:291; U.S. Pat. No. 6,239,329; which is herein incorporated by reference). In addition, the promoter of the KNOTTED1 gene can be used to direct shoot meristem-specific expression (Dorien et al. (2002) Plant Mol. Biol. 48:423-441; Tomoaki et al. (2001) Genes Dev. 15:581-590, which are herein incorporated by reference). Alternatively, the promoter of the REVOLUTA gene could be used for meristem-specific expression (see, for example, Genbank Accession No. AC024594 (Rice) and AB005246 (Arabidopsis), both of which are herein incorporated by reference.

In some embodiments of the invention, the promoter used comprises 5′ regulatory sequences and/or 3′ regulatory sequences from the ERECTA locus, as described in EXAMPLES 1 and 4. Exemplary ERECTA 5′ regulatory sequences are provided in SEQ ID NO:13. Exemplary ERECTA 3′ regulatory sequences are provided in SEQ ID NO:14. In some embodiments of the invention, the promoter used comprises the 35S promoter and/or the 35S dual terminator from CaMV, as described in EXAMPLE 3. Exemplary CaMV 35S promoter and 35S dual terminator sequences are provided in SEQ ID NOs:15 and 16, respectively.

According to the methods of the invention, the expression vector for expressing the ERECTA-like protein is introduced into a plant. The methods of the invention do not depend on a particular method for introducing the expression vector into a plant, as long as the expression vector gains access to the interior of at least one cell of the plant. Methods for introducing expression vectors into plants are known in the art including, but not limited to, stable transformation methods, transient transformation methods, and virus-mediated methods.

The term “stable transformation” refers to introducing an expression vector into a plant such that it integrates into the genome of the plant and is capable of being inherited by progeny thereof. The term “transient transformation” refers to introducing an expression vector into a plant such that it does not integrate into the genome of the plant.

The expression vectors of the invention may be introduced into plants by contacting plants with a virus or viral nucleic acids. Generally, such methods involve incorporating a nucleotide construct of the invention within a viral DNA or RNA molecule. It is recognized that the a ERECTA-like protein of the invention may be initially synthesized as part of a viral polyprotein, which later may be processed by proteolysis to produce the desired recombinant protein. Further, it is recognized that promoters of the invention also encompass promoters utilized for transcription by viral RNA polymerases. Methods for introducing expression vectors into plants and expressing a protein encoded therein, involving viral DNA or RNA molecules, are known in the art (see, for example, U.S. Pat. Nos. 5,889,191; 5,889,190; 5,866,785; 5,589,367 and 5,316,931; herein incorporated by reference).

The method of transformation/transfection is not critical to the instant invention; various methods of transformation or transfection are currently available. Thus, any method, which provides for effective transformation/transfection may be employed. Transformation protocols as well as protocols for introducing nucleotide sequences into plants may vary depending on the type of plant or plant cell, i.e., monocot or dicot, targeted for transformation. Suitable methods of introducing nucleotide sequences into plant cells and subsequent insertion into the plant genome include microinjection (Crossway et al. (1986) Biotechniques 4:320-334), electroporation (Riggs et al. (1986) Proc. Natl. Acad. Sci. U.S.A. 83:5602-5606, Agrobacterium-mediated transformation (U.S. Pat. No. 5,563,055; U.S. Pat. No. 5,981,840), direct gene transfer (Paszkowski et al. (1984) EMBO J. 3:2717-2722), and ballistic particle acceleration (see, for example, U.S. Pat. No. 4,945,050; Tomes et al. (1995) in Plant Cell, Tissue, and Organ Culture: Fundamental Methods, ed. Gamborg and Phillips (Springer-Verlag, Berlin); McCabe et al. (1988) Biotechnology 6:923-926; Weissinger et al. (1988) Ann. Rev. Genet. 22:421-477; Sanford et al. (1987) Particulate Sci. Technol. 5:27-37 (onion); Christou et al. (1988) Plant Physiol. 87:671-674 (soybean); McCabe et al. (1988) Bio/Technology 6:923-926 (soybean); Finer & McMullen (1991) In Vitro Cell Dev. Biol. 27P:175-182 (soybean); Singh et al. (1998) Theor. Appl. Genet. 96:319-324 (soybean); Datta et al. (1990) Biotechnology 8:736-740 (rice); Klein et al. (1988) Proc. Natl. Acad. Sci. U.S.A. 85:4305-4309 (maize); Klein et al. (1988) Biotechnology 6:559-563 (maize); U.S. Pat. Nos. 5,240,855, 5,322,783, and 5,324,646; Tomes et al. (1995) in Plant Cell, Tissue, and Organ Culture: Fundamental Methods, ed. Gamborg (Springer-Verlag, Berlin) (maize); Klein et al. (1988) Plant Physiol. 91:440-444 (maize); Fromm et al. (1990) Biotechnology 8:833-839 (maize); Hooykaas-Van Slogteren et al. (1984) Nature 311:763-764; Bytebier et al. (1987) Proc. Natl. Acad. Sci. U.S.A. 84:5345-5349 (Liliaceae); De Wet et al. (1985) in The Experimental Manipulation of Ovule Tissues, ed. Chapman et al. (Longman, N.Y.), pp. 197-209 (pollen); Kaeppler et al. (1990) Plant Cell Reports 9:415-418; Kaeppler et al. (1992) Theor. Appl. Genet. 84:560-566 (whisker-mediated transformation); D'Halluin et al. (1992) Plant Cell 4:1495-1505 (electroporation); Li et al. (1993) Plant Cell Reports 12:250-255 and Christou and Ford (1995) Ann. Botany 75:407-413 (rice); Osjoda et al. (1996) Nature Biotechnol. 14:745-750 (maize via Agrobacterium tumefaciens); all of which are herein incorporated by reference).

The cells that have been transformed may be grown into plants in accordance with conventional methods (see, for example, McCormick et al. (1986) Plant Cell Reports 5:81-84). These plants may then be grown, and either pollinated with the same transformed strain or different strains, and the resulting hybrid having constitutive expression of the desired phenotypic characteristic identified. Two or more generations may be grown to ensure that constitutive expression of the desired phenotypic characteristic is stably maintained and inherited and then seeds harvested to ensure constitutive expression of the desired phenotypic characteristic has been achieved.

The methods of the invention may be used for making transgenic plants of any plant species, including, but not limited to, monocots and dicots. Examples of plants of interest include, but are not limited to, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassaya (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers.

Vegetables of interest include, but are not limited to, tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo).

Ornamentals of interest include, but are not limited to, azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum. Conifers that may be employed in practicing the present invention include, for example, pines such as loblolly pine (Pinus taeda), slash pine (Pinus elliotii), ponderosa pine (Pinus ponderosa), lodgepole pine (Pinus contorta), and Monterey pine (Pinus radiata); Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis).

In another aspect, the invention provides methods for enhancing the yield of a crop plant, comprising the steps of: (a) introducing a transgene into a crop plant, wherein the transgene encodes an ERECTA-like protein lacking an active kinase domain and wherein expression of the transgene enhances the yield of the crop plant; and (b) growing the transgenic crop plant under conditions in which the transgene is expressed to enhance the yield of the crop plant. As described above, dwarf plants may be created by expressing an ERECTA-like protein lacking an active kinase domain in a plant. The term “enhancing the yield of a crop plant” refers to increasing the harvest of grain or seed per plant compared to a regular-size plant of the same species, and/or to increasing the harvest of grain or seed per unit area of arable land. An advantage of dwarf crop plants is that they direct less resources (for example, nutrients and energy) into making vegetative tissue and more resources into making seeds or grain compared to normal-size plants, thereby increasing the yield of crop per plant. In addition, more dwarf plants may be planted per unit area of arable land, thereby further increasing the yield of crop. In some embodiments, the crop plant is rice or canola.

A further aspect of the invention provides transgenic plants comprising a gene encoding an ERECTA-like protein lacking an active kinase domain. In some embodiments, the transgenic plant is selected from the list of plants of interest provided above.

Yet another aspect of the invention provides vectors comprising a nucleic acid sequence encoding an ERECTA-like protein lacking an active kinase domain and host cells and/or cell cultures (e.g., plant cell cultures) comprising these vectors. The invention also provides nucleic acid molecules comprising the sequence of SEQ ID NO:5 or SEQ ID NO:7, or sequences substantially identical thereto.

The following examples merely illustrate the best mode now contemplated for practicing the invention, but should not be construed to limit the invention.

### EXAMPLE 1

This Example describes an exemplary method of the invention for modulating the growth or form of a plant by expressing a truncated form of the receptor kinase ERECTA in transgenic Arabidopsis plants.

Loss-of-function erecta mutations confer a compact inflorescence with short internodes and clustered flower buds, short pedicels, round flowers, and short, blunt siliques (Bowman (1993) Arabidopsis: An Atlas of Morphology and Development (Springer-Verlag, New York); Torii et al. (1996) Plant Cell 8:735-46). Despite these defects, the erecta mutation does not affect organ identity, polarity, or tissue organization. As such, Landsberg erecta has been used widely as a “wild type” because of its preferable, compact plant size. Cellular defects caused by erecta are not documented extensively; however, both cell size and number are altered in erecta inflorescence stems (Komeda et al. (1998) J. Plant Res. 111:701-13). Consistent with its role in organogenesis, ERECTA is expressed at high levels in the entire shoot apical meristem and developing organs (Yokoyama et al. (1998) Plant J. 15:301-10). ERECTA encodes a Leu-rich repeat receptor-like kinase (LRR-RLK) with functional Ser/Thr kinase activity (Torii et al. (1996) Plant Cell 8:735-46; Lease et al. (2001) New Phytol. 151:133-44). The LRR-RLKs constitute the largest subfamily of plant RLKs and possess a structural organization similar to that of animal receptor kinases (Torii (2000) Curr. Opin. Plant Biol. 3:361-7; Shiu & Bleecker (2001) Proc. Natl. Acad. Sci. U.S.A. 98:10763-8). Several LRR-RLKs function as important developmental regulators, including Arabidopsis CLAVATA1 (CLV1), which balances cell proliferation and differentiation in the meristem, RLK5/HAESA, which promotes flower abscission, and the brassinosteroid (BR) receptor BRI1 (Clark et al. (1997) Cell 89:575-85; Li & Chory (1997) Cell 90:929-38; Jinn et al., 2000). The mutant phenotypes, expression patterns, and molecular identity of ERECTA as an LRR-RLK support the notion that ERECTA mediates yet to be identified cell-to-cell signaling pathways that coordinate shoot organ growth.

Expression of truncated receptor kinases has been used widely as a powerful tool to reveal in vivo function and signal transduction of animal receptor kinases. For both animal receptor Tyr kinases (RTK) and transforming growth factor-beta receptor Ser/Thr kinases, the general consensus is that truncated receptors that lack the cytoplasmic kinase domain act as dominant-negative receptors by blocking the normal activity of the endogenous counterparts (Amaya et al. (1991) Cell 66:257-70; Ueno et al. (1991) Science 252:844-7; Hemmati-Brivanlou & Melton (1992) Nature 359:609-14; Freeman (1996) Cell 87:651-60). Such an approach has not been pursued actively in plant RLK studies because of frequent cosuppression events (Conner et al. (1997) Plant J. 11:809-23; Schumacher & Chory (2000) Curr. Op. Plant Biol. 3:79-84) or the inability to detect the accumulation of the transgene products (He et al. (1998) Plant J. 14:55-63). An additional confusion in understanding the modes of action of the plant RLK is that the kinase domain of LRR-RLK appears dispensable, because truncated mutations that remove the entire kinase domain of two LRR-RLKs, CLV1 and rice Xa21, retain partial activity (Clark et al. (1997) Cell 89:575-85; Wang et al. (1998) Plant Cell 10:765-79; Torii (2000) Curr. Opin. Plant Biol. 3:361-7). This Example shows that truncated ERECTA protein that lacks the cytoplasmic kinase domain (delta-Kinase, SEQ ID NO:4) interferes with endogenous ERECTA function. Therefore, unlike CLV1 and Xa21, delta-Kinase of ERECTA acts as a dominant-negative receptor. Importantly, the delta-Kinase protein enhances the phenotype of the null erecta plants. delta-Kinase migrates as an about 400-kD protein complex in the absence of the endogenous ERECTA protein, suggesting that delta-Kinase associates with other RLKs and/or ligands, that are shared by other RLKs, and blocks their functions. Based on cell biological analysis of the erecta mutants and delta-Kinase transgenic plants, it is likely that multiple overlapping and interrelated RLK signaling pathways, including ERECTA, are required for coordinated cell proliferation and cell growth within the same tissue types during Arabidopsis organogenesis.

Methods

Plant Materials and Growth Conditions: Arabidopsis thaliana ecotype Columbia was used as the wild type. erecta-103 and erecta-105 were backcrossed four times into the wild type before use (Torii et al. (1996) Plant Cell 8:735-46). Plants were grown on soil mixture (Sunshine Mix4:vermiculite:perlite, 2:1:1, Sun Gro Horticulture Canada, Seba Beach, Canada) supplemented with 0.85 mg/cm³ Osmocoat 14-14-14 (Scotts, Maysville, Ohio) under an 18-h-light/6-h-dark cycle at 21 C.

Generation of Transgenic Plants Expressing delta-Kinase: To construct the plasmid carrying the truncated ERECTA, a stop codon was introduced by PCR behind the putative transmembrane domain at amino acid position 615. PCR was performed using pKUT196, which contains the entire ERECTA locus from Columbia, as a template and primers Erg5858link (5′ ATGAATTCTGTCTGCAGTGTCAATCTCTA 3′, SEQ ID NO:17) and ER6000Bam.rc (5′ TCAGGATCCTATGATCCATCAAGAAAAGGAGG 3′, SEQ ID NO:18). The amplified fragment was digested with PstI and BamHI and introduced into plasmid pKUT197 to generate pESH101. The EcoRI-BamHI fragment of pESH101, which contains the 1.7-kb ERECTA promoter and the coding region of the truncated ERECTA, was cloned into pKUT531, a pPZP222-based binary vector, which contains the 1.9-kb ERECTA terminator (Hajdukiewicz et al. (1994) Plant Mol. Biol. 25:989-94). The plasmid was named pESH201. To generate the delta-Kinase-c-Myc construct, the following cloning steps were performed. To introduce the BamHI site after Ser-615 of ERECTA, PCR was performed using pKUT196 as a template and primers Erg5868link (5′ ATGAATTCTGTCTGCAGTGTCAATCTCTA 3′, SEQ ID NO:17) and ER-6000link.rc (5′ TCAGGATCCGCTGATCCATCAAGAAAAGGAGG 3′, SEQ ID NO:19). The amplified fragment was cloned into PstI-BamHI-digested pKUT197 to generate pESH113. The triple c-Myc sequence was amplified by PCR using primers myc-5 (5′ GAAGATCTCGAGTTCGGTGAACAAAAGTT 3′, SEQ ID NO:20) and myc-3 (5′ CGGGATCCTTACCCTAGCTTTCCGTTCAAGT 3′, SEQ ID NO:21) with pSLJ13471 as a template (Jones et al. (1992) Transgenic Res. 1:285-97). The amplified fragment was digested with BglII and BamHI and inserted into BamHI-digested pESH113. The resulting plasmid, pESH115, contains the additional sequence 5′ ADLEFG(EQKLISEEDLNG)₃KLG 3′ (SEQ ID NO:22) after Ser-615 of ERECTA. EcoRI-BamHI-digested pESH115 was cloned into pKUT531 to generate pESH215. To generate delta-KinaseM282I, PCR was performed using erecta-103 genomic DNA as a template with primers ERg1761 (5′ GTATATCTAAAAACGCAGTCG 3′, SEQ ID NO:23) and ERg2339rc (5′ CAACAACATTGAAGGTGACATTTT 3′, SEQ ID NO:24). The amplified fragment was digested with SpeI and SacI and replaced the SpeI-SacI fragment of pESH101. The resulting plasmid, pKUT572.3, was digested with AflII and SacI and inserted into pESH201 to generate pKUT574.3. The sequences of all fragments created by PCR were confirmed. pESH201, pESH215, and pKUT574.3 were introduced into Agrobacterium tumefaciens strain GV3101/pMP90 by electroporation and into Arabidopsis wild-type and erecta-105 plants using the vacuum infiltration method (Bechtold et al. (1993) Acad. Sci. Paris 916:1194-9).

Scanning Electron Microscopy: Tissue samples were fixed overnight in 4% (v/v) glutaraldehyde in 25 mM NaPO₄ buffer, pH 7.0, and subsequently with 1% osmium tetroxide in 25 mM NaPO₄ buffer for 4 to 5 days at 4° C. The samples were dehydrated with a graded series of ethanol, critical point dried, sputter coated with gold, and observed with a scanning electron microscope (JEOL 840A).

Light Microscopy: Tissue samples were fixed overnight in 4% (v/v) paraformaldehyde in 25 mM NaPO₄ buffer, pH 7.0, at 4° C., dehydrated with a graded series of ethanol, and infiltrated with polymethacryl resin Technovit 7100 (Heraeus Kulzer, Wehrheim, Germany) followed by embedding and polymerization in Technovit 7100. Nine-micrometer sections were prepared using a Leica RM-6145 microtome (Wetzlar, Germany). The tissue sections were stained with 0.1% toluidine blue in 0.1 M NaPO₄ buffer, pH 7.0, and observed under bright-field illumination.

Antiserum Production and Purification: The cDNA sequence encoding the extracellular domain of ERECTA (ERLRR; amino acids 36 to 577) was amplified using primers ERLRRab5 (5′ CGGAATTCTCATTCAAAGATGTGAACAATG 3′, SEQ ID NO:25) and ERLRRab3 (5′ CGTCTAGACTATGACACTCGTACAGTTCGA 3′, SEQ ID NO:26), with pKUT161 as a template (Torii et al. (1996) Plant Cell 8:735-46). The amplified fragment was inserted in the EcoRI-XbaI-digested modified pSP73_AatII vector (Promega) to generate pKUT534. The sequence was confirmed. Subsequently, the fragment was inserted in pMal-c2 vector (New England Biolabs, Beverly, Mass.) and pGEX4T-1 vector (Amersham Pharmacia Biotech) to generate pKUT535 (maltose binding protein [MBP]-ERLRR) and pKUT538 (glutathione S-transferase [GST]-ERLRR), respectively. The fusion proteins were expressed in Escherichia coli BL21/DE3(pLysS). The inclusion bodies of E. coli expressing MBP-ERLRR were separated by SDS-PAGE, and the recombinant protein was excised from the gel. Polyclonal ERLRR antisera were raised in rabbits at Cocalico Biologicals (Reamstown, Pa.). Affinity purification of antibodies was performed using the GST-ERLRR fusion protein immobilized on nitrocellulose membranes.

Protein Gel Blot and Immunoblot Analyses: One gram of Arabidopsis bud clusters (inflorescence tips) was ground in liquid nitrogen, mixed with 2 mL of ice-cold lysis buffer (50 mM Tris, pH 7.5, 1 mM EDTA, 100 mM NaCl, 0.1% SDS, 0.1% Triton X-100, 0.7% beta-mercaptoethanol, and 1 mM phenylmethylsulfonyl fluoride) and 0.5 mL of loading buffer (200 mM Tris-HCl, pH 6.8, 8% SDS, 0.4% bromphenol blue, 40% glycerol, and 10% beta-mercaptoethanol), and boiled for 5 minutes. Proteins were separated by 8% SDS-PAGE. SeeBlue prestained protein standards (Invitrogen, Carlsbad, Calif.) were used as molecular mass markers. For visualization of the total proteins, the gel was stained with Coomassie Brilliant Blue R250. For immunoblot analysis, the proteins were transferred from the gel to Hybond enhanced chemiluminescence nitrocellulose membranes (Amersham Pharmacia Biotech) using a semidry blotting apparatus (Owl Separation Systems, Portsmouth, N.H.). The membranes were blocked with 5% BSA in PBS for 2 hours at room temperature and probed with primary antibody at a dilution of 1:15 for the affinity-purified ERLRR polyclonal antibody or 1:600 for the 9E10 anti-c-Myc monoclonal antibody (Covance, Richmond, Calif.) in PBS with 1% BSA at 4° C. overnight. Goat anti-rabbit and sheep anti-mouse horseradish peroxidase-linked antibodies were used as secondary anti-ERLRR and anti-c-Myc antibodies, respectively, at a dilution of 1:35,000 in 0.1% Tween 20/PBS for 1 hour at room temperature. The detection of ERECTA, delta-Kinase, and delta-Kinase-c-Myc was performed with the Chemiluminescence assay kit (Amersham Pharmacia Biotech).

Reverse Transcriptase-Mediated PCR: Total RNA was isolated from Arabidopsis bud clusters using the RNeasy Plant Mini Kit (Qiagen, Valencia, Calif.) and treated with DNaseI Amp grade (Gibco BRL). First-strand cDNA was synthesized from 2 micrograms of RNA with random hexamer primers using the ThermoScript reverse transcriptase-mediated PCR system (Gibco BRL) according to the manufacturer's instructions. PCR was performed with 0.5 microliters of the first-strand reaction at 96° C. for 2 minutes, then with varying numbers of cycles at 96° C. for 35 seconds, 60° C. for 45 seconds, 72° C. for 90 seconds, and then at 72° C. for 10 minutes. The primers ERK7 (5′ CACAGAGACGTGAAGTCGT 3′, SEQ ID NO:27) and ERg7361rc (5′ AGCTTAACGCAACGAAAAGATACC 3′, SEQ ID NO:28) were used to amplify endogenous ERECTA. The primers ERg5022 (5′ CTTGAGTAGAAATCATATAACT 3′, SEQ ID NO:29) and ERg5757rc (5′ TGACACGGTGAGTTTAGCCAA 3′, SEQ ID NO:30) were used to simultaneously amplify both the endogenous ERECTA and the introduced delta-Kinase. The primers ERg5022 (5′ CTTGAGTAGAAATCATATAACT 3′, SEQ ID NO:31) and ERg7361.rc (5′ AGCTTAACGCAACGAAAAGATACC 3′, SEQ ID NO:28) were used to amplify the introduced delta-kinase. Transcripts of the actin gene were amplified as a control using primers ACT2-1 (5′ GCCATCCAAGCTGTTCTCTC 3′, SEQ ID NO:32) and ACT2-2 (5′ GCTCGTAGTCAACAGCAACAA 3′, SEQ ID NO:33). WUS transcripts were amplified as described by Hamada et al. (2000). Reverse transcriptase-mediated PCR products were electrophoresed on agarose gels and visualized by staining with ethidium bromide.

Far-Western Analysis of the ERECTA-KAPP Interaction: For protein-protein interaction (far-western) blot analysis, the kinase domains of ERECTA and RLK5 were expressed as MBP fusions. The ERECTA kinase domain (corresponding to amino acids 611 to 977) was amplified from cDNA using the primers ERK4 (5′ CGGAATTCACTAGTACCATGGACAAACCAGTAACTTATTCG 3′, SEQ ID NO:34) and ER3rc (5′ CGGGATCCACTAGTGCATAATACTTTACATGAGA 3′, SEQ ID NO:35). The amplified fragment was digested with EcoRI and BamHI and introduced into pSP73-delta-AatII to generate pKUT503. To make a kinase-inactive version of ERECTA, the invariant Lys (Lys-676) was replaced with a Glu. The first round of PCR was performed with the primer pairs ERK4 and ERK13/K676E (5′ CTGTGGGTTGTGAGAGTAAAGCCGTTCAATCGCAACCG 3′, SEQ ID NO:36) and ERKI5/K676E (5′ TTGAACGGCTTTACTCTCACAACCCA 3′, SEQ ID NO:37) and ERCodeC3 (5′ CGGGATCCACTAGTCTACTCACTGTTCTGAGAAATAACTT 3′, SEQ ID NO:38). In the next round, products from both reactions were mixed and amplified with ERK4 and ERCodeC3. The amplified fragment was digested with EcoRI and BamHI and introduced into the plasmid pSP73-delta-AatII to generate pKUT536. The EcoRI-SalI fragments of both pKUT503 and pKUT536 were cloned into pMAL-c2 (New England Biolabs). The plasmids RLK5CAT-MBP, RLK5CAT(K711E)MBP, and GST-KID 134 were generous gifts from John Walker (University of Missouri, Columbia). The recombinant MBP and GST fusion proteins were expressed in E. coli strain AD494/DE3 and purified by affinity chromatography on amylose-agarose resin (New England Biolabs) or glutathione-Sepharose 4B resin (Amersham Pharmacia Biotech), respectively, according to the manufacturers' instructions. GST-KID was labeled with ³²P as described (Braun et al. (1997) Plant J. 12:83-95). Protein concentrations were determined with Bio-Rad Protein Assay Solution. One milligram each of ERCAT-MBP, ERCAT(K676E)-MBP, RLK5CAT-MBP, and RLK5CAT(K711E)-MBP was blotted onto a Hybond enhanced chemiluminescence nitrocellulose membrane (Amersham Pharmacia Biotech), and far-western analysis with radiolabeled KID protein was performed as previously described (Braun et al. (1997) Plant J. 12:83-95).

Gel Filtration Analysis: The Arabidopsis bud clusters were ground in liquid nitrogen, mixed with 2 volumes of ice-cold extraction buffer (50 mM Hepes, pH 7.4, 10 mM EDTA, 1% Triton X-100, and 1% protease inhibitor cocktail [Sigma]), filtered through Miracloth (Calbiochem), and centrifuged at 1500 g for 10 minutes at 4° C. The supernatant was ultracentrifuged subsequently at 100,000 g for 1 hour at 4° C. Membrane proteins from ERECTA::delta-Kinase-c-Myc/erecta-105 bud clusters were isolated in a similar manner except that no Triton X-100 was added to the extraction buffer and pellet instead of supernatant was recovered. Subsequently, the pellet was resuspended in extraction buffer containing 1% Triton X-100, and the supernatant was used for chromatography. Gel filtration was performed using a fast protein liquid chromatography system (Amersham Pharmacia Biotech) with a Superose 6 HR10/30 column (Amersham Pharmacia Biotech) at a flow rate of 0.4 ml/minute. Column equilibration and chromatography were performed in the following buffer: 0.05 mM NaPO₄, pH 7.3, 0.05 mM NaCl, 0.02% Na azide, and 1% Triton X-100. Next, 0.2-ml fractions were collected and concentrated by incubation with trichloroacetic acid (20% final concentration) for 30 minutes on ice and subsequent centrifugation at 13,500 rpm for 10 minutes at 4° C. The precipitates were washed with acetone, vacuum-dried, and resuspended in 20 microliters of loading buffer. Concentrated fractions were subjected to immunoblotting and probed with either anti-c-Myc or anti-ERECTA LRR antibodies as described above. High and low molecular mass gel-filtration calibration kits (Amersham Pharmacia Biotech) were used as molecular mass standards.

Flow Cytometry: For flow cytometric analysis of Arabidopsis nuclear DNA content, 50 to 100 mg of mature pedicel tissues was collected from 4- to 6-week-old plants. To ensure the uniformity of the samples, pedicels bearing flower buds, flowers, youngest five siliques, and siliques turning yellow were discarded. The pedicel samples were chopped finely in 1.5 ml of the ice-cold extraction buffer (15 mM Hepes, 1 mM EDTA, 80 mM KCl, 20 mM NaCl, 300 mM sucrose, 0.2% Triton X-100, 0.5 mM spermine, and 0.1% beta-mercaptoethanol) for 3 minutes, passed through the filter, and centrifuged at 13,000 rpm for 1 minute. The pellet was resuspended with 650 ml of the staining buffer (0.1 mg/mL propidium iodide and 100 microgram/ml RNase A in the extraction buffer), passed through the filter, and subjected to analysis using FACSI (Becton-Dickinson, Franklin Lakes, N.J.) at the Cell Analysis Facility (Department of Immunology, University of Washington). For each measurement, 50,000 events were recorded at 560 V of the FL2 channel. At least two independent extractions were performed for each genotype, and two or three independent measurements were performed for each extraction.

Results

Transgenic Plants That Express delta-Kinase Display the erecta Mutant Phenotype: A truncated ERECTA that retains the extracellular LRR and transmembrane domains but lacks the cytoplasmic kinase domain (delta-Kinase) was introduced into Arabidopsis wild-type ERECTA plants (ecotype Columbia). To ensure the proper temporal/spatial expression patterns of the truncated ERECTA, the 1.7-kb 5′ and 1.9-kb 3′ regions of the Columbia ERECTA locus, which correspond to the ERECTA promoter and terminator, respectively (Yokoyama (1998) Plant J. 15:301-10), as well as a genomic fragment of delta-Kinase, which contains all 23 introns (Torii et al. (1996) Plant Cell 8:735-46), were used to express delta-Kinase. The delta-Kinase fragment contains a short cytoplasmic tail of 12 amino acids, which is juxtaposed to the putative transmembrane domain. Fifty-one of 54 independent T1 plants showed a phenotype resembling that of loss-of-function erecta mutant plants. Analysis of the selfed T2 progeny revealed that the phenotype was dominant and linked tightly to the transgene. Among the lines that contained a single T-DNA insertion, two lines (L1 and L2) with strong phenotype and one line (L3) with mild phenotype were chosen for a further characterization. Transgenic ERECTA::delta-Kinase plants had short stature and developed a compact inflorescence, short pedicels, and short, blunt siliques, all of which are reminiscent of loss-of-function erecta mutant plants. The ERECTA::delta-Kinase inflorescence tip displayed a characteristic clustering, which was indistinguishable from that of the intermediate allele erecta-103 (Torii et al. (1996) Plant Cell 8:735-46). Detailed morphometric analysis revealed that plant height and pedicel length of ERECTA::delta-Kinase plants were intermediate between those of the wild type and the null allele erecta-105, with L3 being tallest, as shown in Table 1, which documents the results of a morphogenetic analysis of fully grown 7-week-old plants of wild type, erecta-105, and three independent transgenic lines (L1, L2, and L3) of ERECTA::delta-Kinase. Twenty-five plants were analyzed for each plant (inflorescence) height. Lengths of 50 mature pedicels and siliques on the main inflorescence stem (10 measurements per stem) were analyzed. Silique lengths of all three lines were as short as that of erecta-105 (Table 1). The morphology of the silique tips was analyzed in detail. The tip of the wild-type silique had an elongated style that protruded from narrow valves. By contrast, the tips of the erecta and ERECTA: delta-Kinase siliques (L2) had short and broad styles.

TABLE 1
Morphogenetic Analysis of ERECTA::delta-Kinase Plants

| | Plant Height | Pedicle Length | Silique Length |
|---|---|---|---|
| Genotype | (cm +/− SD) | (mm +/− SD) | (mm +/− SD) |
| wild type | 47.0 +/− 4.4 | 9.1 +/− 1.1 | 14.7 +/− 0.9 |
| erecta-105 | 24.5 +/− 4.0 | 4.1 +/− 0.6 | 11.2 +/− 0.9 |
| ERECTA::delta- | 31.3 +/− 4.3 | 4.9 +/− 0.7 | 11.0 +/− 0.9 |
| Kinase L1 | | | |
| ERECTA::delta- | 30.0 +/− 2.6 | 5.4 +/− 0.5 | 11.2 +/− 1.0 |
| Kinase L2 | | | |
| ERECTA::delta- | 35.7 +/− 6.4 | 7.5 +/− 0.6 | 11.1 +/− 1.2 |
| Kinase L3 | | | |

These results suggest that the organ elongation defects conferred by ERECTA::delta-Kinase highly resemble the disruption of normal ERECTA function. The transgenic plants also displayed reduced fertility as a result of defective elongation of the stamen filaments.

Accumulation of the delta-Kinase Fragment Confers Dominant-Negative Interference: The erecta phenotype conferred by the introduction of ERECTA::delta-Kinase could be attributable to dominant-negative interference of the ERECTA pathway by the truncated ERECTA receptor. Alternatively, it could be the result of the cosuppression of endogenous ERECTA gene expression. To distinguish between these two possibilities, the level of the endogenous ERECTA transcripts in three transgenic lines was examined. Reverse transcriptase-mediated (RT) PCR analysis using primers that anneal to the kinase domain revealed that levels of the endogenous ERECTA transcripts were not altered significantly by the transgene, excluding the possibility of cosuppression.

From immunoblots probed with antibody raised against the ERECTA LRR domain (anti-ERLRR), endogenous ERECTA was detected as a band of about 145 kD in both wild-type and transgenic plants. In the erecta-105 null allele background, a very faint band was detected at a similar position, likely representing LRR-RLKs closely related to ERECTA. The higher molecular mass of ERECTA compared with its calculated molecular mass (105 kD) suggests a possible glycosylation, because the extracellular domain of ERECTA possesses 12 potential N-glycosylation sites (Torii et al. (1996) Plant Cell 8:735-46). Several other plant LRR receptors, including tomato Cf-4/Cf-9 and the carrot phytosulfokine receptor, have been shown to be glycosylated (Piedras et al. (2000) Plant J. 21:529-36; Matsubayashi et al. (2002) Science 296:1470-2; Rivas et al. (2002) Plant J. 29:783-96; Rivas et al. (2002) Plant Cell 14:689-702). The delta-Kinase protein migrates at about 95 kD (the predicted polypeptide is 64 kD) and therefore may be glycosylated as well.

Interestingly, it was found that the delta-Kinase protein was accumulated at much higher levels than the full-length, endogenous ERECTA protein. A quantitative analysis of the immunoblot signals estimated that the amount of delta-Kinase was about 100 times greater in L1 and L2 and about 30 times greater in L3 than the endogenous ERECTA. RT-PCR analysis with primers that amplify both endogenous and truncated ERECTA revealed that the amounts of delta-Kinase transcripts were at levels comparable to those of the endogenous ERECTA transcripts. Therefore, the increased amount of delta-Kinase was associated with post-transcriptional regulation, most likely as a result of the increased stability of the truncated protein.

Both RT-PCR with primers specific to the delta-Kinase transgene and immunoblot analysis demonstrated that ERECTA::delta-Kinase was expressed at a level three times greater in the lines with severe phenotype (L1 and L2) than in the line with mild phenotype (L3). Thus, the phenotypic severity correlates with the amount of delta-Kinase gene products in a dosage-dependent manner.

From these results, it is most likely that the observed erecta phenotype of ERECTA::delta-Kinase transgenic plants is conferred by dominant-negative interference of highly stable delta-Kinase protein with the endogenous ERECTA pathway. Thus, the apparent discrepancy in the recessive nature of erecta mutations and the dominant effects of ERECTA::delta-Kinase can be explained by the high level of accumulation of ΔKinase protein. Although mRNA levels of delta-Kinase and endogenous ERECTA are similar, the amount of the delta-Kinase protein is about 100 fold higher than the full-length ERECTA protein, most likely due to increased protein stability. In animals, ligand-induced degradation of the EGF (epidermal growth factor) RTK plays an important role in down-regulation of EGF signaling (Beguinot et al. (1984) Proc. Natl. Acad. Sci. U.S.A. 81:2384-8; Jones et al. (2002) Am. J. Physiol. Cell. Physiol. 282:C420-33). Similarly, the amount of endogenous ERECTA RLK may be tightly regulated during organogenesis. Perhaps the truncated delta-Kinase is no longer under such regulation, and thus it stably locks in and sequesters the signaling components.

A delta-Kinase Fragment Further Enhances the Growth Defects in the Null Allele of erecta: The dominant-negative effects of delta-Kinase were quite surprising, given that similar deletion mutants in CLV1 and Xa21 (e.g., clv1-6 and Xa21-D) have been shown to retain partial function (Clark et al. (1997) Cell 89:575-85; Wang et al. (1998) Plant Cell 10:765-79; Torii (2000) Curr. Opin. Plant Biol. 3:361-7). Therefore, attempts were made to determine the underlying mechanism of the dominant-negative interference. If the ERECTA signaling pathway is strictly homodimeric and linear, it is predicted that the expression of delta-Kinase will not enhance the erecta null phenotype. By contrast, delta-Kinase may make the erecta null phenotype even more severe if the ERECTA signaling pathway is redundant. To address these hypotheses, ERECTA::delta-Kinase was introduced into erecta-105 plants, which do not produce any ERECTA transcripts (Torii et al. (1996) Plant Cell 8:735-46; Lease et al. (2001) Plant Cell 13:2631-41). Expression of delta-Kinase in erecta-105 conferred severe growth defects, as shown in Table 2, which presents the results of a morphometric analysis of fully grown 7-week-old plants of erecta-105, three independent transgenic lines of ERECTA: delta-Kinase/erecta-105 (L1, L2, and L3), and one line of ERECTA::delta-Kinase-c-Myc/erecta-105. Plant (inflorescence) height, pedicel length, and silique length were measured as described above. The length of the siliques was measured in only one line of ERECTA::delta-Kinase/erecta-105 (L3) because the other two lines had reduced fertility as a result of short filaments.

TABLE 2
Morphogenetic Analysis of ERECTA::delta-Kinase/erecta-105
Plants

| | Plant Height | Pedicle Length | Silique Length |
|---|---|---|---|
| Genotype | (cm +/− SD) | (mm +/− SD) | (mm +/− SD) |
| erecta-105 | 24.5 +/− 4.0 | 4.1 +/− 0.6 | 11.2 +/− 0.9 |
| ERECTA::delta- | 19.6 +/− 1.1 | 1.7 +/− 0.5 | 7.3 +/− 1.1 |
| Kinase-c- | | | |
| Myc/erecta-105 | | | |
| ERECTA::delta- | 15.8 +/− 1.5 | 1.9 +/− 0.3 | N/A |
| Kinase/erecta-105 | | | |
| L1 | | | |
| ERECTA::delta- | 16.2 +/− 1.6 | 1.7 +/− 0.3 | N/A |
| Kinase/erecta-105 | | | |
| L2 | | | |
| ERECTA::delta- | 16.4 +/− 1.3 | 1.9 +/− 0.5 | 4.6 +/− 0.6 |
| Kinase/erecta-105 | | | |
| L3 | | | |

The transgenic plants were dwarf, with extremely short internodes, pedicels, and siliques as well as smaller, round flowers. The development of flower organs seemed less coordinated, and pistils protruded above buds. ERECTA::delta-Kinase-c-Myc, which contains a triple c-Myc sequence at the end, retained the ability to exaggerate the erecta null phenotype, albeit slightly less effectively. This finding could be attributable to steric hindrance by the triple c-Myc peptides or, alternatively, to reduced accumulation of the gene products. Immunoblot analysis revealed that delta-Kinase-c-Myc was detected by both anti-ERLRR and anti-c-Myc antibodies as a band of about 105 kD, slightly larger than the nontagged delta-Kinase. The anti-c-Myc antibody was highly specific and essentially gave no background signal.

Although both delta-Kinase and delta-Kinase-c-Myc confer phenotypes much more severe than that of erecta-105 or any of the available 24 erecta alleles (Lease et al. (2001) New Phytol. 151:133-44), the phenotypic characteristics, such as compact inflorescence and short, blunt siliques, were consistent with the erecta defects. Together, these data support the hypothesis that nonfunctional delta-Kinase is capable of interfering with and shutting down the ERECTA and related RLK pathways that regulate organ elongation in a partially redundant manner.

The Highly Accumulated ERECTA delta-Kinase Fragment Does Not Interfere with the CLV1 LRR-RLK Signaling Pathway: Use of the endogenous ERECTA promoter and terminator should minimize the ectopic effects of delta-Kinase. However, it is possible that a highly stable delta-Kinase fragment associates in a nonspecific manner with RLKs that are expressed in the same tissue/cell types as ERECTA but that do not normally interact with the ERECTA signaling pathway. Experiments were conducted to determine whether the dominant-negative delta-Kinase inhibits a well-studied RLK signaling pathway that has overlapping expression patterns with ERECTA.

For this purpose, it was investigated whether ERECTA::delta-Kinase inhibits the CLV1 signaling pathway. CLV1 is expressed at the subepidermal layers in the center of the shoot and flower meristems, whereas ERECTA is expressed broadly within these meristems (Clark et al. (1997) Cell 89:575-85; Yokoyama (1998) Plant J. 15:301-10); therefore, delta-Kinase likely accumulates in the cells in which CLV1 normally functions. The hallmark of the clv phenotype is an increased number of floral organs as a result of enlarged floral meristems (Leyser & Furner (1992) Development 116:397-403; Clark et al. (1993) Development 119:397-418). Although the wildtype flower has two carpels, average carpel numbers of the severe allele clv1-4 and the weak allele clv1-6 are 5.12+/−0.04 and 3.91+/−0.04, respectively (Yu et al. (2000) Development 127:1661-70). Carpel numbers were not affected by the expression of ERECTA::delta-Kinase. Wild type, erecta-105, delta-Kinase in wild-type, and delta-Kinase in erecta-105 all produced siliques with two carpels (2.00+/−0.00, n=40 for each genotype).

Molecular-genetic studies have shown that the CLV signaling pathway restricts the expression domain of WUSCHEL (WUS), which specifies stem cell fate (Brand et al. (2000) Science 289:617-9; Schoof et al. (2000) Cell 100:635-44). Unlike clv mutations, which confer ectopic upregulation of WUS expression (Brand et al. (2000) Science 289:617-9; Schoof et al. (2000) Cell 100:635-44), semiquantitative RT-PCR analysis revealed that ERECTA::delta-Kinase had no effect on WUS expression levels. These phenotypic and molecular analyses imply that highly accumulated delta-Kinase does not interfere with the CLV signaling pathway.

These findings suggest that the components of ERECTA and CLV signaling pathways are quite distinct, even though the structural features of these two LRR-RLKs are similar. Therefore, it was investigated whether ERECTA associates with a known component of the CLV pathway, Kinase-Associated Protein Phosphatase (KAPP). KAPP associates with the kinase domains of several RLKs, including RLK5/HAESA and CLV1, via its kinase interaction domain (KID) (Stone et al. (1994) Science 266:793-5; Stone et al. (1998) Plant Physiol. 117:1217-24; Williams et al. (1997) Proc. Natl. Acad. Sci. U.S.A. 94:10467-72). Both wild-type and kinase-inactive forms of ERECTA fused to the maltose binding protein were expressed. The kinase-inactive version (K676E) has a substitution of an invariant Lys in subdomain II to Glu. The interaction of ERECTA with the KAPP KID was tested by dot-blot analysis, because it gave stronger signals than electroblotted samples. Positive signal was detected only in the kinase-active form of RLK5/HAESA, which was used as a positive control. Because ERECTA possesses Ser/Thr protein kinase activity (Lease et al. (2001) New Phytol. 151:133-44), it is concluded that ERECTA does not associate with KAPP in vitro. These results indicate that the expression of the delta-Kinase fragment of ERECTA does not affect the CLV1 pathway, which most likely operates in a distinct manner; they further imply that the dominant-negative effects of delta-Kinase involve specific mechanisms.

Although highly-accumulated delta-Kinase protein may also interfere with factors that do not normally interact with the endogenous ERECTA, it is reasonable to conclude that the observed dominant-negative interference is highly specific for the following reasons. First, the growth defects conferred by delta-Kinase resemble or exaggerate the phenotypes of the erecta mutations, both in overall plant morphology and underlying cellular defects. Second, the ERECTA cis regulatory sequences we used to express delta-Kinase contain information sufficient for a proper expression of ERECTA, since a full-length ERECTA clone with these regulatory sequences fully complements erecta mutants). Therefore, neomorphic effects of delta-Kinase in different tissue/cell types should be minimized. Third, delta-Kinase does not inhibit the CLV LRR-RK signaling pathway, which operates in the same cells that express ERECTA within the shoot and flower meristems. Fourth, introduction of a point mutation in the LRR domain of delta-Kinase abolished the dominant negative effects without affecting stability of the transcripts/proteins (see below). This suggests that proper interaction with ligands and/or receptor partners that normally associate with native ERECTA are likely required for the observed dominant-negative interference, rather than the about 100 fold accumulation of the protein causing some cellular toxicity.

That having truncated ERECTA protein is worse than having none at all for Arabidopsis organ and internodal growth suggests complex redundancy in the signaling pathways involving ERECTA. One possible model is that several RLKs are capable of perceiving the same signal as ERECTA and regulate partially overlapping pathways. The delta-Kinase may take up and deplete ligands for other receptors and/or may directly interact with multiple receptor partners of ERECTA, and thus shut down whole pathways, which could operate either in parallel or convergent manners. The fact that a functional LRR domain is required for dominant-negative interference (see below) supports this hypothesis.

Such intricacy in signal transduction is well known in numerous animal RKs. For example, mammalian PDGF (platelet-derived growth factor) exists as homodimers, as well as heterodimers of three homologous polypeptides: PDGF A, B, and C (such as AA, AB, and BB) (Ataliotis & Mercola (1997) Int. Rev. Cytol. 172:95-125; Li et al. (2000) Nat. Cell Biol. 2:302-9). Two PDGF RTKs, PDGFαR and PDGFβR, recognize different PDGF isoforms with distinct affinity (Ataliotis & Mercola (1997) Int. Rev. Cytol. 172:95-125). This complex ligand-receptor recognition property provides overlapping yet unique functions for PDGF signaling during mammalian embryogenesis. Consistently, expression of the dominant-negative delta-Kinase form of PDGFR suppressed diverse signal transduction pathways mediated by multiple PDGF isoforms (Ueno et al. (1993) J. Biol. Chem. 268:22814-9). Similar complexity is documented for FGF (fibroblast growth factor) RTK signaling pathways (Givol & Yayon (1992) FASEB J. 6:3362-9).

A Functional LRR Domain of ERECTA Is Required for the Dominant-Negative Effects: To gain more insight into the mechanisms of delta-Kinase action, a point mutation corresponding to erecta-103, which replaces the Met within the 10th LRR with Ile (M282I) (Torii et al. (1996) Plant Cell 8:735-46), was introduced into the delta-Kinase fragment. Introduction of the mutation did not result in reduced stability of the transcripts or proteins; instead, the amount of the delta-KinaseM282I protein appeared to have increased slightly. Nevertheless, the M2821 mutation severely compromised the dominant-negative effects of delta-Kinase, because transgenic erecta-105 plants expressing delta-KinaseM282I no longer displayed severe dwarfism, extreme compact inflorescence, or reduced fertility. These results indicate that a functional LRR domain is required for the dominant-negative interference and imply that structural integrity of the extracellular LRR domain may be crucial for titrating ligand or receptor partners that are shared by RLKs, which possess overlapping function with ERECTA.

Dominant-Negative delta-Kinase Migrates as a Protein Complex in the Absence of Endogenous ERECTA: If delta-Kinase confers dominant-negative interference through direct association with the components (such as ligands and partners) of related RLKs, delta-Kinase would be expected to form a protein complex. To test this hypothesis, the behavior of delta-Kinase was investigated by gel-filtration chromatography. Flowers and bud clusters of transgenic erecta-105 plants expressing either delta-Kinase or delta-Kinase-c-Myc were used as materials to minimize the complications of having both full-length and truncated ERECTA. In the presence of 1% Triton X-100, the delta-Kinase protein migrated as a complex of about 400 kD. No signal of similar size was detected in the control erecta-105 fractions. Some delta-Kinase may exist at about 100 kD, representing monomers. The presence of nonspecific signals of similar size in the control erecta-105 fractions, however, makes this possibility inconclusive. The immunoblot probed with anti-c-Myc antibodies revealed that delta-Kinase-c-Myc migrated exclusively as a complex of a similar size with a slightly broader range of elution, which may be the result of less efficient interactions of delta-Kinase-c-Myc with other components caused by steric hindrance. The fact that delta-Kinase migrated as a protein complex in the absence of the endogenous ERECTA is consistent with the hypothesis that the physical interaction of nonfunctional delta-Kinase with the other RLKs enhances the organ elongation defects in erecta-105.

As described above, delta-Kinase migrates as an about 400 kDa protein complex in the absence of endogenous ERECTA protein. This complex most likely represents a non-functional receptor oligomer, suggesting that ERECTA may function as a hetero-oligomer. Some plant LRR-RLKs function as heterodimers. For example, CLV 1 forms a heterodimer CLV2 LRR-transmembrane protein, presumably via disulfide linkage (Jeong et al. (1999) Plant Cell 11:1925-33; Trotochaud et al. (1999) Plant Cell 11:393-405). Recently, the Arabidopsis BAK1 LRR-RLK was identified as a receptor partner of BRI1 in BR signaling (Li et al. (2002) Cell 110:213-22; Nam & Li (2002) Cell 110:203-12).

The formation of an about 400 kDa delta-Kinase protein complex is consistent with a recent view that plant LRR receptors constitute membrane-associated complexes (Trotochaud et al. (1999) Plant Cell 11:393-405; Rivas et al. (2002) Plant J. 29:783-96; Rivas et al. (2002) Plant Cell 14:689-702). However, the components of the receptor complex could be distinct among CLV1, Cfs, and ERECTA. For instance, while active CLV1 complex contains KAPP and ROP small GTPase, neither is in the Cf4- and Cf9 complex (Trotochaud et al. (1999) Plant Cell 11:393-405; Rivas et al. (2002) Plant J. 29:783-96; Rivas et al. (2002) Plant Cell 14:689-702). It was found that ERECTA does not associate with KAPP. Since the delta-Kinase complex is non-functional, it is also unlikely to contain cytoplasmic factors that are recruited to the complex in a phosphorylation dependent manner, such as ROP (Trotochaud et al. (1999) Plant Cell 11:393-405). On the other hand, by analogy to the animal dominant-negative RKs, the delta-Kinase complex likely contains ligands and receptor partners for ERECTA and related RLKs. This is consistent with the finding that the point mutation within the LRR domain disrupts dominant-negative interference.

ERECTA Regulates Proper Cell Proliferation and Polarity: To understand how ERECTA controls organ elongation, we analyzed the cellular defects in erecta and in dominant-negative transgenic plants were analyzed. Mature pedicels were examined, because the degree of allelic severity correlates with reduction in pedicel length (Torii et al. (1996) Plant Cell 8:735-46; Lease et al. (2001) New Phytol. 151:133-44). Although the wild-type pedicels were approximately twice as long as erecta-105 pedicels, cells in the cortex and endodermis of erecta-105 were notably larger than wild-type cells, indicating that the short pedicel phenotype is attributable to fewer cells. Moreover, cortex cells were expanded radially, accounting for the thick pedicel phenotype of erecta. The epidermal cells of erecta-105 were slightly shorter than the wild-type cells. No significant difference in the pith were detected. These observations indicate that erecta is not a typical dwarf mutant with general cell elongation defects.

Similar to the erecta mutation, the delta-Kinase protein conferred reduced cell numbers associated with enlarged and irregular cell shape in the cortex and endodermis. However, unlike erecta-105, delta-Kinase cortex cells were not expanded laterally. In the ERECTA::delta-Kinase/erecta-105 pedicels, disorganized cell growth in the cortex was even more evident. Although some cells in the cortex were large and expanded, others remained small, leaving many “gaps” between the cells. These observations suggest that ERECTA and its overlapping pathways are required for coordinated cell proliferation and proper cell-cell interactions within the cortex cell layers.

The observations that erecta confers greatly increased cell size, primarily in the cortex, despite the fact that overall organ elongation is strongly inhibited is in contrast to almost all known dwarf mutants, which have reduced cell size, including those defective in biosynthesis and/or perception of hormones, such as. auxins, gibberellins, and BRs (Timpte et al. (1992) Planta 188:271-8; Szekeres et al. (1996) Cell 85:171-92; Azpiroz et al. (1998) Plant Cell 10:219-30; Fridborg et al. (1999) Plant Cell 11:1019-32), and indicate that ERECTA does not promote the general cell elongation process. The cellular defects in erecta are rather similar to the inhibition in cell cycle progression, which leads to cell enlargement although overall plant size is reduced (Wang et al. (2000) Plant J. 24:613-23; De Veylder et al. (2001) Plant Cell 13:1653-68).

Increased Levels of Somatic Endoploidy in Pedicels of erecta and delta-Kinase Plants: Increased cell size in general correlates with increased DNA content or ploidy level (Kondorosi et al. (2000) Curr. Op. Plant Biol. 3:488-92). Because mature pedicels of erecta and delta-Kinase have enlarged cortex cells, their ploidy levels were measure to determine whether the inhibition of ERECTA signaling leads to somatic endoploidy. A majority (62%) of nuclei in the wild-type pedicels remained diploid (2C), whereas some were tetraploid (4C; 31%) and a few were octaploid (8C; 7%). Both intermediate allele erecta-103 and delta-Kinase pedicels showed increases in the 4C nuclei (37 and 36%, respectively), making the 2C/4C ratio 1.5, in contrast to 2.0 in the wild-type pedicels. The 4C nuclei content was highest in the null allele erecta-105 pedicels (49%; 2C/4C ratio=0.9), whose cortex cells were the largest. Therefore, the degree of erecta defects and cortex cell size have a positive correlation with increased 4C content. The expression of delta-Kinase in erecta-105 did not confer an additional increase in 4C content (47%; 2C/4C ratio=0.97). This finding is consistent with the histological observation that delta-Kinase/erecta-105 did not lead to extra cell enlargement but rather disrupted the proper coordination of cortex cell development. None of the genotypes showed increased amounts of 8C, indicating that inhibition of the ERECTA pathway does not activate endoreduplication cycles. Because mature pedicels do not express the cell-cycling marker Cyc1At::GUS, it is very unlikely that the 4C nuclei represent actively proliferating S-phase cells. Together, these findings suggest that erecta mutations and delta-Kinase expression may inhibit cell division and promote premature differentiation of the 4C cells.

Consistent with the hypothesis that ERECTA is required for proper cell cycle progression, the ratio of 4C cells increased both in erecta and delta-Kinase plants. Perhaps in the absence of the ERECTA signal, the cortex cells in pedicels may not enter mitosis and instead undergo premature differentiation at the G2 stage. In contrast to erecta, overexpression of the cyclin kinase inhibitors (ICKs/KRPs) in Arabidopsis reduced the ratio of 4C cells in the leaf due to slowed cell cycle progression and reduced endoreduplication (De Veylder et al. (2001) Plant Cell 13:1653-68). Therefore, ERECTA signaling and ICKs/KRPs may act on a distinct aspect of cell proliferation.

The striking cellular phenotype of the delta-Kinase/erecta-105 pedicels is a loss of the organized cortex cell size and shape, suggesting that a loss of the entire pathway not only inhibits cell proliferation but also disrupts the uniformity of cell proliferation. Perhaps ERECTA and overlapping signaling pathways provide positional cues for coordinated proliferation among cells of the same type and such coordination is essential for proper organ elongation.

### EXAMPLE 2

This Example describes the identification and use of two ERECTA-family receptor-like kinases that control organ growth and flower development.

Methods

Plant Materials and Growth Conditions: The Arabidopsis ecotype Columbia (Col) was used as a wild type. T-DNA knockout seed population that contains erl1-2 and erl2-1 mutants was obtained from the Arabidopsis Biological Resource Center. All mutant lines were backcrossed three times to Col wild-type plants prior to any phenotypic analysis. Plants were grown in a condition as previously described (Shpak et al. (2003) Plant Cell 15:1095-1110).

Cloning of ERL1 and ERL2: RT-PCR was performed with wild-type cDNA as a template using primer pairs: (for ERL1) ERL1.14coding (5′ GGCTCTTTCAGCAACTTAGT 3′, SEQ ID NO:39) and ERL1g6054rc (5′ CTTCTGCATCAGGATTCCTAACTT 3′, SEQ ID NO:40); and (for ERL2) ERL2.3coding (5′ GGCGATAAAGGCTTCATTCA 3′, SEQ ID NO:41) and ERL2g5352rc (5′ TTGTATCTGAAGAGTGGCTCTCAC 3′, SEQ ID NO:42). The 5′ ends of mRNA were recovered by a rapid amplification of cDNA ends (RACE) using FirstChoice™ RLM RACE kit (Ambion, Austin, Tex.). Elk1-300rc (5′ TCCATATAACAGATTCTC 3′, SEQ ID NO:43) or Ekl2-300.rc (5′ TCCATATAACAGATTCTC 3′, SEQ ID NO:44) was used as outer primer and Elk1-185rc (5′ CGTAGGTCTCCAATAGCTGGA 3′, SEQ ID NO:45) or Elk2-185rc (5′ ATCAAATCTCCAAGGGCAGAT 3′, SEQ ID NO:46) was used as nested primer for ERL1 and ERL2, respectively. The amplified fragments were cloned into pCR2.1-TOPO (Invitrogen, Carlsbad, Calif.) and sequenced.

Reverse transcriptase-mediated (RT) PCR: RNA isolation, cDNA synthesis, and RT-PCR were performed as previously described (Shpak et al. (2003) Plant Cell 15:1095-1110) with various cycles. Primer pairs used are as follows. ERECTA: ERg4359 (5′ CAACAATGATCTGGAAGG AC 3′, SEQ ID NO:47) and ERg5757rc (5′ TGACACGGTGAGTTTAGCCAA 3′, SEQ ID NO:30); ERL1: ERL1g2846 (5′ TATCCCACCGATACTTGGCA 3′, SEQ ID NO:48) and ERL1g4411rc (5′ CCGGAGAGATTGTTGAAGGA 3′, SEQ ID NO:49); ERL2: ERL2g3085 (5′ CTGTCTGGCAACAATTTCTCA 3′, SEQ ID NO:50) and ERL2g4254rc (5′ -AGCCATGTC CATGTGAAGAA 3′, SEQ ID NO:51); ANT: 5′ant-1 (5′ GCCCAACACGACTACAAA C3′, SEQ ID NO:52) and ANT1600rc (5′ TCATATCTACCAGTCCATCTAT 3′, SEQ ID NO:53); STM: STM781 (5′ TGGAGATCCATCATAACGAAAT 3′, SEQ ID NO:54) and STM2354rc (5′ GACCCATTATTGTTCCTATCAA 3′, SEQ ID NO:55); WUS: U3WUS5 (5′ GTGAACAAAAGTCGAATCAAACACACATG 3′, SEQ ID NO:56) and U34WUS3rc (5′ GCTAGTTCAGACGTAGCTCAAGAG 3′, SEQ ID NO:57); KNAT1: BP681 (5′ GCTCCTCAAGAATCAATC A 3′, SEQ ID NO:58) and BP3100rc (5′ AAGCTATAAGTAGCAAACTGATGTAG 3′, SEQ ID NO:59); CyclinD2: CycD2.501 (5′ ATGGCTGAGAATCTTGCTTG 3′, SEQ ID NO:60) and CycD2.801rc (5′ ATTTAGAATCCAATCAAGAGC 3′, SEQ ID NO:61); CyclinD3: CycD3.501 (5′ TGGATTTAGAAGAGGAGGAA 3′, SEQ ID NO:62) and CycD3.935rc (5′ AAGGAACACGGATCTCTTCAA 3′, SEQ ID NO:63); Actin: ACT2-1 (5′ GCCATCCAAGCTGTTCTCTC 3′, SEQ ID NO:32) and ACT2-2 (5′ GCTCGTAGTCAACAGCAACAA 3′, SEQ ID NO:33).

Complementation of erecta by ERL1 and ERL2: A full-length genomic coding region of ERL1 and ERL2 were cloned into the ERECTA promoter-terminator cassette by the following procedure. PCR was performed with the wild-type Col genomic template using primer pairs: (For ERL1) ERL1 g3036 (5′ GTCACGTCTCAGCTATTTGTAAGCTTGTT 3′, SEQ ID NO:64) and ERL1-3endrc (5′ CGTCTAGATTATATGCTACTTTTGGAGATG 3′, SEQ ID NO:65); and (for ERL2) ERL2g2166 (5′ GCCTATTCCACCAATACTTG 3′, SEQ ID NO:66) and ERL2-3endrc (5′ CGTCTAGATTATAAGCTACTTTTGGAGATA 3′, SEQ ID NO:67). The amplified fragments were digested with SpeI and XbaI and inserted into SpeI-digested pKUT522 to generate pESH208A (for ERL1) and pESH209A (for ERL2). Subsequently, PCR was performed using primer pairs: (for ERL1) ERL1-5end (5′ GCTCTAGAAATGAAGGAGAAGATGCAGC 3′, SEQ ID NO:68) and ERL1g4411rc (5′ CCGGAGAGATTGTTGAAGGA 3′, SEQ ID NO:49); and (for ERL2) ERL2-5end (5′ GCTCTAGAGATGAGAAGGATAGAGACCA 3′, SEQ ID NO:69) and ERL2g3182rc (5′ ACAAATCTGAGAGAGTTAATGCAAAGCAG 3′, SEQ ID NO:70). The amplified fragments were digested with SpeI and XbaI and inserted into SpeI-digested pESH208A and pESH209A respectively, to generate pESH208 (ER::ERL1) and pESH209 (ER::ERL2). The plasmids were introduced into Agrobacterium tumefaciens strain GV3101/pMP90 by electroporation and into Arabidopsis erecta-105 plants by vacuum infiltration.

ERECTA::GUS, ERL1::GUS and ERL2::GUS Transgenic Plants: For construction of ERECTA::GUS, the GUS gene was inserted as an SpeI fragment into pKUT522 between ERECTA promoter and terminator. The plasmid was named pNI101. To make ERL1::GUS and ERL2::GUS constructs, the EcoRI/PstI fragment of pRT2-GUS was cloned into pZP222 (Hajdukiewicz et al. (1994) Plant Mol. Biol. 25:989-94). The plasmid was named pESH244. The ERL1 promoter region was amplified with primers ERL1 g-3680link (5′ AGGAATTCACACCAATAAAAATACACAGCA 3′, SEQ ID NO:71) and ERL1g403linkrc (5′ AGGAATTCGTCGACTTCTTCTTATTCTTCTTTCCTTTTG G 3′, SEQ ID NO:72) using MMI1 BAC clone as a template. The ERL2 promoter region was amplified with primers ERL2g-4364link (5′ AGGAATTCGTGATTAGGAGACGAGGTAGATA 3′, SEQ ID NO:73) and ERL2g4linkrc (5′ AGGAATTCGTCGACCTTCTTCTTCTTCTTCCTCAAGA 3′, SEQ ID NO:74) using T28J14 BAC clone as a template. The amplified fragments were digested with EcoRI and inserted into pESH244. The plasmids were named pESH245 (ERL1::GUS) and pESH246 (ERL2::GUS). pNI101, pESH245 and pESH246 were introduced in Arabidopsis wild type as described above. The GUS histochemical analysis was performed as previously described (Sessions et al. (1999) Plant J. 20:259-63).

Screening and Isolation of the Arabidopsis T-DNA Insertion Mutants: Screening and isolation of T-DNA insertion lines were performed as described by the Arabidopsis KO Facility (http://www.biotech.wisc.edu/Arabidopsis/). The erl1-2 was isolated from α population (vector pD991, kanamycin resistance), and erl2-1 was isolated from β population (vector pROK2, basta resistance) using gene-specific primers and JL-202 T-DNA left border primer (5′ CATTTTATAATAACGCTGCGGACATCTAC 3′ SEQ NO:75). The gene-specific PCR primers were as follows: (For ERL1) ERLK765 (5′ TACCCAATACTTAGCTCTGGGCTTGTTC TT 3′, SEQ ID NO:76) and ERLK6137rc (5′ TCCTTCCAATCAGCATTACTATCTTCCTT 3′, SEQ ID NO:77); (For ERL2) ERTJ70 (5′ AACAACGAAGGTTCTAGCTCTTTCAAAAT 3′, SEQ ID NO:78) and ERTJ5855rc (5′ ACAAGTGAACAACACATCTCCATCAATTA 3′, SEQ ID NO:79). Precise locations of the insertions were determined by sequencing the PCR fragments. Both erl1-2 and erl2-1 were backcrossed three times. The B3F2 populations of erl1-2 and erl2-1 exhibited 3:1 ratio of kanamycin- and Basta resistance, respectively (for erl1-2: KanR:KanS=163:58, χ^{2=0.183}, p=0.669; for erl2-1: BastaR:BastaS=203:76, χ^{2=0.747}, p=0.388), indicating a single T-DNA insertion. The PCR-based genotyping confirmed that these single insertions disrupt the ERL loci.

Generation of Double- and Triple-Knockout Plants: To generate erecta erl1 and erecta erl2 double mutants, erl1-2 and erl2-1 plants were crossed with erecta-105 plants. To generate erl1 erl2 double mutants, erl1-2 plants were crossed with plants of the genotype erecta-105/erecta-105 erl1-2/erl1-2 erl2-1/+. Plants of a correct genotype were isolated from the F2 populations. erecta-105/erecta-105 erl1-21+erl2-1/erl2-1 plants were self-fertilized to obtain the erecta erl1 erl2 triple mutants. The T-DNA insertion that disrupts the ERL1 locus in erl1-2 and ERL2 locus in erl2-1 conferred resistance to kanamycin and Basta, respectively. Thus, progenies of each cross were first tested for the resistance, and subsequently a genotype of individual plants, whether they are heterozygous or homozygous, was determined by PCR using gene-specific primer pairs and a combination of T-DNA- (JL-202) and gene-specific primers. The presence of erecta-105 mutation was determined by PCR using the primer pairs: ERg2248 (5′ AAGAAGTCATCTAAAGATGTGA 3′, SEQ ID NO:80) and er-105 (5′ AGCTGACTATACCCGATACTGA 3′, SEQ ID NO:81) (Torii et al. (2003) in Morphogenesis and Patterning of Biological Systems (ed. T. Sekimura, Tokyo, Japan: Springer-Verlag) pp. 153-64).

Light and Scanning Electron Microscopy: Fixation, embedding, and sectioning of tissues for light microscopy using Olympus BX40, as well as preparation of samples for scanning electron microscopy using JOEL 840A were performed as previously described (Shpak et al. (2003) Plant Cell 15:1095-1110).

Cell number measurement: Light microscopy images of four regions of sectioned wild type, erecta-105, erecta-105 erl1-2 and erecta-105 erl2-1 pedicels were taken and number of cells in a middle longitudinal cortex row was determined. This number was used to calculate the total number of cells in the cortex row of an average length pedicel. Number of cells was counted in three sectioned erecta-105 erl1-2 erl2-1 pedicels and average was determined.

Results

ERL1 and ERL2, two ERECTA-like LRR-RLKs in Arabidopsis: To identify candidate RLKs that act in parallel pathways with ERECTA, the Arabidopsis genome was surveyed and two ERECTA-LIKE genes were found, ERL1 (At5g62230.1) and ERL2 (At5g07180.1). Full-length cDNA clones for ERL1 and ERL2 were subsequently isolated by a combination of RT-PCR and 5′ RACE-PCR. Among 223 Arabidopsis genes encoding LRR-RLKs (Shiu & Bleecker (2003) Plant Physiol. 132:530-43), ERECTA possesses an unusual, characteristic exon-intron structure with 26 introns (Torii et al. (1996) Plant Cell 8:735-46). A comparison of genomic and cDNA sequences reveals that ERL1 and ERL2 also contain 26 introns, all of which are located at identical positions to the introns of ERECTA. The predicted ERL1 and ERL2 proteins share high overall sequence identity to ERECTA (60% identity, 72% similarity) and even higher between each other (78% identity and 83% similarity). The LRR- and the kinase domain possess the highest degree of sequence conservation. The extracellular paired cysteine regions adjacent to the LRR region and the juxtamembrane domain have relatively high sequence identity, while the N-terminal signal sequence and the C-terminal tail region are poorly conserved. The phylogenetic, parsimony analysis suggests that ERL1 and ERL2 have evolved by recent duplication and that they are immediate paralogs of ERECTA. The result is consistent with the neighbor-jointing analysis of the LRR-RLK phylogeny previously reported (Shiu & Bleecker (2001) Proc. Natl. Acad. Sci. USA. 98:10763-8; Yin et al. (2002) Proc. Natl. Acad. Sci. USA. 99:9090-2). The finding that ERL1, ERL2, and ERECTA constitute a subfamily of LRR-RLKs opens the possibility that two ERLs may have functions related to ERECTA.

ERL1 and ERL2 rescue erecta phenotype when expressed under the ERECTA promoter and terminator: To investigate whether ERL1 and ERL2 genes are functional homologs of ERECTA, ERL1 and ERL2 were expressed in the null allele erecta-105 under the control of the native ERECTA promoter and terminator. Both constructs rescued the erecta defects. Transgenic erecta-105 plants expressing ERECTA::ERL1 or ERECTA::ERL2 displayed phenotypes, such as elongated inflorescence and pedicels, nearly identical to the wild-type plants. Therefore, both ERL1 and ERL2 can substitute for ERECTA function when expressed in the tissue- and cell types that normally express ERECTA, suggesting that two ERLs are capable of perceiving and transducing the same signal as ERECTA.

ERECTA, ERL1, and ERL2 display overlapping, but unique expression patterns: Inability of ERL1 and ERL2 to complement erecta mutants while expressed under their endogenous promoter suggests differences in expression patterns. At the same time, if ERL1 and ERL2 are the RLKs whose function is inhibited by the dominant-negative ERECTA fragment expressed under the control of the ERECTA promoter, they would be expected to be expressed, at least in part, in an overlapping manner with ERECTA. To clarify these points, developmental expression of ERL1 and ERL2 was analyzed.

RT-PCR analysis showed that, similar to ERECTA, expression levels of two ERLs were higher in developing organs, including bud clusters, flowers, siliques, and young rosettes, lower in mature aboveground organs, such as leaves, stems, and pedicels, and barely detectable in roots. However, expression levels of ERL1 and ERL2 in mature organs were much lower than ERECTA.

To examine the organ- and tissue-specific expression patterns of ERL1 and ERL2 in detail, promoter fragments of ERL1 (4.1 kb) and ERL2 (4.4 kb) were fused transcriptionally to the GUS gene and introduced in Arabidopsis wild-type plants. Expression pattern of ERECTA::GUS, ERL1::GUS, and ERL2::GUS marks the actively-proliferating organs. At the vegetative stage, both ERL1::GUS and ERL2::GUS were strongly expressed in the shoot meristem, leaf primordia and juvenile leaves. At the reproductive stage, GUS expression was detected in the young developing flowers up to stage 12 for ERECTA and ERL2 and up to stage 14 for ERL1. ERECTA::GUS and ERL1::GUS were detected in inflorescence meristem and visibly up-regulated during flower initiation and formation of flower organs. The GUS expression was also detected in cells that will differentiate into pedicels. In developing flowers, the expression of ERECTA, ERL1, and ERL2 was in the actively growing region of the floral organs and thus altered dynamically as the developmental stages of the floral organs progressed. At the early stages, all three genes were expressed in an overlapping manner in all flower organs. Later, their expression became confined to different subsets of proliferating tissues. For instance, at flower stage 11, ERECTA::GUS was largely expressed in the mesocarp and to a lesser degree in ovules, while ERL1::GUS was expressed predominantly in ovules and ERL2::GUS in style and ovules. The finding that ERL1 and ERL2 display overlapping but unique expression patterns suggests their roles as parallel or a subset of the ERECTA signaling pathway.

Isolation of the null alleles of ERL1 and ERL2: To investigate the roles of the two ERLs in Arabidopsis growth and development, T-DNA-tagged, loss-of-function alleles of ERL1 and ERL2 were identified. erl1-2 has a T-DNA insertion at nt +3410 from the translation initiation codon within exon 18, which encodes the 16th LRR. erl2-1 has a T-DNA insertion at nt +2454 from the translation initiation codon within exon 14, which encodes the 12th LRR. The T-DNA insertions in erl1-2 and erl2-1 are associated with a deletion of 59 and 76 nucleotides, respectively, suggesting that they represent the knockout (null) alleles. Indeed, no detectable ERL1 transcripts were observed in erl1-2 and no ERL2 transcripts were observed in erl2-1 by RT-PCR.

The absence of either ERL1 or ERL2 transcripts had no effect on ERECTA expression levels. Similarly, expression levels of ERL1 and ERL2 were not altered in erecta-105 plants, which do not express any ERECTA transcripts. The lack of up- or down-regulation among three ERECTA-family LRR-RLKs implies that their signaling pathways do not constitute an interconnected feedback loop.

Both ERL1 and ERL2 are redundant: erl1-2 and erl2-1 were further subjected to phenotypic characterization. A morphogenetic analysis was performed on fully-grown eight-week-old plants. erl1-2 and erl2-1 single mutant plants were indistinguishable from wild-type plants (Table 3). Their inflorescence undergoes elongation of the internodes between individual flowers and they all displayed normal length of petioles, stems, pedicels, and siliques (Table 3). The lack of any visible phenotype suggests that ERL1 and ERL2 are redundant.

TABLE 3
Morphogenetic Analysis of er1-2, er2-1, and er-105 Plants

| | Plant Height | Pedicle Length | Silique Length |
|---|---|---|---|
| Genotype | (cm +/− SD) | (mm +/− SD) | (mm +/− SD) |
| wild type | 37.6 +/− 3.0 | 8.2 +/− 0.9 | 15.87 +/− 0.7 |
| erl1-2 | 37.1 +/− 1.9 | 8.8 +/− 0.7 | 16.2 +/− 0.8 |
| erl2-1 | 37.9 +/− 3.2 | 8.1 +/− 0.7 | 16.0 +/− 0.5 |
| erl1-2 erl2-1 | 36.9 +/− 4.5 | 9.0 +/− 0.7 | 14.6 +/− 0.7 |
| er-105 | 22.7 +/− 1.9 | 3.9 +/− 0.4 | 10.8 +/− 0.5 |
| er-105 er1-2 | 24.2 +/− 2.2 | 3.3 +/− 0.4 | 6.9 +/− 0.9 |
| er-105 er2-1 | 16.2 +/− 1.1 | 3.7 +/− 0.4 | 8.9 +/− 0.5 |
| er-105 er1-2 er2-1 | 1.6 +/− 1.5 | N/A | N/A |

Since ERL1 and ERL2 appear to have undergone recent gene duplication, it may be necessary to remove both gene products in order to reveal their biological functions. To test this hypothesis, an erl1-2 erl2-1 double mutant was generated. erl1-2 erl2-1 plants did not exhibit any visible phenotype (Table 3). While ERL1 and ERL2 are capable of rescuing the growth defects of erecta-105, erl1 and erl2 single mutants, as well as the erl1 erl2 double mutant failed to confer any developmental phenotype. The finding suggests that loss-of-function of ERL1 and ERL2 is masked by the presence of the functional ERECTA gene.

Duplications of developmental regulatory genes followed by subsequent mutation and selection are thought to have driven the morphological diversity in multicellular organisms. Acquisition of novel gene functions occurs by alteration of protein function or of gene expression patterns. The fact that ERL1 and ERL2 are capable of substituting for ERECTA activity when driven by the ERECTA promoter and terminator indicates that specificity among ERECTA, ERL1, and ERL2 largely lie in their cis-regulatory elements rather than protein-coding regions. The dominance of cis-regulatory sequences over protein-coding regions in functional specification among closely-related multigene families has been documented for transcription factors regulating development, such as: Hox genes in mouse development, Myb-genes WER and GL1 in Arabidopsis epidermal patterning, and AGAMOUS-family MADS-box genes in Arabidopsis ovule development (Greer et al. (2000) Nature 403:661-5; Lee & Schiefelbein (2001) Development 128:1539-46; Pinyopich et al. (2003) Nature 424:85-8).

Because ERECTA-family genes encode putative receptor kinases, their functional equivalence indicates that ERECTA, ERL 1, and ERL2 are capable of perceiving the same ligand(s) and eliciting the same downstream response(s). This raises a novel view on how the extent of organ growth is monitored by cell-cell signaling in Arabidopsis. The prevalent model based upon Drosophila wing development is that final organ size is determined by the steepness of morphogen gradients (Day & Lawrence (2000) Development 127:2977-87). According to this model, concentration gradients of ligands, such as Dpp or Wg, dictate where and when cells proliferate. In contrast, it is hypothesized that tissue-specific and redundant expression of functionally equivalent receptors plays a regulatory-role in coordinating Arabidopsis aerial organ growth. In the organ primordium, where cells are proliferating ubiquitously, uniform expression of all three ERECTA-family LRR-RLKs maximizes the organ growth. As the organ matures, localized and non-redundant expression of each RLK fine-tunes local, subtle growth for elaboration of final form and size. Transient, non-overlapping expression of ERECTA, ERL1, and ERL2 in a developing gynoecium reflects such intricate local growth patterns, since growth and differentiation of distinct tissues, such as stigma, style valves, and ovules, must occur concomitantly during carpel development (Ferrandiz et al. (1999) Ann. Rev. Biochem. 68:321-54). This view is in accordance with previous findings that strength of the ERECTA pathway specifies final organ size in a quantitative manner (Lease et al. (2001) New Phytol. 151:133-44; Torii et al. (1996) Plant Cell 8:735-46; Torii et al. (2003) in Morphogenesis and Patterning of Biological Systems (ed. T. Sekimura, Tokyo, Japan: Springer-Verlag) pp. 153-64).

A recent molecular evolutionary study implies that the RLK superfamily underwent radical expansion within the plant lineage. The existence of more than 600 RLK-coding genes in the Arabidopsis genome is in sharp contrast with the small numbers of their counterparts (Pelle/IRAK family) in animals, 3 in mice and 4 in humans (Shiu & Bleecker (2003) Plant Physiol. 132:530-43). Consistently, gene duplication events among RLK sub-families have been documented (Baudino et al. (2001) Planta 213:1-10; Nishimura et al. (2002) Nature :426-9; Searle et al. (2003) Science 299:109-12; Shiu & Bleecker (2003) Plant Physiol. 132:530-43; Yamamoto & Knap (2001) Mol. Biol. E vol. 18:1522-31; Yin et al. (2002) Proc. Natl. Acad. Sci. U.S.A. 99:9090-2), but their biological significance is not fully understood. These findings confirm the effectiveness of the dominant-negative approach, and further provides framework for understanding functional redundancy among recently duplicated plant RLK gene families.

erl1 and erl2 enhance a subset of erecta defects in a unique manner: To uncover the developmental role of ERL1 and ERL2 in the absence of functional ERECTA, erl1 and erl2 mutations were introduced into erecta-105 plants (Torii et al. (1996) Plant Cell 8:735-46; Torii et al. (2003) in Morphogenesis and Patterning of Biological Systems (ed. T. Sekimura, Tokyo, Japan: Springer-Verlag) pp. 153-64). Both erl1 and erl2 enhanced the erecta defects in a unique manner. The erl1-2 mutation notably exaggerated the silique and pedicel elongation defects of erecta-105. erecta-105 erl1-2 double mutant plants developed very short, blunt siliques and short pedicels (Table 3), both of which are reminiscent of a subset of the phenotype conferred by the dominant-negative delta-Kinase (Shpak et al. (2003) Plant Cell 15:1095-1110). The presence of the erl1-2 mutation did not significantly affect the height of erecta-105 plants (Table 3).

By contrast, the erl2-1 mutation primarily enhanced the internodal elongation defects of erecta. erecta-105 erl2-1 double mutant plants were much shorter than erecta-105 and developed very compact inflorescence with tightly clustered flowers and flower buds at the tip (Table 3). The architecture of erecta-105 erl2-1 inflorescence resembles that of the transgenic erecta-105 expressing delta-Kinase (Shpak et al. (2003) Plant Cell 15:1095-1110). In addition, the erecta-105 erl2-1 siliques were slightly shorter than those of erecta-105 (Table 3).

The morphology of the silique tip was analyzed in detail. The erecta-105 silique tip has a blunt appearance due to a wide style that protrudes less from the valves than wild type. Both erl1 and erl2 mutations exaggerated this characteristic erecta silique phenotype, with even wider valves and shorter, broader styles. This indicates that the enhancement of the silique phenotype by erl1-2 and erl2-1 are not due to general elongation defects unrelated to the ERECTA pathway. These results suggest that ERL1 and ERL2 act in an overlapping but distinct subset of the ERECTA signaling pathway in regulating inflorescence architecture and organ shape. The specific sites of enhancement of the erecta phenotype by either erl1 or erl2 mutation appear to correspond to the expression domains of these two LRR-RLKs, which are weaker and confined to a subset of ERECTA expression domains.

Synergistic interaction of ERECTA, ERL1, and ERL2 in promoting organ growth and flower development: To understand the biological function of the ERECTA-family LRR-RLK as a whole, an erecta-105 erl1-2 erl2-1 triple mutant was generated. For this purpose, F2 plants that were homozygous for erecta and erl2 but heterozygous for erl1 were self-fertilized. A subsequent F3 population segregated extremely dwarf, sterile plants at ˜25% ratio (dwarf plants/total=74/315, χ²=0.382, p=0.537), suggesting that they may be the triple mutant. To test this hypothesis, genotypes of 86 F3 plants were analyzed. Among 63 compact, fertile plants, 40 were heterozygous for erl1, 23 were wild type for ERL1, and none were homozygous for erl1, consistent with the expected 2:1 ratio (χ²=0.286, p=0.593). Oby contrast, all 23 extremely dwarf, sterile plants were homozygous for erl1 and thus carried erecta-105 erl1-2 erl2-1 triple mutations. Furthermore, progeny of the F3 siblings with a genotype erecta-105 ERL1 erl2-1 failed to segregate extremely dwarf plants (0/227 scored). These results provide statistical evidence that the triple mutations confer severe growth defects (Fisher's exact test, p<0.00000001).

The phenotype of erecta-105 erl1-2 erl2-1 triple mutant plants during postembryonic development was analyzed. The striking effects of erecta-105 erl1-2 erl2-1 mutations on organ growth can be seen in all aboveground organs and are evident soon after germination, at a time when cells start to divide. Decreased cotyledon growth is notable in 4-day-old erecta-105 erl1-2 erl2-1 seedlings and it is more striking in 12-day-old seedlings, which have small, misshaped cotyledons with very short petioles. Growth of primary leaves is strongly diminished in the triple mutant seedling, while leaf primordia are forming on a flank of the SAM. Interestingly, the triple mutations do not affect hypocotyl elongation, which occurs solely due to cell elongation (Gendreau et al. (1997) Plant Physiol. 114:295-305). At a later stage of vegetative development, erecta-105 erl1-2 erl2-1 plants form a small rosette with small, round leaves that lack petiole elongation. Transition to flowering occurs approximately at the same time in wild-type, erecta-105, and erecta-105 erl1-2 erl2-1 plants, suggesting that mutations in three ERECTA-family genes do not affect phase transition.

The phenotypes of triple mutant plants at the reproductive stage are variable. While the main inflorescence stem always exhibits severe elongation defects, axillary branches occasionally show various degrees of phenotypic rescues. A variable level of phenotypic rescue was also noticeable in flowers and pedicels at a later stage of axillary inflorescence development. Flowers with stronger phenotypes have reduced number of organs with occasional fusion of organs, and their pedicels are either absent or too short to be detected. Those with weaker phenotype have all four organs formed but they are smaller in size and incompletely developed. Such flowers have extremely short, but recognizable pedicels. Unlike erecta-105, the triple mutant flowers develop cylindrical, needle-like petals that lack polar expansion, very short gynoecium, and small anthers that are incompletely differentiated, all of which are visible at stage 9 flowers as well as at mature flowers. Ovule development is either absent or aborted at a very early stage, and this is consistent with the overlapping expression of ERECTA, ERL1 and ERL2 in developing ovules. These phenotypes are much more severe than erecta-105 plants expressing delta-Kinase, suggesting that the dominant-negative interference previously shown (Shpak et al. (2003) Plant Cell 15:1095-1110) was not complete. The results demonstrate that ERECTA, ERL1 and ERL2 genes interact synergistically and that these three ERECTA-family LRR-RLKs as a whole specify the proper growth and differentiation of all aboveground organs.

erecta-105 erl1-2 erl2-1 triple mutants are defective in cell proliferation: To unravel the cellular basis of reduced organ growth, cellular morphology in petals and pedicels was examined. The Arabidopsis petals have a simple cell layer structure with epidermal cells that are uniform in size and shape (Bowman (1993) Arabidopsis: An Atlas of Morphology and Development (Springer-Verlag, New York)). While petals of erecta-105 erl1-2 erl2-1 plants are very small and filamentous in shape, their abaxial epidermis cells are slightly larger than in erecta-105 petals.

As reported previously, erecta-105 pedicels have a reduced number of expanded cortex cells (Shpak et al. (2003) Plant Cell 15:1095-1110). Similar to erecta-105, erecta-105 erl1 and erecta-105 erl2 double mutations and erecta-105 erl1-2 erl2-1 triple mutations confer reduced cell numbers associated with enlarged and irregular cell shape in the cortex. Interestingly, erecta-105 erl1-2 and erecta-105 erl1-2 erl2-1 mutations led to disorganized cell growth in the cortex. Cells are irregular in size and shape and leave gaps in between. This phenotype is similar to transgenic erecta-105 plants expressing delta-Kinase (Shpak et al. (2003) Plant Cell 15:1095-1110). Cell numbers in a longitudinal cortex file are severely reduced in the mutants, with a concomitant decrease in the final pedicel length (Table 4). erecta-105 pedicel has 3 times fewer cells per longitudinal row and erecta-105 erl1-2 erl2-1 has 11 times less compared to the wild type (Table 4). These results demonstrate that organ growth defects of erecta erl1 erl2 are largely due to a decrease in cell number and suggest that ERECTA-family genes promote cell proliferation during organ growth.

TABLE 4
Number of Cells in the Longitudinal Cortex
File of Mature Pedicels of Plants

| Genotype | Number of Cells |
|---|---|
| wild type | 487 |
| er-105 | 169 |
| er-105 er1-2 | 123 |
| er-105 er2-1 | 140 |
| er-105 er1-2 er2-1 | 45 |

Molecular analysis of erecta erl1 erl2 inflorescence suggests a novel mechanism for organ growth regulation: To understand the molecular basis of organ growth/cell number defects conferred by the triple mutations, the expression levels of four transcription factor genes that regulate shoot- and floral organ size were analyzed. ANT acts to prolong duration of cell proliferation during lateral organ development, and its loss-of-function confers reduced organ size (Elliott et al. (1996) Plant Cell 8:155-68; Mizukami & Fischer (2000) Proc. Natl. Acad. Sci. U.S.A. 97:942-7). Loss-of-function mutations in SHOOTMERISTEMLESS (STM) and WUSCHEL (WUS) homeobox genes cause a decrease in the number of meristem cells and growth defects of lateral organs (Laux et al. (1996) Development 122:87-96; Long et al. (1996) Nature 379:66-9). The BREVIPEDICELLUS (BP) locus encoded by the KNAT1 homeobox gene interacts synergistically with ERECTA in promoting internodal elongation and floral organ size (Douglas et al. (2002) Plant Cell 14:547-58). Semi-quantitative RT-PCR analysis of flower and bud clusters reveals that erecta-105 erl1-2 erl2-1 triple mutations do not affect expression levels of ANT, STM, or KNAT1. WUS expression was slightly reduced in the triple mutant background. However, such a slight reduction does not likely account for severe defects in shoot- and floral organ growth and internodal elongation in the triple mutants.

It is known that ANT leads to prolonged expression of D-type cyclins, which control the entry to cell cycle progression at the G1 stage (Cockcroft et al. (2000) Nature 405:575-9; Dewitte & Murray (2003) Annu. Rev. Plant Biol. 54:235-64; Mizukami & Fischer (2000) Proc. Natl. Acad. Sci. U.S.A. 97:942-7. Transcript levels of two D-type cyclins, CycD2;1 and CycD3;1, were not significantly altered by the triple mutations. This is consistent with the notion that the control of organ size by ERECTA-family RLKs involves mechanism other than the pathway mediated by ANT. Taken together, the results suggest that three ERECTA-family LRR-RLKs promote cell proliferation via a novel mechanism.

The most prominent feature of erecta single and erecta erl double- and triple mutations is a reduction in aerial organ size due to reduced cell numbers. In theory, cell numbers in lateral organs can be regulated by affecting number of SAM cells available for recruitment to organ primordia, by promotion of cell proliferation, or by prolonging duration (window) of cell proliferation during organ growth. These results suggest that ERECTA-family genes most likely function in promotion of cell proliferation. The triple mutations do not likely disturb SAM function: Even a strikingly tiny leaf of the triple mutant initiates and increases in size with the same timing as wild type. Consistently, WUS and STM expression levels are not significantly altered by the mutation. Furthermore, expression of cyclin D2, whose overexpression confers increase in growth rate by accelerating primordia initiation in the SAM (Cockcroft et al. (2000) Nature 405:575-9), is not affected in the triple mutant background. It is also unlikely that ERECTA-family genes prolong duration of cell proliferation, as erecta erl1 erl2 mutations do not lead to early secession of organ growth. Consistently, expression of ANT, which promotes the meristematic competency of developing organs through prolonged expression of cyclin D (Mizukami & Fischer (2000) Proc. Natl. Acad. Sci. U.S.A. 97:942-7), is not down-regulated by the triple mutations.

In addition to growth defects, erecta erl1 erl2 plants exhibit aberrant floral organ differentiation, notably in anthers and ovules. This may be due to inhibited primordia growth, which results in a diminished supply of progenitor cells for tissues that differentiate at later stages of flower development. Alternatively, ERECTA-family genes as a whole may play some specific roles in flower organ differentiation. In this regard, it is interesting that ANT, which also specifies organ size but via distinct mechanism, is known to be required for proper ovule differentiation and floral organ identity (Elliott et al. (1996) Plant Cell 8:155-68; Klucher et al. (1996) Plant Cell 8: 137-53; Krizek et al. (2000) Plant Cell 12:1357-66).

In contrast to the main inflorescence, axillary branches of erecta erl1 erl2 plants displayed various degrees of phenotypic rescue (FIG. 6C). One possibility that explains such rescue could be the indirect effects caused by premature termination of the SAM (the main inflorescence) that relieves the growth of axillary branches via ERECTA-independent mechanisms (Leyser, 2003). Alternatively, control of axillary branch development may involve factors that possess partially redundant function with ERECTA-family receptor-like kinases. Such factors might be more distantly related receptor-like kinases and/or gene products with no primary sequence similarity to ERECTA. It is noteworthy that ERECTA, ERL1, and ERL2 belong to the LRR-XIII family with four additional, distantly-related members (Shiu & Bleecker (2001) Proc. Natl. Acad. Sci. U.S.A. 98:10763-8).

The increase in cell size in erecta single- and erecta erl double- and triple mutants is likely to be secondary to reduction in cell number. When cell proliferation is decreased, the total mass checkpoint often leads to decreased inhibition of cell growth, resulting in increased cell size (Conlon & Raff (1999) Cell 96:235-44; Day & Lawrence (2000) Development 127:2977-87; Mizukami (2001) Curr. Opin. Plant Biol. 4:533-9; Nijout (2003) Dev. Biol. 261:1-9; Potter & Xu (2001) Curr. Opin. Genet. Dev. 11:279-86). The expression of ERECTA, ERL1 and ERL2 in actively dividing tissues holds up well with their proposed function in cell proliferations. Interestingly, a striking decrease in cortex cell numbers occurs only at the vertical cell files, while compensatory cell expansion is much more notable along the radial axis. As a consequence, erecta single and erecta erl double mutants develop organs with a characteristic shapes that are shorter but thicker. Therefore, ERECTA-family RLKs may respond to elusive signals that determine the longitudinal dimension of organ growth. Alternatively, it is possible that ERECTA-family RLKs may possess specific roles in regulation of cell shape and polarity in addition to cell division.

Remarkably, the cortex cells in erecta-105 erl1-2 and erecta-105 erl1-2 erl2-1 pedicels are disorganized with erratic shape and uneven size. The cellular phenotype suggests that ERECTA-family RLKs play a fundamental role in coordinating cell proliferation within tissues. In this respect, ERECTA-family RLKs are distinct from a receptor for a peptide-hormone phytosulfokine (PSK), which also encodes an LRR-RLK (Matsubayashi et al. (2002) Science 296:1470-2). While the PSK-receptor stimulates rapid, unorganized cell proliferation in culture cells, ERECTA-family RLKs mediate cell proliferation in the context of whole organism. Consistent with this hypothesis, ERECTA-family genes are not highly expressed in Arabidopsis culture cells.

### EXAMPLE 3

This Example describes an exemplary method of the invention for modulating the growth or form of a plant by expressing a truncated form of the receptor kinase ERECTA in transgenic plants under the control of a 35S promotor.

Methods

CaMV35S delta-kinase constructs: PCR was performed using pKUT195 (a plasmid harboring a full-length ERECTA genomic fragment) using the following primer pair: ERcode5 (5′ CGG AAT TCA CTA GTA CCA TGG CTC TGT TTA GAG ATA TTG 3′, SEQ ID NO:82) and ERg3476rc (5′ ATA CAA AAC CTG GAA GGC AGT G 3′, SEQ ID NO:83). The amplified fragment was inserted in NcoI/SpeI-digested pKUT195, and the new plasmid was named pKUT195.NcoI. The sequence was confirmed. pKUT195.NcoI was subsequently digested with ClaI, treated with T4 polimerase to blunt the end, and further digested by NcoI. The SmaI/NcoI cleaved 35S-promoter fragment from pKUT413 was inserted into pKUT195.NcoI. The resulted plasmid was named pESH104. pESH104 was digested with SalI and fragment was inserted in SalI digested pZP222 to generate a construct that allows expression of a full-length ERECTA driven by the CaMV 35S promoter in the T-DNA transformation vector. The plasmid was named pESH232.

To generate CaMV35S ERECTAdelta-kinase-GFP construct, pESH204 was digested with EcoRI and then inserted into EcoRI-digested pESH232. The orientation of insert was confirmed.

To generate CaMV35S ERECTAdelta-kinase, pESH104 was partially digested with XbaI, and XbaI fragment derived from pESH201 (ERECTAdeltaKinase driven by the endogenous ERECTA promoter) was inserted. The orientation was confirmed and the plasmid was named pKUT584. To generate CaMV35S ERECTAdelta-kinase that is eptope-tagged with 3xcMyc, pESH104 was partially digested with XbaI, and XbaI fragment derived from pESH215 (ERECTAdeltaKinase-3xcMyc driven by the endogenous ERECTA promoter) was inserted. The orientation was confirmed and the plasmid was named pKUT585.

Results

The phenotype was essentially identical to that obtained using pESH201 (ERECTA::ERECTAdeltaKinase), as described in EXAMPLE 1. The phenotype was somewhat weaker. The plants were compact but fertility was not affected.

### EXAMPLE 4

This Example describes an exemplary method of the invention for modulating the growth or form of a plant by expressing a truncated form of the receptor kinase ERECTA in transgenic tobacco plants.

Methods

Generation of ACTINpromoter-ERECTAdeltaKinase construct: To drive expression of ERECTAdeltaKinase under the control of rice Actin1 promoter, both pKUT584 and pKUT585 were digested with EcoRI and BamH1, blunt ended, and ligated into SmaI-digested pact-nos/Hm2 (Zhang et al. (1991) Plant Cell 3:1155-65). The resulted plasmids were named pKUT586 (ACTINprom-ERECTAdeltaKinase-nosTerm) and pKUT587 (ACTINprom-ERECTAdeltaKinase-3xcMyc-nosTerm).

Generation of ERECTApromoter-ERL1deltaKinase construct: To drive expression of ERL1deltaKinase under the control of ERECTA promoter and terminator, the PCR was performed on pKUT600 (ERL1deltaKinase without any stop codon in pBluescriptIISK+ vector, Torii, unpublished) using primers ERL1_{—}433XbPc (5′ gctctagacATGttGGAGAAGATGCAGCGAATGGTT 3′, SEQ ID NO:84) and ERL1_{—}4828StopXbRC (5′ gctctagactaGGAGCCTTGTAGAATCTTCTTCT 3′, SEQ ID NO:85). The PCRed fragment was digested with XbaI and inserted into XbaI-digested pBluescriptIISK+ vector. The sequence was confirmed. The XbaI-digested ERL1deltaKinase fragment was inserted into SpeI-digested pKUT522 (ERECTA promoter-terminator cassette in plant T-DNA transformation vector). The orientation of the fragment was confirmed.

Generation of Transgenic Tobacco Plants Expressing delta-Kinase: All steps in the procedure were performed using sterile conditions.

1. Growth of sterile tobacco seedlings: Tobacco seeds (Nicotiana tabacum) were sterilized for 15 minutes in the solution of 33% household bleach and 0.1% Triton X-100, washed 3 times in the sterilized water and planted on Magenta boxes with NT-1 medium (1×MS salts (Sigma), 30 g/L sucrose, 1 mg/L thiamine-HCl, 100 mg/L Myo-Inositol, 180 mg/L KH2PO4, pH5.2) containing 0.75% of Bactoagar.

2. Agrobacterium infection: After 3 to 4 weeks several leafs of tobacco plants were removed to a Petri dish containing 50 ml of NT-1 medium, wounded with a paper punch and co-cultivated in the dark at 250 C with appropriate strain of Agrobacteria (1 ml of overnight culture grown in LB medium with appropriate antibiotics). After 2 days of co-cultivation leaves were washed 2 times in NT-1 medium and transferred to Magenta boxes with NT-1 medium containing 0.75% of Bactoagar, 0.6 mg/L 6-benzylaminopurine, 40 mg/L timentin and 36 mg/L gentamycin.

3. Excision and rooting of gentamycin resistant shoots: After 4-6 weeks appeared shoots were excised from leaf discs and transferred to Magenta box with NT-1 medium containing 0.75% of Bactoagar, 40 mg/L timentin and 36 mg/L gentamycin. The shoots which were able to form roots during following 3 weeks were transferred to the soil.

The presence of the delta-kinase transgene in ten T2 transgenic plants from each line was examined by PCR analysis.

Results

The immediate T1 generation of transgenic tobacco plants transformed with delta-kinase ERECTA exhibited striking dwarf phenotypes. Six T1 transgenic tobacco plants were analyzed, two with severe phenotypes (L5 and L9), two with weak phenotypes (L43 and L44), and two with no apparent phenotype (L3 and L10). Each line was self-fertilized, and the inheritance of the phenotype was analyzed at the subsequent, T2 generation.

All T2 progeny of L5 and L9 (31 and 12 plants, respectively) inherited dwarfism, as shown in Table 5. All ten plants per line that were subjected to genomic PCR analysis contained the Arabidopsis delta-kinase ERECTA transgene.

TABLE 5
Morphogenetic Analysis of delta-Kinase
ERECTA Transgenic Tobacco Plants

| Tobacco Line | Plant Height (cm +/− SD) |
|---|---|
| L5 | 20.5 +/− 9.4 |
| L9 | 22.8 +/− 9.2 |
| L43 | 75.1 +/− 13.1 |
| L44 | 83.9 +/− 18.4 |
| L3 | 101.6 +/− 11.9 |
| L10 | 90.8 +/− 6.9 |
| control | 99.5 +/− 6.4 |

A majority of the T2 progeny of L43 displayed a weak phenotype: the plants exhibited compact stature with highly clustered flower buds, as shown in Table 5. All ten plants that were subjected to genomic PCR analysis contained the Arabidopsis delta-kinase ERECTA transgene. T2 progeny of L44 segregated plants with a weak phenotype (6 plants, Table 5) and with no phenotype (4 plants). The weak phenotype was linked with the presence of the delta-kinase ERECTA transgene.

A majority of the T2 progeny of L3 retained the delta-kinase ERECTA transgene but did not display a growth phenotype (Table 5). T2 progeny of L10 lost the delta-kinase ERECTA transgene.

These results show that alteration of tobacco growth morphology by Arabidopsis delta-kinase ERECTA was stably inherited. All plants with strong phenotypes retained the delta-kinase ERECTA transgene. Not all plants that had the transgene exhibited a growth phenotype. This may be due to differences in the expression of the delta-kinase ERECTA transgene.

While the preferred embodiment of the invention has been illustrated and described, it will be appreciated that various changes can be made therein without departing from the spirit and scope of the invention.

1. A method for modulating plant height and organ shape, comprising the step of expressing a transgene in a plant, wherein the transgene encodes an ERECTA-like protein lacking an active kinase domain and wherein expression of the transgene modulates plant height or organ shape.

2. The method of claim 1, wherein the ERECTA-like protein is an ERECTA protein.

3. The method of claim 2, wherein the ERECTA protein comprises the sequence provided in SEQ ID NO:2.

4. The method of claim 1, wherein the ERECTA-like protein is an ERL1 protein.

5. The method of claim 4, wherein the ERL1 protein comprises the sequence provided in SEQ ID NO:6.

6. The method of claim 1, wherein the ERECTA-like protein is an ERL2 protein.

7. The method of claim 6, wherein the ERL2 protein comprises the sequence provided in SEQ ID NO:8.

8. The method of claim 1, wherein the ERECTA-like protein lacking an active kinase domain comprises the sequence provided in SEQ ID NO:4.

9. The method of claim 1, wherein the ERECTA-like protein lacking an active kinase domain comprises the sequence provided in SEQ ID NO:10.

10. The method of claim 1, wherein the ERECTA-like protein lacking an active kinase domain comprises the sequence provided in SEQ ID NO:12.

11. The method of claim 1, wherein the ERECTA-like protein lacking an active kinase domain comprises the sequence provided in SEQ ID NO:86.

12. The method of claim 1, wherein the ERECTA-like protein lacking an active kinase domain comprises the sequence provided in SEQ ID NO:87.

13. The method of claim 1, wherein the ERECTA-like protein lacking an active kinase domain comprises the sequence provided in SEQ ID NO:88.

14. The method of claim 1, wherein the transgene is expressed in the shoot apical meristem.

15. The method of claim 1, wherein the plant is a crop plant.

16. The method of claim 1, wherein expressing the transgene produces a dwarf plant.

17. A method for enhancing the yield of a crop plant, comprising the steps of:
(a) introducing a transgene into a crop plant, wherein the transgene encodes an ERECTA-like protein lacking an active kinase domain and wherein expression of the transgene enhances the yield of the crop plant; and
(b) growing the transgenic crop plant under conditions in which the transgene is expressed to enhance the yield of the crop plant.

18. The method of claim 17, wherein the crop plant is a rice plant or a canola plant.
